(19) United States
(12) Patent Application Publication
(10) Pub. No.: US 2025/0121061 A1
(43) Pub. Date: Apr. 17, 2025

(54) CHIMERIC ANTIGEN RECEPTOR INCLUDING CD30-DERIVED INTRACELLULAR SIGNALING DOMAIN, IMMUNE CELL EXPRESSING SAME, AND USE THEREOF

(71) Applicant: VIGENCELL INC., Seocho-gu Seoul (KR)

(72) Inventors: Hyun-Il CHO, Suwon-si Gyeonggi-do (KR); Jung-Min HA, Songpa-gu Seoul (KR); Sang Eun LEE, Dobong-gu Seoul (KR); Chung Hyo KANG, Manan-gu, Anyang-si Gyeonggi-do (KR); In Sil SONG, Jangan-gu, Suwon-si Gyeonggi-do (KR)

(21) Appl. No.: 18/834,896

(22) PCT Filed: Mar. 30, 2023
(86) PCT No.: PCT/KR2023/004241

§ 371 Date: Jul. 31, 2024

(30) Foreign Application Priority Data
Mar. 30, 2022 (KR) 10-2022-0039648

(51) Int. Cl. A61K 40/11; A61K 40/31; A61K 40/42; A61P 35/02; C07K 14/705; C07K 14/725; C07K 16/28; C07K 16/30; C12N 5/0783

(52) U.S. Cl. CPC A61K 40/11; A61K 40/31; A61K 40/4202; A61K 40/4211; A61K 40/4254; A61K 40/4255; A61K 40/4261; A61P 35/02; C07K 14/7051; C07K 14/70578; C07K 16/2803; C07K 16/2878; C07K 16/30; C07K 16/303; C12N 5/0636; C07K 2317/73; C07K 2319/02; C07K 2319/03; C12N 2510/00

(57) ABSTRACT

The present invention relates to a chimeric antigen receptor including a CD30-derived intracellular signaling domain, immune cells expressing same, and uses thereof. More specifically, the present invention is designed to use a chimeric antigen receptor including a portion of the sequence of TRAF-binding domain within the CD30 domain as an intracellular signaling domain to increase the proliferation and survival of immune effector cells, thereby providing an effect of enhancing antitumor efficacy and cytokine secretion.

### SEQUENCE LISTING

The text of the computer readable sequence listing filed herewith, titled “X23E10C0015_sequence listing”, created Mar. 30, 2023, having a file size of 263,560 bytes, is hereby incorporated by reference in its entirety.

### BACKGROUND

### 1. Field of the Invention

The present invention relates to a chimeric antigen receptor including a CD30-derived intracellular signaling domain, an immune cell expressing the same, and use thereof.

### 2. Discussion of Related Art

Over the past few decades, surgery, radiation therapy, chemotherapy, and the like have been developed as methods to treat cancer, but limitations such as serious adverse effects and resistance due to mutations have been revealed. Recently, immune checkpoint inhibitors, adaptive immune cell therapy, and the like have been attracting attention as promising cancer treatment methods.

Chimeric antigen receptor (CAR)-T cell therapy is a treatment method in which cytotoxic T cells are removed from the body, genetically modified to express a CAR that can recognize a specific antigen, and reintroduced into the body. Through this, it is possible to recognize antigens expressed by cancer cells, but not limited to the major histocompatibility complex (MHC), and selectively kill them. A CAR consists of a single chain variable fragment (scFv) that can recognize a specific antigen, a spacer domain, a transmembrane domain, and an intracellular signaling domain that transmits T cell activation signals.

The first-generation CAR consists of a scFv that recognizes an antigen and a single intracellular signaling domain, and as the signaling domain, CD3 zeta, which is the main signaling chain of the T cell receptor (TCR), or FcR-gamma, which is the signaling chain of the activated Fc receptor, was used. However, the first-generation CAR-T cells lacked clinical efficacy due to limited proliferation and survival ability in the body. To overcome this, a second-generation CAR, formed by adding a signaling region of costimulatory receptors such as CD28, 4-1BB, ICOS, OX40, and CD27 to CD3 zeta, and a third-generation CAR, formed by combining two different types of costimulatory domains, were developed. The second-generation and third-generation CARs exhibited improved anticancer effects through active proliferation and long-term survival in vivo. In addition, in order to further improve the effect on solid tumors, research is continuously underway on a fourth generation CAR, which is based on the second or third-generation CAR and co-expresses cytokines or costimulatory ligands that help the activation of T cells, or a fifth generation CAR, which includes a technology to inhibit human leukocyte antigen (HLA) or TCR genes.

To improve the efficacy of CAR-T cells, research is being conducted to optimize the domains that make up CARs. In particular, it is known that the costimulatory domain plays an important role in CAR-T cell proliferation, expansion, maintenance, antitumor activity, cytokine secretion, and the like. Commonly used costimulatory domains include the immunoglobulin (Ig) superfamily, such as CD28 and ICOS (CD278), and the tumor necrosis factor receptor (TNFR) superfamily, including 4-1BB (CD137), OX40 (CD134), and CD27. The Ig superfamily costimulatory domains activate phosphatidylinositol 3-kinase (PI3K), which then activates protein kinase B (Akt) and nuclear factor κB (NF-κB) signaling pathways. On the other hand, the TNFR superfamily costimulatory domains activate the NF-κB signaling pathway through various types of TNF receptor associated factors (TRAFs).

As a conventional CAR-T cell therapy, CAR-T cell therapy targeting human CD19 exhibited impressive therapeutic effects against blood cancers such as acute lymphoblastic leukemia (ALL), diffuse large B-cell lymphoma (DLBCL), and non-Hodgkin's lymphoma, but is less effective against solid cancers. This is because CAR-T cells lack persistence in the body and have difficulty reaching solid tumors (trafficking), and solid tumors are surrounded by an immunosuppressive tumor microenvironment.

Therefore, a strategy that can increase the in vivo proliferation and survival of CAR-T cells is needed for excellent therapeutic effects even on solid tumors.

### SUMMARY OF THE INVENTION

The object of the present invention is to provide a chimeric antigen receptor that can increase anti-tumor efficacy and cytokine secretion ability and use thereof.

In order to achieve the above object, the present invention provides a chimeric antigen receptor including: a target antigen binding domain; a transmembrane domain; a CD30-derived intracellular signaling domain including one or more amino acid sequences selected from the group consisting of SEQ ID NOs: 44, 46, 48 and 50; and a CD3ζ intracellular signaling domain.

The present invention also provides a nucleic acid molecule encoding the chimeric antigen receptor.

The present invention also provides a vector including the nucleic acid molecule.

The present invention also provides an isolated immune effector cell including the chimeric antigen receptor, a nucleic acid molecule encoding the same, or a vector including the nucleic acid molecule.

The present invention also provides an anti-tumor composition including the immune effector cell.

The present invention also provides a method of treating cancer, including administering a therapeutically effective amount of the immune effector cell to a subject in need thereof.

The present invention has an effect of enhancing antitumor efficacy and cytokine secretion ability by increasing the proliferation and survival of immune effector cells by using a chimeric antigen receptor including a partial sequence of the TRAF binding site in a CD30 domain as an intracellular signaling domain.

### BRIEF DESCRIPTION OF THE DRAWINGS

FIG. 1a shows a schematic diagram of CD19-28-z, CD19-30L-z, CD19-30M-z, CD19-30M^{mut}_z, CD19-30ΔM-z, and CD19-30S-z CAR structures according to an embodiment of the present invention.

FIG. 1b shows the expression level of human CD19 antigen (red area) in human blood cancer cell lines U937^{mock}, U937CD19, IM-9, and Raji, analyzed through flow cytometry based on isotype (gray area) according to an embodiment of the present invention.

FIG. 1c shows the expression level of each CAR (red area) in KHYG-1, NK-92, αβ T, and γδ T cells into which CD19-28-z, CD19-30L-z, CD19-30M-z, CD19-30M^{mut}-z, CD19-30ΔM-z, and CD19-30S-z CARs are introduced, analyzed through flow cytometry based on a group (NT, blue area) in which a CAR is not introduced according to an embodiment of the present invention.

FIG. 1d shows the cytolytic activity against CD19 positive and negative human blood cancer cell lines for each of KHYG-1, NK-92, αβ T, and γδ T cells into which CD19-28-z, CD19-30L-z, CD19-30M-z, CD19-30M^{mut}-z, CD19-30ΔM-z, and CD19-30S-z CARs are introduced at various E/T ratios according to an embodiment of the present invention.

FIG. 2a shows a schematic diagram of CD19-30S-z, CD19-30ΔS-z, CD19-30ΔS^{YN}-z, and CD19-30ΔS^{YF}-z CAR structures according to an embodiment of the present invention.

FIG. 2b shows the expression level of each CAR (red area) in αβ T and γδ T cells into which CD19-28-z, CD19-30L-z, CD19-30S-z, and CD19-30ΔS-z CARs are introduced, analyzed through flow cytometry based on a group (NT, blue area) in which a CAR is not introduced according to an embodiment of the present invention.

FIG. 2c shows the cytolytic activity against CD19 positive and negative human blood cancer cell lines for each of αβ T and γδ T cells into which CD19-28-z, CD19-30L-z, CD19-30S-z, and CD19-30ΔS-z CARs are introduced at various E/T ratios according to an embodiment of the present invention.

FIG. 2d shows the expression level of each CAR (red area) in αβ T and γδ T cells into which CD19-28-z, CD19-30S-z, CD19-30ΔS^{YN}-z, and CD19-30Δ^{YF}-z CARs are introduced, analyzed through flow cytometry based on a group (NT, blue area) in which a CAR is not introduced according to an embodiment of the present invention.

FIG. 2e shows the cytolytic activity against CD19 positive and negative human blood cancer cell lines for each of αβ T and γδ T cells into which CD19-28-z, CD19-30S-z, CD19-30ΔS^{YN}-z, and CD19-30Δ^{YF}-z CARs are introduced at various E/T ratios according to an embodiment of the present invention.

FIG. 3a shows a schematic diagram of CD19-28-z, CD19-ICOS-z, CD19-4-1BB-z, CD19-OX40-z, CD19-27-z, and CD19-30S-z CAR structures according to an embodiment of the present invention.

FIG. 3b shows the expression level of each CAR (red area) in αβ T and γδ T cells into which CD19-28-z, CD19-ICOS-z, CD19-4-1BB-z, CD19-OX40-z, CD19-27-z, and CD19-30S-z CARs are introduced, analyzed through flow cytometry based on a group (NT, blue area) in which a CAR is not introduced according to an embodiment of the present invention.

FIG. 3c shows the cytolytic activity against CD19 positive and negative human cancer cell lines for each of αβ T and γδ T cells into which Ig receptor family CD19-28-z and CD19-ICOS-z CARs are introduced at various E/T ratios along with the cytolytic activity for each of αβ T and γδ T cells into which a CD19-30S-z CAR is introduced according to an embodiment of the present invention.

FIG. 3d shows the cytolytic activity against CD19 positive and negative blood cancer cell lines for each of αβ T and γδ T cells into which CD19-4-1BB-z, CD19-OX40-z, and CD19-27-z CARs of the TNFR family are introduced at various E/T ratios along with the cytolytic activity for each of αβ T and γδ T cells into which a CD19-30S-z CAR is introduced according to an embodiment of the present invention.

FIG. 3e shows the secretion amount of cytokines INF-γ, TNF-α, granzyme A, granzyme B, and perforin at an E/T ratio of 1 for CD19 negative U937^{mock} and positive U937CD19 in αβ T cells and γδ T cells into which CD19-28-z, CD19-4-1BB-z, and CD19-30S-z CARs are introduced according to an embodiment of the present invention.

FIG. 4a shows a schematic diagram of CD19-28-BB-z, CD19-30S-BB-z, CD19-28-30S-z, CD19-30S-ICOS-z, CD19-30S-OX40-z, and CD19-30S-27-z CAR structures according to an embodiment of the present invention.

FIG. 4b shows the expression level of each CAR (red area) in αβ T and γδ T cells into which CD19-28-BB-z, CD19-30S-BB-z, CD19-28-30S-z, CD19-30S-ICOS-z, CD19-30S-OX40-z, and CD19-30S-27-z CARs are introduced, analyzed through flow cytometry based on a group (NT, blue area) in which a CAR is not introduced according to an embodiment of the present invention.

FIG. 4c shows the cytolytic activity against CD19 positive and negative human blood cancer cell lines for each of αβ T and γδ T cells into which CD19-28-BB-z, CD19-30S-BB-z, CD19-28-30S-z, CD19-30S-ICOS-z, CD19-30S-OX40-z, and CD19-30S-27-z CARs are introduced at various E/T ratios according to an embodiment of the present invention.

FIG. 4d shows the secretion amount of cytokines INF-γ, TNF-α, granzyme A, granzyme B, and perforin at an E/T ratio of 1 for CD19 negative U937^{mock} and positive U937CD19 in αβ T cells and γδ T cells into which CD19-28-BB-z, CD19-30S-BB-z, CD19-28-30S-z, CD19-30S-ICOS-z, CD19-30S-OX40-z, and CD19-30S-27-z CARs are introduced according to an embodiment of the present invention.

FIG. 5a shows a schematic diagram of BCMA-28-BB-z, BCMA-30S-BB-z, and BCMA-28-30S-z CAR structures according to an embodiment of the present invention.

FIG. 5b shows the expression level of human BCMA antigen (red area) in human blood cancer cell lines U937, IM-9, and Daudi based on the isotype (gray area), analyzed through flow cytometry according to an embodiment of the present invention.

FIG. 5c shows the expression level of each CAR (red area) in γδ T cells into which BCMA-28-BB-z, BCMA-30S-BB-z, and BCMA-28-30S-z CARs are introduced, analyzed through flow cytometry based on a group (NT, blue area) in which a CAR is not introduced according to an embodiment of the present invention.

FIG. 5d shows the cytolytic activity against BCMA positive and negative human cancer cell lines for γδ T cells into which BCMA-28-BB-z, BCMA-30S-BB-z, and BCMA-28-30S-z CARs are each introduced at various E/T ratios according to an embodiment of the present invention.

FIG. 6a shows a schematic diagram of EpCAM-28-z, EpCAM-4-1BB-z, EpCAM-30S-z, and EpCAM-28TM CAR structures according to an embodiment of the present invention.

FIG. 6b shows the expression level of human EpCAM antigen (red area) in human lung cancer cell lines A549, Calu-1, NCI-H292, and NCI-H460 based on the isotype (gray area), analyzed through flow cytometry according to an embodiment of the present invention.

FIG. 6c shows the expression level of each CAR (red area) in αβ T and γδ T cells into which EpCAM-28-z, EpCAM-4-1BB-z, EpCAM-30S-z, and EpCAM-28TM CARs are introduced, analyzed through flow cytometry based on a group (NT, blue area) in which a CAR is not introduced according to an embodiment of the present invention.

FIG. 6d shows the cytolytic activity against an EpCAM positive human lung cancer cell line for each of αβ T and γδ T cells into which EpCAM-28-z, EpCAM-4-1BB-z, EpCAM-30S-z, and EpCAM-28TM CARs are introduced at various E/T ratios according to an embodiment of the present invention.

FIG. 6e shows a schematic diagram of EpCAM-28-BB-z, EpCAM-30S-BB-z, and EpCAM-28-30S-z CAR structures according to an embodiment of the present invention.

FIG. 6f shows the expression level of each CAR (red area) in αβ T and γδ T cells into which EpCAM-28-BB-z, EpCAM-30S-BB-z, and EpCAM-28-30S-z CARs are introduced, analyzed through flow cytometry based on a group (NT, blue area) in which a CAR is not introduced according to an embodiment of the present invention.

FIG. 6g shows the cytolytic activity against an EpCAM positive human lung cancer cell line for each of αβ T and γδ T cells into which EpCAM-28-BB-z, EpCAM-30S-BB-z, and EpCAM-28-30S-z CARs are introduced at various E/T ratios according to an embodiment of the present invention.

FIG. 7a shows a schematic diagram of MSLN-28-z, MSLN-4-1BB-z, and MSLN-30S-z CAR structures according to an embodiment of the present invention.

FIG. 7b shows the expression level of human MSLN antigen (red area) in human lung cancer cell lines NCI-H292 and A549, a human pancreatic cancer cell line AsPC-1, and a human ovarian cancer cell line SKOV3 based on the isotype (gray area), analyzed through flow cytometry according to an embodiment of the present invention.

FIG. 7c shows the expression level of each CAR (red area) in αβ T and γδ T cells into which MSLN-28-z, MSLN-4-1BB-z, and MSLN-30S-z CARs are introduced, analyzed through flow cytometry based on a group (NT, blue area) in which a CAR is not introduced according to an embodiment of the present invention.

FIG. 7d shows the cytolytic activity against MSLN positive and negative human cancer cell lines for each of αβ T and γδ T cells into which MSLN-28-z, MSLN-4-1BB-z, and MSLN-30S-z CARs are introduced at various E/T ratios according to an embodiment of the present invention.

FIG. 7e shows a schematic diagram of MSLN-28-BB-z, MSLN-30S-BB-z, and MSLN-28-30S-z CAR structures according to an embodiment of the present invention.

FIG. 7f shows the expression level of each CAR (red area) in αβ T and γδ T cells into which MSLN-28-BB-z, MSLN-30S-BB-z, and MSLN-28-30S-z CARs are introduced, analyzed through flow cytometry based on a group (NT, blue area) in which a CAR is not introduced according to an embodiment of the present invention.

FIG. 7g shows the cytolytic activity against MSLN positive and negative human cancer cell lines for each of αβ T and γδ T cells into which MSLN-28-BB-z, MSLN-30S-BB-z, and MSLN-28-30S-z CARs are introduced at various E/T ratios according to an embodiment of the present invention.

FIG. 8a shows a schematic diagram of GPC3-28-BB-z, GPC3-30S-BB-z, and GPC3-28-30S-z CAR structures according to an embodiment of the present invention.

FIG. 8b shows the expression level of human GPC3 antigen (red area) in a human liver cancer cell line HepG2 and human lung cancer cell lines NCI-H292 and A549 based on the isotype (gray area), analyzed through flow cytometry according to an embodiment of the present invention.

FIG. 8c shows the expression level of each CAR (red area) in γδ T cells into which GPC3-28-BB-z, GPC3-30S-BB-z, and GPC3-28-30S-z CARs are introduced, analyzed through flow cytometry based on a group (NT, blue area) in which a CAR is not introduced according to an embodiment of the present invention.

FIG. 8d shows the cytolytic activity against each of GPC3 positive and negative human cancer cell lines for γδ T cells into which GPC3-28-BB-z, GPC3-30S-BB-z, and GPC3-28-30S-z CARs are introduced at various E/T ratios according to an embodiment of the present invention.

FIG. 9a shows a schematic diagram of PD-1-28-z, PD-1-4-1BB-z, PD-1-30S-z, and PD-1-28TM CAR structures according to an embodiment of the present invention.

FIG. 9b shows the expression level of human PD-L1 antigen (red area) in the human pancreatic cancer cell line AsPC-1, human lung cancer cell lines NCI-H292 and Calu-1, and a human head and neck cancer cell line FaDu based on the isotype (gray area), analyzed through flow cytometry according to an embodiment of the present invention.

FIG. 9c shows the expression level of each CAR (red area) in αβ T and γδ T cells into which PD-1-28-z, PD-1-4-1BB-z, PD-1-30S-z, and PD-1-28TM CARs are introduced, analyzed through flow cytometry based on a group (NT, blue area) in which a CAR is not introduced according to an embodiment of the present invention.

FIG. 9d shows the cytolytic activity against each of PD-L1 positive and negative human cancer cell lines for each of αβ T and γδ T cells into which PD-1-28-z, PD-1-4-1BB-z, PD-1-30S-z, and PD-1-28TM CARs are introduced at various E/T ratios according to an embodiment of the present invention.

FIG. 9e shows a schematic diagram of PD-1-28-BB-z, PD-1-30S-BB-z, and PD-1-28-30S-z CAR structures according to an embodiment of the present invention.

FIG. 9f shows the expression level of each CAR (red area) in γδ T cells into which PD-1-28-BB-z, PD-1-30S-BB-z, and PD-1-28-30S-z CARs are each introduced, analyzed through flow cytometry based on a group (NT, blue area) in which a CAR is not introduced according to an embodiment of the present invention.

FIG. 9g shows the cytolytic activity against each of PD-L1 positive and negative human cancer cell lines for γδ T cells into which PD-1-28-BB-z, PD-1-30S-BB-z, and PD-1-28-30S-z CARs are each introduced at various E/T ratios according to an embodiment of the present invention.

FIG. 10a shows a schematic diagram of NKp30-28-BB-z, NKp30-30S-BB-z, and NKp30-28-30S-z CAR structures according to an embodiment of the present invention.

FIG. 10b shows the expression level of human B7-H6 antigen (red area) in human blood cancer cell lines U937, Raji, and K562 and the human lung cancer cell line A549 based on the isotype (gray area), analyzed through flow cytometry according to an embodiment of the present invention.

FIG. 10c shows the expression level of each CAR (red area) in αβ T and γδ T cells into which NKp30-28-BB-z, NKp30-30S-BB-z, and NKp30-28-30S-z CARs are introduced, analyzed through flow cytometry based on a group (NT, blue area) in which a CAR is not introduced according to an embodiment of the present invention.

FIG. 10d shows the cytolytic activity against each of B7-H6 positive and negative human cancer cell lines for each of αβ T and γδ T cells into which NKp30-28-BB-z, NKp30-30S-BB-z, and NKp30-28-30S-z CARs are introduced at various E/T ratios according to an embodiment of the present invention.

FIGS. 11a and 11b show the therapeutic anti-tumor results of second-generation CD19-CAR-expressing T cells according to the combination between CD30S and an existing ICD (CD28, 4-1BB) in an animal model xenografted with human leukemia cell line NALM6. FIG. 11a shows the tumor growth over time in the CD19-28-z, CD19-4-1BB-z, and CD19-30S-z CAR-αβ T cell treatment groups according to an embodiment of the present invention, and FIG. 11b shows the survival rate of the mice up to 48 days in the control group and the CD19-28-z, CD19-4-1BB-z, and CD19-30S-z CAR-αβ T cell treatment groups. Tumor growth was monitored through in vivo bioluminescence imaging in individual mice and quantified as the average luminescence of bioluminescence.

### DETAILED DESCRIPTION OF EXEMPLARY EMBODIMENTS

Hereinafter, the features of the present invention will be described in detail.

The present invention relates to a chimeric antigen receptor including: a target antigen binding domain; a transmembrane domain; a CD30-derived intracellular signaling domain including one or more amino acid sequences selected from the group consisting of SEQ ID NOs: 44, 46, 48 and 50; and a CD3ζ intracellular signaling domain.

The present invention is characterized in that it provides a chimeric antigen receptor including a partial sequence of a TNF receptor-associated factor (TRAF) binding site in a CD30 domain as an intracellular signaling domain.

The CD30, a 120 kD transmembrane glycoprotein receptor, is a member of the tumor necrosis factor receptor (TNFR) family and is expressed in T cells and B cells. When activated, CD30 recruits TNFR associated factors (TRAF1, 2, and 5) to activate the JUNB or AP-1 transcription factor through the NF-κB or MAPK signaling pathway. Through this, not only the proliferation and survival of T cells increase, but also their phenotype is differentiated into a memory type. Therefore, to increase the proliferation and survival of immune effector cells, especially CAR-T cells, by using the signal transmitted by TRAF, the present inventors produced various types of CD30 domains (CD30L, CD30M, CD30M^{mut}, CD30M, CD30S) focusing on TRAF binding sites (domains 1, 2, and 3) of the CD30 domain, and as a result, confirmed that the anti-tumor efficacy and cytokine secretion ability were improved when the 57 a.a. region of 539-595 a.a. or 544-588 a.a. of domain 2+3 was used as an intracellular signaling domain of the chimeric antigen receptor.

As the intracellular signaling domain of the chimeric antigen receptor of the present invention, in addition to the 539-595 a.a. or 544-588 a.a. region of domain 2+3, CD30ΔS, which further includes specific amino acids, YMNM or YMFM, that is, 544-588 a.a.+YMNM or YMFM is used. These amino acid sequences are sequences that are not present in wild-type CD30 and are phosphoinositide 3-kinase binding site sequences, and according to one embodiment, the anti-tumor efficacy and cytokine secretion ability of immune effector cells were similar, compared to the case where only the 539-595 a.a. or 544-588 a.a. region of domain 2+3 of CD30 was used for the chimeric antigen receptor.

Therefore, the chimeric antigen receptor of the present invention is characterized in that it significantly increases the anti-tumor efficacy and cytokine secretion ability of immune effector cells by using an intracellular signaling domain including the 539-595 a.a. region or 544-588 a.a. region of domain 2+3 of CD30S or 544-588 a.a.+a YMNM or YMFM sequence.

The term “chimeric antigen receptor (CAR)” used in the present invention generally refers to a fusion protein containing an extracellular domain that has the ability to bind to an antigen and one or more intracellular domains. A CAR may include an antigen (e.g., surface antigen, tumor-associated antigen, etc.) binding domain, a transmembrane domain, and an intracellular signaling domain. A CAR may be combined with T cell receptor-activating intracellular domains based on target antigen specificity. Genetically modified CAR-expressing T cells may specifically identify and eliminate target antigen-expressing malignant cells.

The term “target antigen binding domain” used in the present invention generally refers to a domain having the ability to specifically bind to an antigen protein. For example, it may be an antibody or a fragment thereof that specifically binds to a target antigen.

The term “binding domain” used in the present invention may be used interchangeably with “extracellular domain,” “extracellular binding domain,” “antigen-specific binding domain,” and “extracellular antigen-specific binding domain,” and it refers to a CAR domain or fragment that has the ability to specifically bind to a target antigen.

The target antigen may be a surface antigen or a tumor-associated antigen expressed in hematological cancer or solid cancer. For example, the antigen may be selected from CD19, MUC16, MUC1, CAIX, CEA, CDS, CD7, CD10, CD20, CD22, CD30, CD33, CD34, CD38, CD41, CD44, CD49f, CD56, CD74, CD133, CD138, BCMA, PD-1, NKp30, cytomegalovirus (CMV) infected cell antigen, EGP-2, EGP-40, EpCAM, Erb-B2, Erb-B3, Erb-B4, FBP, fetal acetylcholine receptor, folate receptor-α, GD2, GD3, HER-2, hTERT, IL-13R-a2, K-light chain, KDR, LeY, L1 cell adhesion molecule, MAGE-A1, mesothelin, NKG2D ligand, NY-ESO-1, carcinoembryonic antigen (h5T4), PSCA, PSMA, ROR1, TAG-72, VEGF-R2, WT-1, CD24, CD47, EGFR, CD123, GPC3, CD117, Claudin18.2, cMET, EphA2, CLL-1, CD171, AXL, ROR2, FAP or CD5, but is not limited thereto.

In the present invention, the antibody that specifically binds to a target antigen may be a monoclonal antibody. The term “monoclonal antibody” used in the present invention is an antibody produced by a single antibody-forming cell and is characterized by a uniform primary structure (amino acid sequence). A monoclonal antibody recognizes only one antigenic determinant and is generally produced by culturing a hybridoma cell that is a fusion of a cancer cell and an antibody-producing cell, but it can also be produced by using other recombinant protein-expressing host cells using a secured antibody gene sequence.

The term “antibody” used in the present invention may be used not only in its complete form, which has two full-length light chains and two full-length heavy chains, but also in fragments of the antibody molecule. A fragment of an antibody molecule refers to a fragment that possesses at least a peptide tag (epitope) binding function and includes scFv, Fab, F(ab′), F(ab′) 2, a single domain, and the like.

Among antibody fragments, Fab is a structure that has variable regions of the light and heavy chains, a constant region of the light chain, and a first constant region (CH1) of the heavy chain, and it has one antigen binding site. Fab′ differs from Fab in that it has a hinge region including one or more cysteine residues at the C terminus of the heavy chain CH1 domain. An F(ab′) 2 antibody is produced when the cysteine residue in the hinge region of Fab′ forms a disulfide bond. Fv is a minimal antibody fragment having only the heavy chain variable region and the light chain variable region. The recombinant technology for producing an Fv fragment is disclosed in international patents WO 88/10649, WO 88/106630, WO 88/07085, WO 88/07086, and WO 88/09344. In a disulfide-stabilized Fv (dsFv), the heavy chain variable region and the light chain variable region are connected by a disulfide bond, and in single-chain Fv (scFv), the heavy chain variable region and the light chain variable region are generally connected by a covalent bond through a peptide linker. These antibody fragments may be obtained using proteolytic enzymes (for example, Fab may be obtained by restriction digestion of an entire antibody with papain, and an F(ab′) 2 fragment may be obtained by digestion with pepsin). Preferably, they may be produced through genetic recombination technology.

The term “humanized antibody” used in the present invention is an antibody that possesses an amino acid sequence corresponding to that of an antibody produced by humans and/or has been produced using one of the techniques for producing human antibodies as disclosed in the present invention. This definition of humanized antibody specifically excludes humanized antibodies that include a non-human antigen-binding moiety.

In addition, the protein, polypeptide and/or amino acid sequence included in the present invention should be understood as including at least functional variants or homologs having the same or similar function as the protein or polypeptide.

In the present invention, functional variants may be proteins or polypeptides obtained by substituting, deleting, or adding one or more amino acids in the amino acid sequence of the protein and/or polypeptide. For example, functional variants may include proteins or polypeptides having different amino acid sequences due to substitution, deletion and/or insertion of one or more amino acids such as 1 to 30, 1 to 20 or 1 to 10 or 1, 2, 3, 4 or 5 amino acids. Functional variants may substantially maintain the biological properties of an unmodified protein or polypeptide (substitution, deletion or addition). For example, functional variants may retain at least 60%, 70%, 80%, 90%, or 100% of the biological activity (such as antigen-binding ability) of original proteins or polypeptides.

In the present invention, a homolog may be a protein or polypeptide (e.g., an antibody capable of specifically binding to BCMA or a fragment thereof) having about 85% or more amino acid sequence homology with the protein and/or polypeptide (e.g., about 85%, 90%, 91%, 92%, 93%, 94%, 95%, 96%, 97%, 98%, 99% or more).

In the present invention, homology generally refers to similarity or correlation between two or more sequences.

The term “transmembrane domain” used in the present invention generally refers to a CAR domain that plays a role in signaling by passing through the cell membrane and being connected to an intracellular signaling domain. The transmembrane domain is connected between the C-terminus of the target antigen binding domain and the N-terminus of the intracellular signaling domain and may be a transmembrane domain of a TCR co-receptor or T cell costimulatory molecule selected from the group consisting of CD8, 4-1BB, CD27, CD28, CD30, 0X40, CD3e, CD3ζ, CD45, CD4, CD5, CD9, CD16, CD22, CD33, CD37, CD64, CD80, CD86, CD134, CD137, CD154, and ICOS (CD278), or may be derived therefrom. Specifically, a transmembrane domain of CD28 may be used.

A hinge region may be connected between the C-terminus of the target antigen binding domain and the N-terminus of the transmembrane domain, and as the hinge region, one that is derived from CD8α or CD28 may be used. The “hinge region” generally refers to a connecting region between an antigen-binding region and an immune cell Fc receptor (FcR)-binding region.

The term “intracellular signaling domain” (or intracellular signal transduction domain) used in the present invention refers to a domain that is generally positioned inside a cell and is capable of transmitting signals. In the present invention, not only 539-595 a.a. of domain 2+3 among the TRAF binding sites of the CD30 region or its deletion site, 544-588 a.a., but also CD30S, which further includes specific amino acids, YMNM or YMFM, in addition to 544-588 a.a., may be used as an intracellular signaling domain. More specifically, an amino acid sequence of SEQ ID NO: 44, 46, 48 or 50 may be included.

As a CAR of the present invention, a third-generation chimeric antigen receptor further including an intracellular signaling domain (or co-stimulatory domain) selected from the group consisting of CD27, CD28, 4-1BB (CD137), 0X40, CD40, ICOS, lymphocyte function-associated antigen-1 (LFA-1), CD2, CD7, NKG2C, CD83, Dap10, GITR, OX40L, Myd88-CD40, and KIR2DS2 may be used.

The CD3ζ intracellular signaling domain of the CAR of the present invention may have the amino acid sequence of SEQ ID NO: 18, which corresponds to CD32-derived nucleotides 154-492 (NCBI NM_198053.2), but is not limited thereto.

In addition, the CAR of the present invention may further include a signal peptide at the N-terminus of a target antigen binding domain, and the “signal peptide” generally refers to a peptide chain for guiding protein delivery. As the signal peptide, a granulocyte macrophage-colony stimulating factor (GM-CSF) receptor signal sequence, a CD8α signal sequence, an immunoglobulin heavy chain signal sequence, a PD-1 signal sequence, and an NKp30 signal sequence may be used, but it is not limited thereto.

According to one embodiment of the present invention, the CAR of the present invention may be a second-generation CAR in which a signal peptide; a target antigen binding domain; a hinge domain; a transmembrane domain; a CD30-derived intracellular signaling domain; and a CD3ζ intracellular signaling domain are sequentially linked.

According to another embodiment of the present invention, the CAR of the present invention may be a third-generation CAR in which a signal peptide; a target antigen binding domain; a hinge domain; a transmembrane domain; a CD30-derived intracellular signaling domain; an intracellular signaling domain (costimulatory domain) selected from the group consisting of CD27, CD28, 4-1BB (CD137), 0X40, CD40, ICOS, LFA-1, CD2, CD7, NKG2C, CD83, Dap10, GITR, OX40L, Myd88-CD40, and KIR2DS2; and a CD3ζ intracellular signaling domain are sequentially linked.

The present invention also relates to a nucleic acid molecule encoding the CAR.

The nucleic acid molecule encoding the CAR may include polynucleotides each encoding a signal peptide; a target antigen binding domain; a hinge domain; a transmembrane domain; a CD30-derived intracellular signaling domain; and a CD32 intracellular signaling domain.

The nucleic acid molecule encoding the CAR may include polynucleotides each encoding a signal peptide; a target antigen binding domain; a hinge domain; a transmembrane domain; a CD30-derived intracellular signaling domain; an intracellular signaling domain (costimulatory domain) selected from the group consisting of CD27, CD28, 4-1BB (CD137), 0X40, CD40, ICOS, LFA-1, CD2, CD7, NKG2C, CD83, Dap10, GITR, OX40L, Myd88-CD40, and KIR2DS2; and a CD3ζ intracellular signaling domain

More specifically, the CD30-derived intracellular signaling domain may include a base sequence of SEQ ID NO: 47 or 49.

In the present invention, the term “polynucleotide” generally refers to a nucleic acid molecule, deoxyribonucleotides or ribonucleotides, or an analog thereof, separated in any length. For example, the polynucleotide of the present invention may be produced through (1) in vitro amplification, such as polymerase chain reaction (PCR) amplification; (2) cloning and recombination; (3) purification such as digestion and gel electrophoresis separation; (4) synthesis such as chemical synthesis, and preferably, an isolated polynucleotide is produced by recombinant DNA technology. In the present invention, a nucleic acid for encoding an antibody or an antigen-binding fragment thereof may be produced by various methods known in the art, including restriction fragment manipulation of synthetic oligonucleotides or application of splicing by overlap extension (SOE) PCR but not limited thereto.

The present invention also relates to a vector including a nucleic acid molecule encoding the CAR.

The term “expression vector” used in the present invention refers to a gene product including essential regulatory elements such as a promoter to enable expression of a target gene in an appropriate host cell. The vector may be selected from one or more of plasmids, retroviral vectors, and lentiviral vectors. Once transformed into a suitable host, the vector may replicate and function independently of the host genome, or in some cases it may be integrated into the genome itself.

In addition, vectors may include expression regulatory elements that allow a coding region to be expressed correctly in a suitable host. These regulatory elements are well known to those skilled in the art and may include, for example, a promoter, a ribosome-binding site, an enhancer, and other regulatory elements to regulate gene transcription or mRNA translation. The specific structure of an expression regulatory sequence may vary depending on the function of the species or cell type, but it generally contains a 5′ non-transcribed sequence and a 5′ or 3′ non-translated sequence that participate in transcription initiation and translation initiation, respectively, such as the TATA box, capped sequence, CAAT sequence, and the like. For example, a 5′ non-transcribed expression regulatory sequence may include a promoter region, which may include a promoter sequence for transcribing a functionally linked nucleic acid and regulating the transcription thereof.

The promoter is operably linked to induce the expression of a target antigen binding domain, where “operably linked” means that a nucleic acid expression regulatory sequence is functionally linked with a nucleic acid sequence encoding a protein of interest to perform a general function. An operable link with a recombinant vector may be produced using genetic recombination techniques well known in the art, and site-specific DNA cleavage and ligation may be achieved using enzymes generally known in the art Methods for introducing and expressing a gene into cells are known in the art. With regard to expression vectors, a vector may be easily introduced into host cells by any method in the art. For example, an expression vector may be introduced into host cells by physical, chemical, or biological means.

Physical methods for introducing a polynucleotide into host cells include calcium phosphate precipitation, lipofection, particle bombardment, microinjection, and electroporation. Methods for producing cells including vectors and/or exogenous nucleic acids are well known in the art. See, for example, Sambrook et al., 2012, MOLECULAR CLONING: A LABORATORY MANUAL, volumes 1-4, Cold Spring Harbor Press, NY.

Biological methods for introducing a polynucleotide into host cells include the use of DNA and RNA vectors. Viral vectors, and especially retroviral vectors, have become the most widely used method for inserting genes into mammalian cells, for example, human cells. Other viral vectors may be derived from lentiviruses, poxviruses, herpes simplex viruses, adenoviruses, adeno-associated viruses, and the like.

Chemical means for introducing a polynucleotide into host cells include colloidal dispersion systems, such as macromolecular complexes, nanocapsules, microspheres, and beads, and lipid-based systems including oil-in-water emulsions, micelles, mixed micelles, and liposomes. An exemplary colloidal system for use as delivery vehicles in vitro and in vivo is a liposome (e.g., artificial membrane vesicle). Other methods are available for the state-of-the-art targeted delivery of nucleic acids, such delivery of polynucleotides using targeted nanoparticles or other suitable as submicrometer-sized delivery systems.

When non-viral delivery systems are used, an exemplary delivery vehicle is a liposome. The use of lipid preparations is considered for the introduction of nucleic acids into host cells (in vitro, ex vivo or in vivo). In another aspect, nucleic acids may be associated with lipids. Nucleic acids associated with lipids may be encapsulated within the aqueous interior of liposomes, interspersed within the lipid bilayer of liposomes, attached to liposomes via linkage molecules associated with both the liposomes and the oligonucleotides, trapped within liposomes, complexed with liposomes, dispersed in a lipid-containing solution, mixed with a lipid, combined with a lipid, contained within a lipid as a suspension, contained or complexed with micelles, or otherwise associated with a lipid. The lipid, lipid/DNA or lipid/expression vector association composition is not limited to any particular structure in a solution.

The present invention also relates to an immune effector cell including the CAR, a nucleic acid molecule encoding the same, or a vector including the nucleic acid molecule.

The immune effector cells may be mammalian-derived cells, preferably αβ T cells, γδ T cells, NK cells (including KHYG-1 and NK-92 cell lines), NK T cells, or macrophages.

The immune effector cells expressing the CAR may be produced by introducing the CAR vector of the present invention into immune effector cells, such as T cells or NK cells.

Specifically, a CAR vector may be introduced into cells by methods known in the art, such as electroporation and Lipofectamine (Lipofectamine 2000, Invitrogen Corporation). For example, a plasmid may be introduced into immune effector cells by electroporation to ensure long-term and stable CAR expression.

Immune effector cells for producing immune effector cells expressing a CAR may be obtained from a subject, where the “subject” includes a living organism (e.g., a mammal) against which an immune response may be elicited. Examples of the subject include humans, dogs, cats, mice, rats, and transformants thereof. T cells may be obtained from numerous sources, including peripheral blood mononuclear cells, bone marrow, lymph node tissue, umbilical cord blood, thymic tissue, tissue from a site of infection, ascites, pleural effusion, spleen tissue, and tumors.

The T cells may be obtained from blood units collected from a subject using any of a number of techniques known to those skilled in the art, for example, Ficoll® separation. Cells from blood are obtained by apheresis, and the apheresis product typically contains lymphocytes, including T cells, monocytes, granulocytes, and B cells, other nucleated white blood cells, red blood cells, and platelets.

Cells collected by apheresis may be washed to remove the plasma fraction and placed in an appropriate buffer or medium for subsequent processing steps. T cells are isolated from peripheral blood lymphocytes by lysing red blood cells and depleting monocytes, for example, by centrifugation through a PERCOLL® gradient or by countercurrent centrifugation.

The present invention also relates to an anti-tumor composition including the above immune effector cells.

According to one embodiment of the present invention, the immune effector cells into which a CAR including a CD30-derived intracellular signaling domain has been introduced exhibits specific cytolytic ability against a CD19 positive blood cancer cell line, a BCMA positive cell line, an EpCAM solid tumor cell line, a mesothelin positive solid cancer cell line, a GPC3 positive solid cancer cell lines, a PD-L1 positive solid cancer cell line, a B7-H6 positive cell line, and the like. Therefore, the immune effector cells into which the CAR of the present invention has been introduced may be used to treat blood cancer or solid cancer.

The solid cancer may be lung cancer, colorectal cancer, prostate cancer, thyroid cancer, breast cancer, brain cancer, head and neck cancer, esophageal cancer, skin cancer, melanoma, retinoblastoma, thymus cancer, stomach cancer, colon cancer, liver cancer, ovarian cancer, uterine cancer, bladder cancer, rectal cancer, gallbladder cancer, biliary tract cancer, or pancreatic cancer. In addition, the blood cancer may be lymphoma, leukemia, or multiple myeloma.

The pharmaceutical composition may further include a pharmaceutically acceptable carrier. For oral administration, binders, lubricants, disintegrants, excipients, solubilizers, dispersants, stabilizers, suspending agents, colorants, flavorings, and the like may be used, and in the case of injections, buffers, preservatives, analgesics, solubilizers, and isotonic agents, stabilizers, and the like may be mixed and used, and in the case of topical administration, bases, excipients, lubricants, preservatives, and the like may be used.

The formulation of the pharmaceutical composition may be prepared in various forms by mixing it with the above-described pharmaceutically acceptable carrier. For example, for oral administration, the pharmaceutical composition may be prepared in the form of tablets, troches, capsules, elixirs, suspension, syrup, wafers, and the like, and in the case of injections, it may be prepared in the form of unit dosage ampoules or multiple dosage forms.

In addition, the pharmaceutical composition may include a surfactant that can improve membrane permeability. The surfactant may be derived from steroids or may be cationic lipids such as N-[1-(2,3-dioleoyl)propyl-N,N,N-trimethylammonium chloride (DOTMA), or various compounds such as cholesterol hemisuccinate and phosphatidyl glycerol, but is not limited thereto.

The pharmaceutical composition may be administered together with or sequentially with the above-described pharmacological or physiological components and may also be administered in combination with additional conventional therapeutic agents and may be administered sequentially or simultaneously with conventional therapeutic agents. Such administration may be single or multiple administrations. It is important to administer an amount that can achieve the maximum effect with the minimum amount without side effects by considering all of the above factors, and this may be easily determined by those skilled in the art.

The term “administration” used in the present invention means providing a pharmaceutical composition of the present invention to a subject by any suitable method.

The pharmaceutical composition of the present invention may be administered in an amount of an active ingredient or pharmaceutical composition that induces a biological or medical response in a tissue system, animal, or human, as considered by a researcher, veterinarian, physician, or other clinicians, that is, a therapeutically effective amount that induces alleviation of the symptoms of a disease or disorder being treated. It is obvious to those skilled in the art that the therapeutically effective dosage and the number of times of administration of the pharmaceutical composition of the present invention will vary depending on the desired effect. Therefore, the optimal dosage to be administered may be easily determined by those skilled in the art, and it may be adjusted depending on various factors including the type of disease, the severity of the disease, the content of the active ingredient and other ingredients contained in the composition, the type of dosage form, the patient's age, weight, general health conditions, gender, and diet, administration time, administration route, secretion rate of the composition, treatment period, and concurrently used drugs. The pharmaceutical composition of the present invention may be administered in an amount of 1 to 10,000 mg/kg/day and may be administered once a day or in several divided doses.

The term “subject” used in the present specification refers to a mammal suffering from or at risk of a condition or disease that may be alleviated, suppressed, or treated by administering the pharmaceutical composition, and preferably, it refers to a human.

Therefore, the present invention also provides a method of treating cancer, including administering a therapeutically effective amount of immune effector cells to a subject in need thereof.

The immune effector cells may be administered in a pharmaceutically effective amount to treat cancer expressing a tumor antigen. The pharmaceutically effective amount may vary depending on various factors including the type of disease, the patient's age and weight, the nature and severity of symptoms, the type of current treatment, number of treatments, and administration form and route, and it may be easily determined by experts in the field.

The subject is the same as the subject to which the pharmaceutical composition of the present invention is administered.

Hereinafter, the present invention will be described in more detail through examples according to the present invention, but the scope of the present invention is not limited to the examples presented below.

### EXAMPLES

### <Example 1> Production of CARs

### (Human Cell Lines)

Human multiple myeloma cell line IM-9, human lymphoma cell lines U937^{mock} and U937CD19, human Burkitt's lymphoma cell line Raji, human lung cancer cell lines NCI-H292 and NCI-H460, and human pancreatic cancer cell line AsPC-1 were cultured in RPMI-1640 containing 10% fetal bovine serum (FBS) (Gibco, Grand Island, NY, USA). Human liver cancer cell line HepG2, human lung cancer cell lines A549 and Calu-1, and human head and neck cancer cell line FaDu were maintained in Dulbecco's Modified Eagle Medium (DMEM) (Gibco) containing 10% FBS. Human ovarian cancer cell line SKOV3 was maintained in McCoy's Medium (Gibco) containing 10% FBS. Natural killer cell lines KHYG-1 and NK-92 were maintained in RPMI-1640 containing 10% FBS and 300 U/ml of interleukin-2 (IL-2). αβ T cells were maintained in RPMI-1640 containing 10% FBS and 500 U/ml of IL-2. The γδ T cells were cultured in RPMI-1640 containing 10% FBS and 1000 U/ml of IL-2.

### (Human αβ T Cell and γδ T Cell Culture)

For αβ T cell culture, peripheral blood mononuclear cells (PBMCs) were placed in a culture dish to which an anti-CD3 antibody (2 μg/ml) and an anti-CD28 antibody (2 μg/ml) were attached and containing RPMI-1640 (10% FBS) containing 300 U/ml of IL-2 and cultured in a cell incubator at 37° C. for seven days. Subculture was performed by replacing the culture medium with a fresh culture medium every two or three days. On day 7, the number of αβ T cells was measured, and the cells were frozen in a freezing solution consisting of 10% dimethyl sulfoxide (DMSO) and 90% FBS.

For γδ T cell culture, PBMCs were cultured in a culture medium containing 5 μM zoledronic acid and 500 U/ml of IL-2 for seven days in a cell incubator at 37° C. Subculture was performed by replacing the culture medium with a fresh culture medium every two or three days. After calculating the number of γδ T cells on day 7, feeder cells irradiated with X-rays at an intensity of 120 Gy were cultured together for seven days at a γδ T cell: feeder cell ratio of 2:1. The number of γδ T cells cultured for 14 days were measured, and the cells were frozen with the same freezing solution as above.

### (Plasmids)

CD19 scFv

A GM-CSF receptor signal sequence, variable light (VL) and heavy chain (VH) regions of an FMC63 anti-CD19 antibody, a hinge and a transmembrane domain of CD28; a hinge and a transmembrane domain of CD8α; a hinge and a transmembrane domain of CD28; an intracellular signaling domain; and intracellular signaling domains of CD28, CD30 and various mutants of CD30, 4-1BB, CD27, ICOS, OX40, and CD3ζ (CD3z) were each artificially synthesized. These were assembled in various combinations using PCR (overlapping extension by PCR). The PCR product amplified by linking XhoI, EcoRV, and NotI sequences to both ends was inserted (fusion cloning, ligation) into the XhoI and NotI sites of a pCI vector (pCI mammalian expression vector, Promega E1731). The PCR product was confirmed by sequencing.

### BCMA

A GM-CSF receptor signal sequence, VL and VH regions of a Bb2121 anti-BCMA antibody, and a hinge and a transmembrane domain of CD28; an intracellular signaling domain; and intracellular signaling domains of CD28, 4-1BB, mutants of CD30, and CD3ζ (CD3z) were each artificially synthesized. As in the CD19 scFv, these were assembled using PCR and inserted into a pCI vector, and the PCR product was confirmed by sequencing.

### EpCAM

An scFv signal sequence (CD8α signal sequence), VL and VH regions of a VB4-845 anti-EpCAM antibody, and a hinge and a transmembrane domain of CD8α; a hinge and a transmembrane domain of CD28; an intracellular signaling domain; and intracellular signaling domains of CD28, 4-1BB, mutants of CD30, and CD3ζ (CD3z) were each artificially synthesized. As in the CD19 scFv, these were assembled using PCR and inserted into a pCI vector, and the PCR product was confirmed by sequencing.

### Mesothelin

An scFv signal sequence (GM-CSF receptor signal sequence), VL and VH regions of a MORAb-009 anti-mesothelin antibody, and a hinge and a transmembrane domain of CD8α; a hinge and a transmembrane domain of CD28; an intracellular signaling domain; and intracellular signaling domains of CD28, 4-1BB, mutants of CD30, and CD3ζ (CD3z) were each artificially synthesized. As in the CD19 scFv, these were assembled using PCR and inserted into a pCI vector, and the PCR product was confirmed by sequencing.

### Glypican-3

An immunoglobulin heavy chain signal sequence, VL and VH regions of a GC33 anti-Glypican-3antibody, and a hinge and a transmembrane domain of CD28; an intracellular signaling domain; and intracellular signaling domains of CD28, 4-1BB, mutants of CD30, and CD3ζ (CD3z) were each artificially synthesized. As in the CD19 scFv, these were assembled using PCR and inserted into a pCI vector, and the PCR product was confirmed by sequencing.

### PD-1 ECD

A signal sequence and extracellular domain of PD-1, a hinge and a transmembrane domain of CD28; an intracellular signaling domain; and intracellular signaling domains of CD28, 4-1BB, mutants of CD30, and CD3ζ (CD3z) were each artificially synthesized. As in the CD19 scFv, these were assembled using PCR and inserted into a pCI vector, and the PCR product was confirmed by sequencing.

### NKp30

A signal sequence and extracellular domain of NKp30, a hinge and a transmembrane domain of CD28; an intracellular signaling domain; and intracellular signaling domains of CD28, 4-1BB, mutants of CD30, and CD3ζ (CD3z) were each artificially synthesized.

As in the CD19 scFv, these were assembled using PCR and inserted into a pCI vector, and the PCR product was confirmed by sequencing.

The above-described CARs are listed in Tables 1 to 13, and their sequences are listed in Table 14. All the CAR domains are linked in tandem with each other and also in frame.

TABLE 1
Second-generation CARs targeting CD19: CD30 domain screening

| | Signal | | | | Sig- | Sig- |
|---|---|---|---|---|---|---|
| Abbreviation | sequence | scFv | Hinge | TM | nal-1 | nal-2 |
| CD19- | GM-CSF | FMC63 | CD28 | CD28 | CD28 | CD3ζ |
| 28S-z | rec | anti- | | | | |
| | | CD19 | | | | |
| CD19- | GM-CSF | FMC63 | CD8α | CD28 | CD30L | CD3ζ |
| 30L-z | rec | anti- | | | | |
| | | CD19 | | | | |
| CD19- | GM-CSF | FMC63 | CD8α | CD28 | CD30M | CD3ζ |
| 30M-z | rec | anti- | | | | |
| | | CD19 | | | | |
| CD19- | GM-CSF | FMC63 | CD8α | CD28 | CD30M^{Mut} | CD3ζ |
| 30M^{Mut}-z | rec | anti- | | | | |
| | | CD19 | | | | |
| CD19- | GM-CSF | FMC63 | CD8α | CD28 | CD30ΔM | CD3ζ |
| 30ΔM-z | rec | anti- | | | | |
| | | CD19 | | | | |
| CD19- | GM-CSF | FMC63 | CD8α | CD28 | CD30S | CD3ζ |
| 30S-z | rec | anti- | | | | |
| | | CD19 | | | | |

CD19-28-z, shown in Table 1, includes a signal sequence domain of the human GM-CSF receptor (1-66 nucleotides (nt), NCBI Reference Sequence ID: 001161531.2); VL and VH regions of an FMC63 anti-CD19 antibody (KR10-2019-7034220/WO2018/200496 JS scFv); a human CD28-derived hinge (340-456 nt, NCBI Reference Sequence: NM_006139.2) and transmembrane domain (457-537 nt, NCBI Reference Sequence: NM_006139.2); a human CD28-derived intracellular signaling domain (538-660 nt, NCBI Reference Sequence: NM_006139.2); and a human CD3z-derived intracellular signaling domain (154-492 nt, 50th_Q deletion, NCBI Reference Sequence: NM_198053.2) linked with a stop codon TAA.

CD19-30L-z is the same as CD19-28-z, except that a human CD8α-derived hinge (412-546 nt, NCBI Reference Sequence: NM_001768) and a human CD30-derived intracellular signaling domain (1219-1785 nt, NCBI Reference Sequence: NM_001243.3) were used.

CD19-30M-z is the same as CD19-28-z, except that a human CD8α-derived hinge (412-546 nt, NCBI Reference Sequence: NM_001768) and a truncated mutant of a human CD30-derived intracellular signaling domain (1501-1785 nt, NCBI Reference Sequence: NM_001243.3) were used.

CD19-30M^{Mut}-z is the same as CD19-28-z, except that a human CD8α-derived hinge (412-546 nt, NCBI Reference Sequence: NM_001768) and a truncated mutant of a human CD30-derived intracellular signaling domain (amino acid 521 of 1501-1785 nt, K→Q, NCBI Reference Sequence: NM_001243.3) were used.

CD19-30ΔM-z is the same as CD19-28-z, except that a human CD8α-derived hinge (412-546 nt, NCBI Reference Sequence: NM_001768) and a truncated mutant of a human CD30-derived intracellular signaling domain (1501-1614 and 1678-1785 nt, NCBI Reference Sequence: NM_001243.3) were used.

CD19-30S-z is the same as CD19-28-z, except that a human CD8α-derived hinge (412-546 nt, NCBI Reference Sequence: NM_001768) and a truncated mutant of a human CD30-derived intracellular signaling domain (1615-1785 nt, NCBI Reference Sequence: NM_001243.3) were used.

TABLE 2
Second-generation CARs targeting CD19: a modified form of CD30S

| | Signal | | | | | | |
|---|---|---|---|---|---|---|---|
| Abbreviation | sequence | scFv | Linker | Hinge | TM | Signal-1 | Signal-2 |
| CD19- | GM-CSF | FMC63 | (G4S)3 | CD28 | CD28 | CD30S | CD3ζ |
| 30S-z | rec | anti- | | | | | |
| | | CD19 | | | | | |
| CD19- | GM-CSF | FMC63 | (G4S)3 | CD28 | CD28 | CD30ΔS | CD3ζ |
| 30ΔS-z | rec | anti- | | | | | |
| | | CD19 | | | | | |
| CD19- | GM-CSF | FMC63 | (G4S)3 | CD28 | CD28 | CD30ΔS^{YN} | CD3ζ |
| 30ΔS^{YN}-z | rec | anti- | | | | | |
| | | CD19 | | | | | |
| CD19- | GM-CSF | FMC63 | (G4S)3 | CD28 | CD28 | CD30ΔS^{YF} | CD3ζ |
| 30ΔS^{YF}-z | rec | anti- | | | | | |
| | | CD19 | | | | | |

CD19-30S-z, shown in Table 2, includes a signal sequence domain of the human GM-CSF receptor (1-66 nt, NCBI Reference Sequence ID: 001161531.2); VL and VH regions of an FMC63 anti-CD19 antibody (KR10-2019-7034220/WO2018/200496 JS scFv); a human CD28-derived hinge (340-456 nt, NCBI Reference Sequence: NM_006139.2) and transmembrane domain (457-537 nt, NCBI Reference Sequence: NM_006139.2); a truncated mutant of a human CD30-derived intracellular signaling domain (1615-1785 nt, NCBI Reference Sequence: NM_001243.3); and a human CD3z-derived intracellular signaling domain (154-492 nt, 50th_Q deletion, NCBI Reference Sequence: NM_198053.2, codon optimization) linked with a stop codon TAA.

CD19-30ΔS-z is the same as CD19-30S-z in Table 2, except that a truncated mutant of a human CD30-derived intracellular signaling domain (1630-1764 nt, NCBI Reference Sequence: NM_001243.3) was used as a first signaling domain.

CD19-30ΔS^{YN}-z is the same as CD19-30S-z in Table 2, except that a domain formed by combining a truncated mutant of a human CD30-derived intracellular signaling domain (1630-1764 nt of CD30) with a YMNM (16 a.a.) region, which is a part of the CD28 intracellular signaling domain, was used as a first signaling domain.

CD19-30ΔS^{YF}-z is the same as CD19-30S-z in Table 2, except that a domain formed by combining a truncated mutant of a human CD30-derived intracellular signaling domain (1630-1764 nt of CD30) with a YMFM (16 a.a.) region, which is a part of the CD28 intracellular signaling domain, was used as a first signaling domain.

TABLE 3
Second-generation CARs targeting CD19

| | Signal | | | | | | |
|---|---|---|---|---|---|---|---|
| Abbreviation | sequence | scFv | Linker | Hinge | TM | Signal-1 | Signal-2 |
| CD19- | GM-CSF | FMC63 | (G4S)3 | CD28 | CD28 | CD30S | CD3ζ |
| 30S-z | rec | anti- | | | | | |
| | | CD19 | | | | | |
| CD19- | GM-CSF | FMC63 | (G4S)3 | CD28 | CD28 | CD28 | CD3ζ |
| 28-z | rec | anti- | | | | | |
| | | CD19 | | | | | |
| CD19-4- | GM-CSF | FMC63 | (G4S)3 | CD28 | CD28 | 4-1BB | CD3ζ |
| 1BB-z | rec | anti- | | | | | |
| | | CD19 | | | | | |
| CD19- | GM-CSF | FMC63 | (G4S)3 | CD28 | CD28 | CD27 | CD3ζ |
| 27-z | rec | anti- | | | | | |
| | | CD19 | | | | | |
| CD19- | GM-CSF | FMC63 | (G4S)3 | CD28 | CD28 | ICOS | CD3ζ |
| ICOS-z | rec | anti- | | | | | |
| | | CD19 | | | | | |
| CD19- | GM-CSF | FMC63 | (G4S)3 | CD28 | CD28 | OX40 | CD3ζ |
| OX40-z | rec | anti- | | | | | |
| | | CD19 | | | | | |

CD19-28-z and CD19-30S-z were produced in the same manner as described above. CD19-4-1BB-z is the same as CD19-30S-z, except that a human CD137 (4-1BB)-derived intracellular signaling domain (640-765 nt, NCBI Reference Sequence: NM_001561.4, codon optimization) was used as a first intracellular signaling domain.

CD19-27-z is the same as CD19-30S-z, except that a human CD27-derived intracellular signaling domain (640-780 nt, NCBI Reference Sequence: NM_001242.4) was used as a first intracellular signaling domain.

CD19-ICOS-z is the same as CD19-30S-z, except that a human ICOS-derived intracellular signaling domain (484-597 nt, NCBI Reference Sequence: NM_012092.2) was used as a first intracellular signaling domain.

CD19-OX40-z is the same as CD19-30S-z, except that a human OX40-derived intracellular signaling domain (706-831 nt, NCBI Reference Sequence: NM_003327.2) was used as a first intracellular signaling domain.

TABLE 4
Third-generation CARs targeting CD19

| | Signal | | | | | | | |
|---|---|---|---|---|---|---|---|---|
| Abbreviation | sequence | scFv | Linker | Hinge | TM | Signal-1 | Signal-2 | Signal-3 |
| CD19-28- | GM- | FMC63 | (G4S)3 | CD28 | CD28 | CD28 | 4-1BB | CD3ζ |
| BB-z | CSF | anti- | | | | | | |
| | rec | CD19 | | | | | | |
| CD19-30S- | GM- | FMC63 | (G4S)3 | CD28 | CD28 | CD30S | 4-1BB | CD3ζ |
| BB-z | CSF | anti- | | | | | | |
| | rec | CD19 | | | | | | |
| CD19-28- | GM- | FMC63 | (G4S)3 | CD28 | CD28 | CD28 | CD30S | CD3ζ |
| 30S-z | CSF | anti- | | | | | | |
| | rec | CD19 | | | | | | |
| CD19-30S- | GM- | FMC63 | (G4S)3 | CD28 | CD28 | CD30S | ICOS | CD3ζ |
| ICOS-z | CSF | anti- | | | | | | |
| | rec | CD19 | | | | | | |
| CD19-30S- | GM- | FMC63 | (G4S)3 | CD28 | CD28 | CD30S | CD27 | CD3ζ |
| CD27-z | CSF | anti- | | | | | | |
| | rec | CD19 | | | | | | |
| CD19-30S- | GM- | FMC63 | (G4S)3 | CD28 | CD28 | CD30S | OX40 | CD3ζ |
| OX40-z | CSF | anti- | | | | | | |
| | rec | CD19 | | | | | | |

CD19-28-BB-z includes a signal sequence domain of the human GM-CSF receptor (1-66 nt, NCBI Reference Sequence ID: 001161531.2); VL and VH regions of an FMC63 anti-CD19 antibody (KR10-2019-7034220/WO2018/200496 JS scFv); a human CD28-derived hinge (340-456 nt, NCBI Reference Sequence: NM_006139.2) and transmembrane domain (457-537 nt, NCBI Reference Sequence: NM_006139.2); a human CD28-derived intracellular signaling domain (538-660 nt, NCBI Reference Sequence: NM_006139.2); and a human CD137 (4-1BB)-derived intracellular signaling domain (640-765 nt, NCBI Reference Sequence: NM_001561.4, codon optimization); and a human CD3z-derived intracellular signaling domain (154-492 nt, 50th_Q deletion, NCBI Reference Sequence: NM_198053.2, codon optimization) linked with a stop codon TAA.

CD19-30S-BB-z is the same as CD19-28-BB-z, except that a truncated mutant of a human CD30-derived intracellular signaling domain (1615-1785 nt, NCBI Reference Sequence: NM_001243.3) was used as a first signaling domain and a human CD137 (4-1BB)-derived intracellular signaling domain (640-765 nt, NCBI Reference Sequence: NM_001561.4, codon optimization) was used as a costimulatory domain (a second signaling domain).

CD19-28-30S-z is the same as CD19-28-BB-z, except that a human CD28-derived hinge (340-456 nt, NCBI Reference Sequence: NM_006139.2) was used as a first signaling domain and a truncated mutant of a human CD30-derived intracellular signaling domain (1615-1785 nt, NCBI Reference Sequence: NM_001243.3) was used as a costimulatory domain (a second signaling domain).

CD19-30S-ICOS-z is the same as CD19-28-BB-z, except that a truncated mutant of a human CD30-derived intracellular signaling domain (1615-1785 nt, NCBI Reference Sequence: NM_001243.3) was used as a first signaling domain and a human ICOS-derived intracellular signaling domain (484-597 nt, NCBI Reference Sequence: NM_012092.2) was used as a costimulatory domain (a second signaling domain).

CD19-30S-CD27-z is the same as CD19-28-BB-z, except that a truncated mutant of a human CD30-derived intracellular signaling domain (1615-1785 nt, NCBI Reference Sequence: NM_001243.3) was used as a first signaling domain and a human CD27-derived intracellular signaling domain (640-780 nt, NCBI Reference Sequence: NM_001242.4) was used as a costimulatory domain (a second signaling domain).

CD19-30S-OX40-z is the same as CD19-28-BB-z, except that a truncated mutant of a human CD30-derived intracellular signaling domain (1615-1785 nt, NCBI Reference Sequence: NM_001243.3) was used as a first signaling domain and a human OX40-derived intracellular signaling domain (706-831 nt, NCBI Reference Sequence: NM_003327.2) was used as a costimulatory domain (a second signaling domain).

TABLE 5
Third-generation CARs targeting BCMA

| | Signal | | | | | | |
|---|---|---|---|---|---|---|---|
| Abbreviation | sequence | scFv | Hinge | TM | Signal-1 | Signal-2 | Signal-3 |
| BCMA-28- | GM-CSF | Anti- | CD28 | CD28 | CD28 | CD30S | CD3ζ |
| 30S-z | rec | BCMA02 | | | | | |
| BCMA- | GM-CSF | Anti- | CD28 | CD28 | CD28 | 4-1BB | CD3ζ |
| 28-BB-z | rec | BCMA02 | | | | | |
| BCMA- | GM-CSF | Anti- | CD28 | CD28 | CD30S | 4-1BB | CD3ζ |
| 30S-BB-z | rec | BCMA02 | | | | | |

BCMA-28-30S-z includes a signal sequence domain of the human GM-CSF receptor (1-66 nt, NCBI Reference Sequence ID: 001161531.2); VL and VH regions of a Bb2121 anti-BCMA antibody (KR10-2021-7003369/WO2020/014333); a human CD28-derived transmembrane domain (457-537 nt, NCBI Reference Sequence: NM_006139.2); a human CD28-derived intracellular signaling domain (538-660 nt, NCBI Reference Sequence: NM_006139.2); a truncated mutant of a human CD30-derived intracellular signaling domain (1615-1785 nt, NCBI Reference Sequence: NM_001243.3); and a human CD3z-derived intracellular signaling domain (154-492 nt, 50th_Q deletion, NCBI Reference Sequence: NM_198053.2) linked with a stop codon TAA.

BCMA-28-BB-z is the same as BCMA-28-30S-z, except that a human CD137 (4-1BB)-derived intracellular signaling domain (640-765 nt, NCBI Reference Sequence: NM_001561.4, codon optimization) was used as a second signaling domain (a costimulatory domain).

BCMA-30S-BB-z is the same as BCMA-28-30S-z, except that a truncated mutant of a human CD30-derived intracellular signaling domain (1615-1785 nt, NCBI Reference Sequence: NM_001243.3) was used as a first signaling domain and a human CD137 (4-1BB)-derived intracellular signaling domain (640-765 nt, NCBI Reference Sequence: NM_001561.4, codon optimization) was used as a second signaling domain (a costimulatory domain).

TABLE 6
Second-generation CARs targeting EpCAM

| Abbre- | Signal | | | | | Sig- | Sig- |
|---|---|---|---|---|---|---|---|
| viation | sequence | scFv | Linker | Hinge | TM | nal-1 | nal-2 |
| EpCAM-2 | CD8α | VB4- | (G4S)3 | CD28 | CD28 | CD28 | CD3ζ |
| 8-z | | 845 | | | | | |
| EpCAM- | CD8α | VB4- | (G4S)3 | CD28 | CD28 | 4-1BB | CD3ζ |
| BB-z | | 845 | | | | | |
| EpCAM- | CD8α | VB4- | (G4S)3 | CD28 | CD28 | CD30S | CD3ζ |
| 30S-z | | 845 | | | | | |
| EpCAM- | CD8α | VB4- | (G4S)3 | CD28 | CD28 | — | — |
| 28TM | | 845 | | | | | |

EpCAM-28-z includes a signal sequence domain of human CD8α (1-63 nt, NCBI Reference Sequence ID: NM_001768.5); a VL region of an optimized VB4-845 anti-EpCAM antibody (PCT/CA2008/001680); and a VH region (PCT/CA2008/001680); a human CD28-derived hinge (340-456 nt, NCBI Reference Sequence: NM_006139.2) and transmembrane domain (457-537 nt, NCBI Reference Sequence: NM_006139.2); a human CD28-derived intracellular signaling domain (538-660 nt, NCBI Reference Sequence: NM_006139.2); and a human CD3z-derived intracellular signaling domain (154-492 nt, 50th_Q deletion, NCBI Reference Sequence: NM_198053.2) linked with a stop codon TAA.

EpCAM-BB-z is the same as EpCAM-28-z, except that a human CD137 (4-1BB)-derived intracellular signaling domain (640-765 nt, NCBI Reference Sequence: NM_001561.4, codon optimization) was used as a first signaling domain.

EpCAM-30S-z is the same as EpCAM-28-z, except that a truncated mutant of a human CD30-derived intracellular signaling domain (1615-1785 nt, NCBI Reference Sequence: NM_001243.3) was used as a first signaling domain.

EpCAM-28TM is the same as EpCAM-28-z, except that it does not include a first signaling domain or a second signaling domain.

TABLE 7
Third-generation CARs targeting EpCAM

| | Signal | | | | | | | |
|---|---|---|---|---|---|---|---|---|
| Abbreviation | sequence | scFv | Linker | Hinge | TM | Signal-1 | Signal-2 | Signal-3 |
| EpCAM- | CD8α | VB4- | (G4S)3 | CD28 | CD28 | CD28 | CD30S | CD3ζ |
| 28-30S-z | | 845 | | | | | | |
| EpCAM- | CD8α | VB4- | (G4S)3 | CD28 | CD28 | CD28 | 4-1BB | CD3ζ |
| 28-BB-z | | 845 | | | | | | |
| EpCAM- | CD8α | VB4- | (G4S)3 | CD28 | CD28 | CD30S | 4-1BB | CD3ζ |
| 30S-BB-z | | 845 | | | | | | |

EpCAM-28-30S-z includes a signal sequence domain of human CD8α (1-63 nt, NCBI Reference Sequence ID: NM_001768.5); a VL region of an optimized VB4-845 anti-EpCAM antibody (PCT/CA2008/001680); and a VH region (PCT/CA2008/001680); a human CD28-derived hinge (340-456 nt, NCBI Reference Sequence: NM_006139.2) and transmembrane domain (457-537 nt, NCBI Reference Sequence: NM_006139.2); a human CD28-derived intracellular signaling domain (538-660 nt, NCBI Reference Sequence: NM_006139.2); a truncated mutant of a human CD30-derived intracellular signaling domain (1615-1785 nt, NCBI Reference Sequence: NM_001243.3); and a human CD3z-derived intracellular signaling domain (154-492 nt, 50th_Q deletion, NCBI Reference Sequence: NM_198053.2) linked with a stop codon TAA.

EpCAM-28-BB-z is the same as EpCAM-28-30S-z, except that a human CD137 (4-1BB)-derived intracellular signaling domain (640-765 nt, NCBI Reference Sequence: NM_001561.4, codon optimization) was used as a second signaling domain (a costimulatory domain).

EpCAM-30S-BB-z is the same as EpCAM-28-30S-z, except that a truncated mutant of a human CD30-derived intracellular signaling domain (1615-1785 nt, NCBI Reference Sequence: NM_001243.3) was used as a first signaling domain and a human CD137 (4-1BB)-derived intracellular signaling domain (640-765 nt, NCBI Reference Sequence: NM_001561.4, codon optimization) was used as a second signaling domain (a costimulatory domain).

TABLE 8
Second-generation CARs targeting MSLN

| | Signal | | | | | | |
|---|---|---|---|---|---|---|---|
| Abbreviation | sequence | scFv | Linker | Hinge | TM | Signal-1 | Signal-2 |
| MSLN(MOR)- | GM-CSF | MORAb- | (G4S)3 | CD28 | CD28 | CD28 | CD3ζ |
| 28-z | rec | 009 | | | | | |
| MSLN(MOR)- | GM-CSF | MORAb- | (G4S)3 | CD28 | CD28 | 4-1BB | CD3ζ |
| BB-z | rec | 009 | | | | | |
| MSLN(MOR)- | GM-CSF | MORAb- | (G4S)3 | CD28 | CD28 | CD30S | CD3ζ |
| 30S-z | rec | 009 | | | | | |

MSLN(MOR)-28-z includes a signal sequence domain of the human GM-CSF receptor (1-66 nt, NCBI Reference Sequence ID: 001161531.2); a VH region (amatuximab, chimeric monoclonal antibody; gamma1 heavy chain, 1-119) and a VL region (amatuximab, chimeric monoclonal antibody; kappa light chain 1-106) of a MORAb-009 anti-mesothelin antibody; a human CD28-derived hinge (340-456 nt, NCBI Reference Sequence: NM_006139.2) and transmembrane domain (457-537 nt, NCBI Reference Sequence: NM_006139.2); a human CD28-derived intracellular signaling domain (538-660 nt, NCBI Reference Sequence: NM_006139.2); and a human CD3z-derived intracellular signaling domain (154-492 nt, 50th_Q deletion, NCBI Reference Sequence: NM_198053.2) linked with a stop codon TAA.

MSLN(MOR)-BB-z is the same as MSLN(MOR)-28-z, except that a human CD137 (4-1BB)-derived intracellular signaling domain (640-765 nt, NCBI Reference Sequence: NM_001561.4, codon optimization) was used as a first signaling domain.

MSLN(MOR)-30S-z is the same as MSLN(MOR)-28-z, except that a truncated mutant of a human CD30-derived intracellular signaling domain (1615-1785 nt, NCBI Reference Sequence: NM_001243.3) was used as a first signaling domain.

TABLE 9
Third-generation CARs targeting MSLN

| | Signal | | | | | | | |
|---|---|---|---|---|---|---|---|---|
| Abbreviation | sequence | scFv | Linker | Hinge | TM | Signal-1 | Signal-2 | Signal-3 |
| MSLN(MOR)- | GM- | MORAb- | (G4S)3 | CD28 | CD28 | CD28 | CD30S | CD3ζ |
| 28-30S-z | CSF rec | 009 | | | | | | |
| MSLN(MOR)- | GM- | MORAb- | (G4S)3 | CD28 | CD28 | CD28 | 4-1BB | CD3ζ |
| 28-BB-z | CSF rec | 009 | | | | | | |
| MSLN(MOR)- | GM- | MORAb- | (G4S)3 | CD28 | CD28 | CD30S | 4-1BB | CD3ζ |
| 30S-BB-z | CSF rec | 009 | | | | | | |

MSLN(MOR)-28-30S-z includes a signal sequence domain of the human GM-CSF receptor (1-66 nt, NCBI Reference Sequence ID: 001161531.2); a VH region (amatuximab, chimeric monoclonal antibody; gamma1 heavy chain, 1-119) and a VL region (amatuximab, chimeric monoclonal antibody; kappa light chain 1-106) of a MORAb-009 anti-mesothelin antibody; a human CD28-derived hinge (340-456 nt, NCBI Reference Sequence: NM_006139.2) and transmembrane domain (457-537 nt, NCBI Reference Sequence: NM_006139.2); a human CD28-derived intracellular signaling domain (538-660 nt, NCBI Reference Sequence: NM_006139.2); a truncated mutant of a human CD30-derived intracellular signaling domain (1615-1785 nt, NCBI Reference Sequence: NM_001243.3); and a human CD3z-derived intracellular signaling domain (154-492 nt, 50th_Q deletion, NCBI Reference Sequence: NM_198053.2, codon optimization) linked with a stop codon TAA.

MSLN(MOR)-28-BB-z is the same as MSLN(MOR)-28-30S-z, except that a human CD137 (4-1BB)-derived intracellular signaling domain (640-765 nt, NCBI Reference Sequence: NM_001561.4, codon optimization) was used as a second signaling domain.

MSLN(MOR)-30S-BB-z is the same as MSLN(MOR)-28-30S-z, except that a truncated mutant of a human CD30-derived intracellular signaling domain (1615-1785 nt, NCBI Reference Sequence: NM_001243.3) was used as a first signaling domain and a human CD137 (4-1BB)-derived intracellular signaling domain (640-765 nt, NCBI Reference Sequence: NM_001561.4, codon optimization) was used as a second signaling domain.

TABLE 10
Third-generation CARs targeting GPC3

| | Signal | | | Sig- | Sig- | Sig- |
|---|---|---|---|---|---|---|
| Abbreviation | sequence | scFv | TM | nal-1 | nal-2 | nal-3 |
| GPC3- | immunoglobulin | GC33 | CD28 | CD28 | CD30S | CD3ζ |
| 28-30S-z | heavy chain | | | | | |
| GPC3- | immunoglobulin | GC33 | CD28 | CD28 | 4-1BB | CD3ζ |
| 28-BB-z | heavy chain | | | | | |
| GPC3- | immunoglobulin | GC33 | CD28 | CD30S | 4-1BB | CD3ζ |
| 30S-BB-z | heavy chain | | | | | |

GPC3-28-30S-z includes a signal sequence domain of a human immunoglobulin heavy chain (1-57 nt, GenBank ID: AAC18316.1); VL and VH regions of a GC33 anti-GPC3 antibody (US2017/0281683); a human CD28-derived transmembrane domain (457-537 nt, NCBI Reference Sequence: NM_006139.2); a human CD28-derived intracellular signaling domain (538-660 nt, NCBI Reference Sequence: NM_006139.2); a truncated mutant of a human CD30-derived intracellular signaling domain (1615-1785 nt, NCBI Reference Sequence: NM_001243.3); and a human CD3z-derived intracellular signaling domain (154-492 nt, 50th_Q deletion, NCBI Reference Sequence: NM_198053.2) linked with a stop codon TAA.

GPC3-28-BB-z is the same as GPC3-28-30S-z, except that a human CD137 (4-1BB)-derived intracellular signaling domain (640-765 nt, NCBI Reference Sequence: NM_001561.4, codon optimization) was used as a second signaling domain.

GPC3-30S-BB-z is the same as GPC3-28-30S-z, except that a truncated mutant of a human CD30-derived intracellular signaling domain (1615-1785 nt, NCBI Reference Sequence: NM_001243.3) was used as a first signaling domain and a human CD137 (4-1BB)-derived intracellular signaling domain (640-765 nt, NCBI Reference Sequence: NM_001561.4, codon optimization) was used as a second signaling domain.

TABLE 11
Second-generation CARs targeting PD-L1

| | Signal | | | | |
|---|---|---|---|---|---|
| Abbreviation | sequence | ECD | TM | Signal-1 | Signal-2 |
| PD-1-28-z | PD-1 | PD-1 | PD-1 5aa + CD28 | CD28 | CD3ζ |
| PD-1-BB-z | PD-1 | PD-1 | PD-1 5aa + CD28 | 4-1BB | CD3ζ |
| PD-1-30S-z | PD-1 | PD-1 | PD-1 5aa + CD28 | CD30S | CD3ζ |
| PD-1-28TM | PD-1 | PD-1 | PD-1 5aa + CD28 | — | — |

PD-1-28-z includes a signal sequence domain of human PD-1 (1-60 nt, NCBI Reference Sequence: NM_005018.2); an extracellular domain of human PD-1 (61-510 nt, NCBI Reference Sequence: NM_005018.2); a transmembrane domain of human PD-1 (511-525 nt, NCBI Reference Sequence: NM_005018.2); a human CD28-derived transmembrane domain (457-537 nt, NCBI Reference Sequence: NM_006139.2); a human CD28-derived intracellular signaling domain (538-660 nt, NCBI Reference Sequence: NM_006139.2); and a human CD3z-derived intracellular signaling domain (154-492 nt, 50th_Q deletion, NCBI Reference Sequence: NM_198053.2) linked with a stop codon TAA.

PD-1-4-BB-z is the same as PD-1-28-z, except that a human CD137 (4-1BB)-derived intracellular signaling domain (640-765 nt, NCBI Reference Sequence: NM_001561.4, codon optimization) was used as a first signaling domain.

PD-1-30S-z is the same as PD-1-28-z, except that a truncated mutant of a human CD30-derived intracellular signaling domain (1615-1785 nt, NCBI Reference Sequence: NM_001243.3) was used as a first signaling domain.

PD-1-28TM is the same as PD-1-28-z, except that it does not include a first signaling domain or a second signaling domain.

TABLE 12
Third-generation CARs targeting PD-L1

| | Signal | | | | | | |
|---|---|---|---|---|---|---|---|
| Abbreviation | sequence | ECD | Hinge | TM | Signal-1 | Signal-2 | Signal-3 |
| PD-1-28- | PD-1 | PD-1 | CD28 | PD-1 5aa + | CD28 | CD30S | CD3ζ |
| 30S-z | | | | CD28 | | | |
| PD-1-28- | PD-1 | PD-1 | CD28 | PD-1 5aa + | CD28 | 4-1BB | CD3ζ |
| BB-z | | | | CD28 | | | |
| PD-1-30S- | PD-1 | PD-1 | CD28 | PD-1 5aa + | CD30S | 4-1BB | CD3ζ |
| BB-z | | | | CD28 | | | |

PD-1-28-30S-z includes a signal sequence domain of human PD-1 (1-60 nt, NCBI Reference Sequence: NM_005018.2); an extracellular domain of human PD-1 (61-510 nt, NCBI Reference Sequence: NM_005018.2); a transmembrane domain of human PD-1 (511-525 nt, NCBI Reference Sequence: NM_005018.2); a human CD28-derived transmembrane domain (457-537 nt, NCBI Reference Sequence: NM_006139.2); a human CD28-derived intracellular signaling domain (538-660 nt, NCBI Reference Sequence: NM_006139.2); a truncated mutant of a human CD30-derived intracellular signaling domain (1615-1785 nt, NCBI Reference Sequence: NM_001243.3); and a human CD3z-derived intracellular signaling domain (154-492 nt, 50th_Q deletion, NCBI Reference Sequence: NM_198053.2, codon optimization) linked with a stop codon TAA.

PD-1-28-BB-z is the same as PD-1-28-30S-z, except that a human CD137 (4-1BB)-derived intracellular signaling domain (640-765 nt, NCBI Reference Sequence: NM_001561.4, codon optimization) was used as a second signaling domain.

PD-1-30S-BB-z is the same as PD-1-28-30S-z, except that a truncated mutant of a human CD30-derived intracellular signaling domain (1615-1785 nt, NCBI Reference Sequence: NM_001243.3) was used as a first signaling domain and a human CD137 (4-1BB)-derived intracellular signaling domain (640-765 nt, NCBI Reference Sequence: NM_001561.4, codon optimization) was used as a second signaling domain.

TABLE 13
Third-generation CARs targeting NKp30

| | Signal | | | | | |
|---|---|---|---|---|---|---|
| Abbreviation | sequence | ECD | TM | Signal-1 | Signal-2 | Signal-3 |
| NKp30-28- | NKp30 | NKp30 | NKp30 | CD28 | CD30S | CD3ζ |
| 30S-z | | | 5aa + CD28 | | | |
| NKp30-28- | NKp30 | NKp30 | NKp30 | CD28 | 4-1BB | CD3ζ |
| BB-z | | | 5aa + CD28 | | | |
| NKp30-30S- | NKp30 | NKp30 | NKp30 | CD30S | 4-1BB | CD3ζ |
| BB-z | | | 5aa + CD28 | | | |

NKp30-28-30S-z includes a signal sequence domain of human NKp30 (1-54 nt, NCBI Reference Sequence ID: NM_147130.1); an extracellular domain of human NKp30 (55-405 nt, NCBI Reference Sequence NM_147130.1); a human NKp30-derived transmembrane domain (406-420 nt, NCBI Reference Sequence: NM_147130.1); a human CD28-derived transmembrane domain (457-537 nt, NCBI Reference Sequence: NM_006139.2); a human CD28-derived intracellular signaling domain (538-660 nt, NCBI Reference Sequence: NM_006139.2); a truncated mutant of a human CD30-derived intracellular signaling domain (1615-1785 nt, NCBI Reference Sequence: NM_001243.3); and a human CD3z-derived intracellular signaling domain (154-492 nt, 50th_Q deletion, NCBI Reference Sequence: NM_198053.2, codon optimization) linked with a stop codon TAA.

NKp30-28-BB-z is the same as NKp30-28-30S-z, except that a human CD137 (4-1BB)-derived intracellular signaling domain (640-765 nt, NCBI Reference Sequence: NM_001561.4, codon optimization) was used as a second signaling domain. NKp30-30S-BB-z is the same as NKp30-28-30S-z, except that a truncated mutant of a human CD30-derived intracellular signaling domain (1615-1785 nt, NCBI Reference Sequence: NM_001243.3) was used as a first signaling domain and a human CD137 (4-1BB)-derived intracellular signaling domain (640-765 nt, NCBI Reference Sequence: NM_001561.4, codon optimization) was used as a second signaling domain.

TABLE 14
Sequence list of CARs described in the examples and domains used in their production

| SEQ | | |
|---|---|---|
| ID | | |
| NO. | Sequence name | Description of sequence |
| 1 | GM-SCF rec | Nucleotide sequence of human GM-CSF receptor signal sequence domain |
| | LS nucleotide | |
| 2 | GM-SCF rec | Amino acid sequence of human GM-CSF receptor signal sequence domain |
| | LS amino acid | |
| 3 | CD19 VL | Nucleotide sequence of CD19 single chain fragment variable-VL |
| | nucleotide | |
| 4 | CD19 VL | Amino acid sequence of CD19 single chain fragment variable-VL |
| | amino acid | |
| 5 | Linker | Nucleotide sequence of the linker connecting CD19 VL and CD19 VH |
| | nucleotide | |
| 6 | Linker | Amino acid sequence of the linker connecting CD19 VL and CD19 VH |
| | amino acid | |
| 7 | CD19 VH | Nucleotide sequence of CD19 single chain fragment variable-VH |
| | nucleotide | |
| 8 | CD19 VH | Amino acid sequence of CD19 single chain fragment variable-VH |
| | amino acid | |
| 9 | Linker | Nucleotide sequence of the linker connecting CD19 scFv and |
| | nucleotide | hinge (gcggccgca) |
| 10 | Linker | Amino acid sequence (AAA) of the linker connecting CD19 scFv and hinge |
| | amino acid | |
| 11 | CD28 Hinge | Nucleotide sequence of CD28-derived spacer domain |
| | nucleotide | |
| 12 | CD28 Hinge | Amino acid sequence of CD28-derived spacer domain |
| | amino acid | |
| 13 | CD28 TM | Nucleotide sequence of CD28-derived transmembrane domain |
| | nucleotide | |
| 14 | CD28 TM | Amino acid sequence of CD28-derived transmembrane domain |
| | amino acid | |
| 15 | CD28 ICD | Nucleotide sequence of CD28-derived intracellular signaling domain |
| | nucleotide | |
| 16 | CD28 ICD | Amino acid sequence of CD28-derived intracellular signaling domain |
| | amino acid | |
| 17 | CD3z | Nucleotide sequence of CD3z-derived intracellular signaling domain |
| | nucleotide | (Δ301-303) |
| 18 | CD3z | Amino acid sequence of CD3z-derived intracellular signaling domain |
| | amino acid | (Δ101) |
| 19 | CD19-28-z | Nucleotide sequence of CD19-28-z CAR (hGMCSF rec LS-CD19 |
| | nucleotide | VL-linker-CD19 VH-AAA-CD28 Hinge-CD28 TM-CD28 ICD-CD3 zeta) |
| 20 | CD19-28-z | Amino acid sequence of CD19-28-z CAR (hGMCSF rec LS-CD19 |
| | amino acid | VL-linker-CD19 VH-AAA-CD28 Hinge-CD28 TM-CD28 ICD-CD3 zeta) |
| 21 | CD8α Hinge | Nucleotide sequence of CD8α-derived spacer domain |
| | nucleotide | |
| 22 | CD8α Hinge | Amino acid sequence of CD8α-derived spacer domain |
| | amino acid | |
| 23 | CD30L ICD | Nucleotide sequence of CD30-derived intracellular signaling domain |
| | nucleotide | (TNFRSF8, 189aa) |
| 24 | CD30L ICD | Amino acid sequence of CD30-derived intracellular signaling domain |
| | amino acid | (TNFRSF8, 189aa) |
| 25 | CD3z | Codon-optimized nucleotide sequence of CD3z-derived intracellular |
| | nucleotide | signaling domain (Δ301-303) |
| 26 | CD3z | Codon-optimized amino acid sequence of CD3z-derived intracellular |
| | amino acid | signaling domain (Δ101) |
| 27 | CD19-30L-z | Nucleotide sequence of CD19-30L-z CAR (hGMCSF rec LS-CD19 |
| | nucleotide | VL-linker-CD19 VH-AAA-CD8α Hinge-CD28 TM-CD30L ICD-CD3 zeta) |
| 28 | CD19-30L-z | Amino acid sequence of CD19-30L-z CAR (hGMCSF rec LS-CD19 |
| | amino acid | VL-linker-CD19 VH-AAA-CD8α Hinge-CD28 TM-CD30L ICD-CD3 zeta) |
| 29 | RSK nucleotide | Nucleotide sequence of three amino acids derived from CD28 (aggagcaag) |
| 30 | RSK amino acid | Sequence of three amino acids derived from CD28 (RSK) |
| 31 | CD30M ICD | Nucleotide sequence of intracellular signaling domain derived |
| | nucleotide | from CD30 (TNFRSF8, 95aa) |
| 32 | CD30M ICD | Amino acid sequence of intracellular signaling domain derived |
| | amino acid | from CD30 (TNFRSF8, 95aa) |
| 33 | CD19-30M-z | Nucleotide sequence of CD19-30M-z CAR (hGMCSF rec LS-CD19 |
| | nucleotide | VL-linker-CD19 VH-AAA-CD8α Hinge-CD28 TM-RSK-CD30M |
| | | ICD-CD3 zeta) |
| 34 | CD19-30M-z | Amino acid sequence of CD19-30M-z CAR (hGMCSF rec LS-CD19 |
| | amino acid | VL-linker-CD19 VH-AAA-CD8α Hinge-CD28 TM-RSK-CD30M |
| | | ICD-CD3 zeta) |
| 35 | CD30M^{mut} ICD | Nucleotide sequence of intracellular signaling domain derived from CD30 |
| | nucleotide | (TNFRSF8, 95aa, 521_KQ) |
| 36 | CD30M^{mut} ICD | Amino acid sequence of intracellular signaling domain derived from CD30 |
| | amino acid | (TNFRSF8, 95aa, 521_KQ) |
| 37 | CD19-30M^{mut}-z | Nucleotide sequence of CD19-30M^{mut}-z CAR (hGMCSF rec LS-CD19 |
| | nucleotide | VL-linker-CD19 VH-AAA-CD8α Hinge-CD28 TM-RSK-30M^{mut} |
| | | ICD-CD3 zeta) |
| 38 | CD19-30M^{mut}-z | Amino acid sequence of CD19-30M^{mut}-z CAR (hGMCSF rec LS-CD19 |
| | amino acid | VL-linker-CD19 VH-AAA-CD8α Hinge-CD28 TM-RSK-30M^{mut} |
| | | ICD-CD3 zeta) |
| 39 | CD30ΔM ICD | Nucleotide sequence of intracellular signaling domain derived from CD30 |
| | nucleotide | (TNFRSF8, 73aa) |
| 40 | CD30ΔM ICD | Amino acid sequence of intracellular signaling domain derived from CD30 |
| | amino acid | (TNFRSF8, 73aa) |
| 41 | CD19-30ΔM-z | Nucleotide sequence of CD19-30ΔM-z CAR (hGMCSF rec LS-CD19 |
| | nucleotide | VL-linker-CD19 VH-AAA-CD8α Hinge-CD28 TM-RSK-ΔCD30M |
| | | ICD-CD3 zeta) |
| 42 | CD19-30ΔM-z | Amino acid sequence of CD19-30ΔM-z CAR (hGMCSF rec LS-CD19 |
| | amino acid | VL-linker-CD19 VH-AAA-CD8α Hinge-CD28 TM-RSK-ΔCD30M |
| | | ICD-CD3 zeta) |
| 43 | CD30S ICD | Nucleotide sequence of CD30-derived intracellular signaling domain |
| | nucleotide | (TNFRSF8, 57aa) |
| 44 | CD30S ICD | Amino acid sequence of CD30-derived intracellular signaling domain |
| | amino acid | (TNFRSF8, 57aa) |
| 45 | CD30ΔS ICD | Nucleotide sequence of intracellular signaling domain derived from CD30 |
| | nucleotide | (TNFRSF8, 45aa) |
| 46 | CD30ΔS ICD | Amino acid sequence of intracellular signaling domain derived from CD30 |
| | amino acid | (TNFRSF8, 45aa) |
| 47 | CD30ΔS^{YN} ICD | Nucleotide sequence of intracellular signaling domains derived from CD30 |
| | nucleotide | (TNFRSF8, 45aa) and CD28 (YMNM, 16aa) |
| 48 | CD30ΔS^{YN} ICD | Amino acid sequence of intracellular signaling domains derived from CD30 |
| | amino acid | (TNFRSF8, 45aa) and CD28 (YMNM, 16aa) |
| 49 | CD30ΔS^{YF} ICD | Nucleotide sequence of intracellular signaling domains derived from CD30 |
| | nucleotide | (TNFRSF8, 45aa) and CD28 (YMNM, 16aa) |
| 50 | CD30ΔS^{YF} ICD | Amino acid sequence of intracellular signaling domains derived from CD30 |
| | amino acid | (TNFRSF8, 45aa) and CD28 (YMNM, 16aa) |
| 51 | CD19-30S-z | Nucleotide sequence of CD19-30S-z CAR (hGMCSF rec LS-CD19 |
| | nucleotide | VL-linker-CD19 VH-AAA-CD8a Hinge-CD28 TM-RSK-CD30S |
| | | ICD-CD3 zeta) |
| 52 | CD19-30S-z | Amino acid sequence of CD19-30S-z CAR (hGMCSF rec LS-CD19 |
| | amino acid | VL-linker-CD19 VH-AAA-CD8a Hinge-CD28 TM-RSK-CD30S |
| | | ICD-CD3 zeta) |
| 53 | (G4S)3 linker | Nucleotide sequence of the linker connecting CD19 VL and CD19 VH |
| | nucleotide | |
| 54 | (G4S)3 linker | Amino acid sequence of the linker connecting CD19 VL and CD19 VH |
| | amino acid | |
| 55 | CD19-30S-z | Nucleotide sequence of CD19-30S-z CAR (hGMCSF rec LS-CD19 |
| | nucleotide | VL-(G4S)3 linker-CD19 VH-AAA-CD28 Hinge-CD28 TM-RSK-CD30S |
| | | ICD-CD3 zeta) |
| 56 | CD19-30S-z | Amino acid sequence of CD19-30S-z CAR (hGMCSF rec LS-CD19 |
| | amino acid | VL-(G4S)3 linker-CD19 VH-AAA-CD28 Hinge-CD28 TM-RSK-CD30S |
| | | ICD-CD3 zeta) |
| 57 | CD19- | Nucleotide sequence of CD19-CD30ΔS-z CAR (hGMCSF rec LS-CD19 |
| | CD30ΔS-z | VL-(G4S)3 linker-CD19 VH-AAA-CD28 Hinge-CD28 TM-RSK-CD30ΔS |
| | nucleotide | ICD-CD3 zeta) |
| 58 | CD19- | Amino acid sequence of CD19-CD30ΔS-z CAR (hGMCSF rec LS-CD19 |
| | CD30ΔS-z | VL-(G4S)3 linker-CD19 VH-AAA-CD28 Hinge-CD28 TM-RSK-CD30ΔS |
| | amino acid | ICD-CD3 zeta) |
| 59 | CD19- | Nucleotide sequence of CD19-CD30ΔS YN-z CAR (hGMCSF rec |
| | CD30ΔS^{YN}-z | LS-CD19 VL-(G4S)3 linker-CD19 VH-AAA-CD28 Hinge-CD28 |
| | nucleotide | TM-RSK-CD30ΔS YN ICD-CD3 zeta) |
| 60 | CD19- | Amino acid sequence of CD19-CD30ΔS YN-z CAR (hGMCSF rec |
| | CD30ΔS^{YN}-z | LS-CD19 VL-(G4S)3 linker-CD19 VH-AAA-CD28 Hinge-CD28 |
| | amino acid | TM-RSK-CD30ΔS YN ICD-CD3 zeta) |
| 61 | CD19- | Nucleotide sequence of CD19-CD30ΔS YF-z CAR (hGMCSF rec |
| | CD30ΔS^{YF}-z | LS-CD19 VL-(G4S)3 linker-CD19 VH-AAA-CD28 Hinge-CD28 |
| | nucleotide | TM-RSK-CD30ΔS YF ICD-CD3 zeta) |
| 62 | CD19- | Amino acid sequence of CD19-CD30ΔS YF-z CAR (hGMCSF rec |
| | CD30ΔS^{YF}-z | LS-CD19 VL-(G4S)3 linker-CD19 VH-AAA-CD28 Hinge-CD28 |
| | amino acid | TM-RSK-CD30ΔS YF ICD-CD3 zeta) |
| 63 | CD19-28-z | Nucleotide sequence of CD19-28-z CAR (hGMCSF rec LS-CD19 VL-(G4S)3 |
| | nucleotide | linker-CD19 VH-AAA-CD28 Hinge-CD28 TM-CD28 ICD-CD3 zeta) |
| 64 | CD19-28-z | Amino acid sequence of CD19-28-z CAR (hGMCSF rec LS-CD19 VL-(G4S)3 |
| | amino acid | linker-CD19 VH-AAA-CD28 Hinge-CD28 TM-CD28 ICD-CD3 zeta) |
| 65 | 4-1BB(CD137) | Codon-optimized nucleotide sequence of intracellular signaling domain |
| | ICD nucleotide | derived from 4-1BB (CD137, TNFRSF9) |
| 66 | 4-1BB(CD137) | Codon-optimized amino acid sequence of intracellular signaling domain |
| | ICD amino acid | derived from 4-1BB (CD137, TNFRSF9) |
| 67 | CD19-BB-z | Nucleotide sequence of CD19-BB-z CAR (hGMCSF rec LS-CD19 VL-(G4S)3 |
| | nucleotide | linker-CD19 VH-AAA-CD28 Hinge-CD28 TM-4-1BB ICD-CD3 zeta) |
| 68 | CD19-BB-z | Amino acid sequence of CD19-BB-z CAR (hGMCSF rec LS-CD19 VL-(G4S)3 |
| | amino acid | linker-CD19 VH-AAA-CD28 Hinge-CD28 TM-4-1BB ICD-CD3 zeta) |
| 69 | CD27 ICD | Nucleotide sequence of CD27-derived intracellular signaling domain |
| | nucleotide | |
| 70 | CD27 ICD | Amino acid sequence of CD27-derived intracellular signaling domain |
| | amino acid | |
| 71 | hCD19-27-z | Nucleotide sequence of CD19-27-z CAR (hGMCSF rec LS-CD19 VL-(G4S)3 |
| | nucleotide | linker-CD19 VH-AAA-CD28 Hinge-CD28 TM-CD27 ICD-CD3 zeta) |
| 72 | hCD19-27-Z | Amino acid sequence of CD19-27-z CAR (hGMCSF rec LS-CD19 VL-(G4S)3 |
| | amino acid | linker-CD19 VH-AAA-CD28 Hinge-CD28 TM-CD27 ICD-CD3 zeta) |
| 73 | ICOS ICD | Nucleotide sequence of ICOS-derived intracellular signaling domain |
| | nucleotide | |
| 74 | ICOS ICD | Amino acid sequence of ICOS-derived intracellular signaling domain |
| | amino acid | |
| 75 | hCD19-ICOS-z | Nucleotide sequence of CD19-ICOS-z CAR (hGMCSF rec LS-CD19 VL-(G4S)3 |
| | nucleotide | linker-CD19 VH-AAA-CD28 Hinge-CD28 TM-ICOS ICD-CD3 zeta) |
| 76 | hCD19-ICOS-z | Amino acid sequence of CD19-ICOS-z CAR (hGMCSF rec LS-CD19 |
| | amino acid | VL-(G4S)3 linker-CD19 VH-AAA-CD28 Hinge-CD28 TM-ICOS ICD-CD3 zeta) |
| 77 | OX40 ICD | Nucleotide sequence of OX40-derived intracellular signaling domain |
| | nucleotide | |
| 78 | OX40 ICD | Amino acid sequence of OX40-derived intracellular signaling domain |
| | amino acid | |
| 79 | hCD19-OX40-z | Nucleotide sequence of CD19-OX40-z CAR (hGMCSF rec LS-CD19 |
| | nucleotide | VL-(G4S)3 linker-CD19 VH-AAA-CD28 Hinge-CD28 TM-OX40 |
| | | ICD-CD3 zeta) |
| 80 | hCD19-OX40-z | Amino acid sequence of CD19-OX40-z CAR (hGMCSF rec |
| | amino acid | LS-CD19 VL-(G4S)3 linker-CD19 VH-AAA-CD28 Hinge-CD28 |
| | | TM-OX40 ICD-CD3 zeta) |
| 81 | hCD19- | Nucleotide sequence of CD19-28-BB-z CAR (hGMCSF rec |
| | 28-BB-z | LS-CD19 VL-(G4S)3 linker-CD19 VH-AAA-CD28 Hinge-CD28 |
| | nucleotide | TM-CD28 ICD-4-1BB ICD-CD3 zeta) |
| 82 | hCD19- | Amino acid sequence of CD19-28-BB-z CAR (hGMCSF rec |
| | 28-BB-z | LS-CD19 VL-(G4S)3 linker-CD19 VH-AAA-CD28 Hinge-CD28 |
| | amino acid | TM-CD28 ICD-4-1BB ICD-CD3 zeta) |
| 83 | hCD19- | Nucleotide sequence of CD19-30S-BB-z CAR (hGMCSF rec |
| | 30S-BB-z | LS-CD19 VL-(G4S)3 linker-CD19 VH-AAA-CD28 Hinge-CD28 |
| | nucleotide | TM-RSK-CD30S ICD-4-1BB ICD-CD3 zeta) |
| 84 | hCD19- | Amino acid sequence of CD19-30S-BB-z CAR (hGMCSF rec |
| | 30S-BB-z | LS-CD19 VL-(G4S)3 linker-CD19 VH-AAA-CD28 Hinge-CD28 |
| | amino acid | TM-RSK-CD30S ICD-4-1BB ICD-CD3 zeta) |
| 85 | hCD19-28- | Nucleotide sequence of CD19-28-30S-z CAR (hGMCSF rec |
| | 30S-z | LS-CD19 VL-(G4S)3 linker-CD19 VH-AAA-CD28 Hinge-CD28 |
| | nucleotide | TM-CD28-CD30S-CD3 zeta) |
| 86 | hCD19-28- | Amino acid sequence of CD19-28-30S-z CAR (hGMCSF rec |
| | 30S-Z | LS-CD19 VL-(G4S)3 linker-CD19 VH-AAA-CD28 Hinge-CD28 |
| | amino acid | TM-CD28-CD30S-CD3 zeta) |
| 87 | hCD19-30S- | Nucleotide sequence of CD19-30S-ICOS-z CAR (hGMCSF |
| | ICOS-Z | rec LS-CD19 VL-(G4S)3 linker-CD19 VH-AAA-CD28 |
| | nucleotide | Hinge-CD28 TM-RSK-CD30S ICD-ICOS ICD-CD3 zeta) |
| 88 | hCD19-30S- | Amino acid sequence of CD19-30S-ICOS-z CAR (hGMCSF |
| | ICOS-Z | rec LS-CD19 VL-(G4S)3 linker-CD19 VH-AAA-CD28 |
| | amino acid | Hinge-CD28 TM-CD30S-RSK-CD27 ICD-CD3 zeta) |
| 89 | hCD19-30S- | Nucleotide sequence of CD19-30S-27-z CAR (hGMCSF rec |
| | 27-Z | LS-CD19 VL-(G4S)3 linker-CD19 VH-AAA-CD28 Hinge-CD28 |
| | nucleotide | TM-RSK-CD30S ICD-CD27 ICD-CD3 zeta) |
| 90 | hCD19-30S- | Amino acid sequence of CD19-30S-27-z CAR (hGMCSF rec |
| | 27-Z | LS-CD19 VL-(G4S)3 linker-CD19 VH-AAA-CD28 Hinge-CD28 |
| | amino acid | TM-RSK-CD30S ICD-CD27 ICD-CD3 zeta) |
| 91 | hCD19-30S- | Nucleotide sequence of CD19-30S-OX40-z CAR (hGMCSF |
| | OX40-Z | rec LS-CD19 VL-(G4S)3 linker-CD19 VH-AAA-CD28 |
| | nucleotide | Hinge-CD28 TM-RSK-CD30S ICD-OX40 ICD-CD3 zeta) |
| 92 | hCD19-30S- | Amino acid sequence of CD19-30S-27-z CAR (hGMCSF rec |
| | OX40-Z | LS-CD19 VL-(G4S)3 linker-CD19 VH-AAA-CD28 Hinge-CD28 |
| | amino acid | TM-RSK-CD30S ICD-OX40 ICD-CD3 zeta) |
| 93 | CD8αLS | Nucleotide sequence of human CD8α signal sequence domain |
| | nucleotide | |
| 94 | CD8α LS | Amino acid sequence of human CD8α signal sequence domain |
| | amino acid | |
| 95 | EpCAM VL | Nucleotide sequence of EpCAM single chain fragment variable-VL |
| | nucleotide | |
| 96 | EpCAM VL | Amino acid sequence of EpCAM single chain fragment variable-VL |
| | amino acid | |
| 97 | EpCAM VH | Nucleotide sequence of EpCAM single chain fragment variable-VH |
| | nucleotide | |
| 98 | EpCAM VH | Amino acid sequence of EpCAM single chain fragment variable-VH |
| | amino acid | |
| 99 | EpCAM-28-z | Nucleotide sequence of EpCAM-28-z CAR (hCD8a LS-EpCAM VL-(G4S)3 |
| | nucleotide | linker-EpCAM VH-CD28 Hinge-CD28 TM-CD28 ICD-CD3 zeta) |
| 100 | EpCAM-28-z | Amino acid sequence of EpCAM-28-z CAR (hCD8a LS-EpCAM VL-(G4S)3 |
| | amino acid | linker-EpCAM VH-CD28 Hinge-CD28 TM-CD28 ICD-CD3 zeta) |
| 101 | EpCAM-BB-z | Nucleotide sequence of EpCAM-BB-z CAR (hCD8a LS-EpCAM VL-(G4S)3 |
| | nucleotide | linker-EpCAM VH-CD28 Hinge-CD28 TM-RSK-4-1BB ICD-CD3 zeta) |
| 102 | EpCAM-BB-z | Amino acid sequence of EpCAM-BB-z CAR (hCD8a LS-EpCAM VL-(G4S)3 |
| | amino acid | linker-EpCAM VH-CD28 Hinge-CD28 TM-RSK-4-1BB ICD-CD3 zeta) |
| 103 | EpCAM-30S-z | Nucleotide sequence of EpCAM-30S-z CAR (hCD8a LS-EpCAM VL-(G4S)3 |
| | nucleotide | linker-EpCAM VH-CD28 Hinge-CD28 TM-RSK-CD30S ICD-CD3 zeta) |
| 104 | EpCAM-30S-z | Amino acid sequence of EpCAM-30S-z CAR (hCD8a LS-EpCAM VL-(G4S)3 |
| | amino acid | linker-EpCAM VH-CD28 Hinge-CD28 TM-RSK-CD30S ICD-CD3 zeta) |
| 105 | EpCAM-28TM | Nucleotide sequence of EpCAM-28TM (hCD8a LS-EpCAM |
| | nucleotide | VL-(G4S)3 linker-EpCAM VH-CD28 Hinge-CD28 TM-RSK-AA) |
| 106 | EpCAM-28TM | Amino acid sequence of EpCAM-28TM (hCD8a LS-EpCAM |
| | amino acid | VL-(G4S)3 linker-EpCAM VH-CD28 Hinge-CD28 TM-RSK-AA) |
| 107 | EpCAM-28- | Nucleotide sequence of EpCAM-28-30S-z CAR (hCD8a LS-EpCAM |
| | 30S-z | VL-(G4S)3 linker-EpCAM VH-CD28 Hinge-CD28 TM-CD28 |
| | nucleotide | ICD-CD30S ICD-CD3 zeta) |
| 108 | EpCAM-28- | Amino acid sequence of EpCAM-28-30S-z CAR (hCD8a LS-EpCAM |
| | 30S-z | VL-(G4S)3 linker-EpCAM VH-CD28 Hinge-CD28 TM-CD28 |
| | amino acid | ICD-CD30S ICD-CD3 zeta) |
| 109 | EpCAM-28- | Nucleotide sequence of EpCAM-28-BB-z CAR (hCD8a LS-EpCAM |
| | BB-z | VL-(G4S)3 linker-EpCAM VH-CD28 Hinge-CD28 TM-CD28 |
| | nucleotide | ICD-4-1BB ICD-CD3 zeta) |
| 110 | EpCAM-28- | Amino acid sequence of EpCAM-28-BB-z CAR (hCD8a LS-EpCAM |
| | BB-z | VL-(G4S)3 linker-EpCAM VH-CD28 Hinge-CD28 TM-CD28 |
| | amino acid | ICD-4-1BB ICD-CD3 zeta) |
| 111 | EpCAM-30S- | Nucleotide sequence of EpCAM-30S-BB-z CAR (hCD8a LS-EpCAM |
| | BB-z | VL-(G4S)3 linker-EpCAM VH-CD28 Hinge-CD28 TM-RSK-CD30S |
| | nucleotide | ICD-4-1BB ICD-CD3 zeta) |
| 112 | EpCAM-30S- | Amino acid sequence of EpCAM-30S-BB-z CAR (hCD8a LS-EpCAM |
| | BB-z | VL-(G4S)3 linker-EpCAM VH-CD28 Hinge-CD28 TM-RSK-CD30S |
| | amino acid | ICD-4-1BB ICD-CD3 zeta) |
| 113 | PD-1 LS | Nucleotide sequence of human PD-1-derived signal sequence |
| | nucleotide | |
| 114 | PD-1 LS | Amino acid sequence of human PD-1-derived signal sequence |
| | amino acid | |
| 115 | PD-1 ECD | Nucleotide sequence of human PD-1-derived extracellular domain |
| | nucleotide | |
| 116 | PD-1 ECD | Amino acid sequence of human PD-1-derived extracellular domain |
| | amino acid | |
| 117 | PD-1 TM | Nucleotide sequence of human PD-1-derived transmembrane domain, 15 bp |
| | nucleotide | |
| 118 | PD-1 TM | Amino acid sequence of human PD-1-derived transmembrane domain, 5aa |
| | amino acid | |
| 119 | PD-1-28-z | Nucleotide sequence of PD-1-28-z CAR (hPD-1 LS-hPD-1 |
| | nucleotide | LS-hPD1TM5aa-CD28 TM-CD28 ICD-CD3 zeta) |
| 120 | PD-1-28-z | Amino acid sequence of PD-1-28-z CAR (hPD-1 LS-hPD-1 |
| | amino acid | LS-hPD1TM5aa-CD28 TM-CD28 ICD-CD3 zeta) |
| 121 | PD-1-BB-z | Nucleotide sequence of PD-1-BB-z CAR (hPD-1 LS-hPD-1 |
| | nucleotide | LS-hPD1TM5aa-CD28 TM-4-1BB ICD-CD3 zeta) |
| 122 | PD-1-BB-z | Amino acid sequence of PD-1-BB-z CAR (hPD-1 LS-hPD-1 |
| | amino acid | LS-hPD1TM5aa-CD28 TM-4-1BB ICD-CD3 zeta) |
| 123 | PD-1-30S-z | Nucleotide sequence of PD-1-30S-z CAR (hPD-1 LS-hPD-1 |
| | nucleotide | LS-hPD1TM5aa-CD28 TM-RSK-CD30S ICD-CD3 zeta) |
| 124 | PD-1-30S-z | Amino acid sequence of PD-1-30S-z CAR (hPD-1 LS-hPD-1 |
| | amino acid | LS-hPD1TM5aa-CD28 TM-RSK-CD30S ICD-CD3 zeta) |
| 125 | PD-1-28TM | Nucleotide sequence of PD-1-28TM (hPD-1 LS-hPD-1 |
| | nucleotide | LS-hPD1TM5aa-CD28 TM-RSK-AA) |
| 126 | PD-1-28TM | Amino acid sequence of PD-1-28TM (hPD-1 LS-hPD-1 |
| | amino acid | LS-hPD1TM5aa-CD28 TM-RSK-AA) |
| 127 | PD-1-28-30S-z | Nucleotide sequence of PD-1-28-30S-z CAR (hPD-1 LS-hPD-1 |
| | nucleotide | LS-hPD1TM5aa-CD28 TM-CD28 ICD-CD30S ICD-CD3 zeta) |
| 128 | PD-1-28-30S-z | Amino acid sequence of PD-1-28-30S-z CAR (hPD-1 LS-hPD-1 |
| | amino acid | LS-hPD1TM5aa-CD28 TM-CD28 ICD-CD30S ICD-CD3 zeta) |
| 129 | PD-1-28-BB-z | Nucleotide sequence of PD-1-28-BB-z CAR (hPD-1 LS-hPD-1 |
| | nucleotide | LS-hPD1TM5aa-CD28 TM-CD28 ICD-4-1BB ICD-CD3 zeta) |
| 130 | PD-1-28-BB-z | Amino acid sequence of PD-1-28-BB-z CAR (hPD-1 LS-hPD-1 |
| | amino acid | LS-hPD1TM5aa-CD28 TM-CD28 ICD-4-1BB ICD-CD3 zeta) |
| 131 | PD-1-30S-BB-z | Nucleotide sequence of PD-1-30S-BB-z CAR (hPD-1 LS-hPD-1 |
| | nucleotide | LS-hPD1TM5aa-CD28 TM-RSK-CD30S ICD-4-1BB ICD-CD3 zeta) |
| 132 | PD-1-30S-BB-z | Amino acid sequence of PD-1-30S-BB-z CAR (hPD-1 LS-hPD-1 |
| | amino acid | LS-hPD1TM5aa-CD28 TM-RSK-CD30S ICD-4-1BB ICD-CD3 zeta) |
| 133 | MSLN (MOR) | Nucleotide sequence of mesothelin single chain variable-VH |
| | VH nucleotide | |
| 134 | MSLN (MOR) | Amino acid sequence of mesothelin single chain fragment variable-VH |
| | VH amino acid | |
| 135 | MSLN (MOR) | Nucleotide sequence of mesothelin single chain fragment variable-VL |
| | VL nucleotide | |
| 136 | MSLN (MOR) | Amino acid sequence of mesothelin single chain fragment variable-VL |
| | VL amino acid | |
| 137 | hMSLN | Nucleotide sequence of hMSLN(MOR)-28-z CAR (hGMCSF |
| | (MOR)-28-z | rec LS-MSLN(MOR) VH-(G4S)3 linker-MSLN(MOR) |
| | nucleotide | VL-CD28hinge-CD28TM-CD28 ICD-CD3 zeta) |
| 138 | hMSLN(MOR)- | Amino acid sequence of hMSLN(MOR)-28-z CAR (hGMCSF |
| | 28-z amino acid | rec LS-MSLN(MOR) VH-(G4S)3 linker-MSLN(MOR) VL-CD28 |
| | | hinge-CD28TM-CD28 ICD-CD3 zeta) |
| 139 | hMSLN(MOR)- | Nucleotide sequence of hMSLN(MOR)-BB-z CAR (hGMCSF |
| | BB-z nucleotide | rec LS-MSLN(MOR) VH-(G4S)3 linker-MSLN(MOR) VL-CD28 |
| | | hinge-CD28TM-4-1BB ICD-CD3 zeta) |
| 140 | hMSLN(MOR)- | Amino acid sequence of hMSLN(MOR)-BB-z CAR (hGMCSF |
| | BB-z | rec LS-MSLN(MOR) VH-(G4S)3 linker-MSLN(MOR) VL-CD28 |
| | amino acid | hinge-CD28TM-4-1BB ICD-CD3 zeta) |
| 141 | hMSLN(MOR)- | Nucleotide sequence of hMSLN(MOR)-30S-z CAR (hGMCSF |
| | 30S-z | rec LS-MSLN(MOR) VH-(G4S)3 linker-MSLN(MOR) VL-CD28 |
| | nucleotide | hinge-CD28TM-RSK-CD30S ICD-CD3 zeta) |
| 142 | hMSLN(MOR)- | Amino acid sequence of hMSLN(MOR)-30S-z CAR (hGMCSF |
| | 30S-z | rec LS-MSLN(MOR) VH-(G4S)3 linker-MSLN(MOR) VL-CD28 |
| | amino acid | hinge-CD28TM-RSK-CD30S ICD-CD3 zeta) |
| 143 | hMSLN(MOR)- | Nucleotide sequence of hMSLN(MOR)-28-30S-z CAR (hGMCSF |
| | 28-30S-z | rec LS-MSLN(MOR) VH-(G4S)3 linker-MSLN(MOR) VL-CD28 |
| | nucleotide | hinge-CD28TM-CD28 ICD-CD30S ICD-CD3 zeta) |
| 144 | hMSLN(MOR)- | Amino acid sequence of hMSLN(MOR)-28-30S-z CAR (hGMCSF |
| | 28-30S-z | rec LS-MSLN(MOR) VH-(G4S)3 linker-MSLN(MOR) |
| | amino acid | VL-CD28hinge-CD28TM-CD28 ICD-CD30S ICD-CD3 zeta) |
| 145 | hMSLN(MOR)- | Nucleotide sequence of hMSLN(MOR)-28-BB-z CAR (hGMCSF |
| | 28-BB-z | rec LS-MSLN(MOR) VH-(G4S)3 linker-MSLN(MOR) VL-CD28 |
| | nucleotide | hinge-CD28TM-CD28 ICD-4-1BB ICD-CD3 zeta) |
| 146 | hMSLN(MOR)- | Amino acid sequence of hMSLN(MOR)-28-BB-z CAR (hGMCSF |
| | 28-BB-z | rec LS-MSLN(MOR) VH-(G4S)3 linker-MSLN(MOR) VL-CD28 |
| | amino acid | hinge-CD28TM-CD28 ICD-4-1BB ICD-CD3 zeta) |
| 147 | hMSLN(MOR)- | Nucleotide sequence of hMSLN(MOR)-30S-BB-z CAR (hGMCSF |
| | 30S-BB-z | rec LS-MSLN(MOR) VH-(G4S)3 linker-MSLN(MOR) VL-CD28 |
| | nucleotide | hinge-CD28TM-RSK-CD30S ICD-4-1BB ICD-CD3 zeta) |
| 148 | hMSLN(MOR)- | Amino acid sequence of hMSLN(MOR)-30S-BB-z CAR (hGMCSF |
| | 30S-BB-z | rec LS-MSLN(MOR) VH-(G4S)3 linker-MSLN(MOR) VL-CD28 |
| | amino acid | hinge-CD28TM-RSK-CD30S ICD-4-1BB ICD-CD3 zeta) |
| 149 | NKp30 LS | Nucleotide sequence of human NKp30-derived signal sequence |
| | nucleotide | (leader sequence) |
| 150 | NKp30 LS | Amino acid sequence of human NKp30-derived signal sequence |
| | amino acid | |
| 151 | NKp30 ECD | Nucleotide sequence of human NKp30-derived extracellular domain |
| | nucleotide | |
| 152 | NKp30 ECD | Amino acid sequence of human NKp30-derived extracellular domain |
| | amino acid | |
| 153 | NKp30 TM | Nucleotide sequence of human NKp30-derived transmembrane domain, 15bp |
| | nucleotide | |
| 154 | NKp30 TM | Amino acid sequence of human NKp30-derived transmembrane domain, 5aa |
| | amino acid | |
| 155 | NKp30-28-30S-z | Nucleotide sequence of NKp30-28-30S-z CAR (hNKp30 LS-hNKp30 |
| | nucleotide | ECD-hNKp30 TM 5aa-CD28TM-CD28 ICD-CD30S ICD-CD3 zeta) |
| 156 | NKp30-28-30S-z | Amino acid sequence of NKp30-28-30S-z CAR (hNKp30 LS-hNKp30 |
| | amino acid | ECD-hNKp30 TM 5aa-CD28TM-CD28 ICD-CD30S ICD-CD3 zeta) |
| 157 | NKp30-28-BB-z | Nucleotide sequence of NKp30-28-BB-z CAR (hNKp30 LS-hNKp30 |
| | nucleotide | ECD-hNKp30 TM 5aa-CD28TM-CD28 ICD-4-1BB ICD-CD3 zeta) |
| 158 | NKp30-28-BB-z | Amino acid sequence of NKp30-28-BB-z CAR (hNKp30 LS-hNKp30 |
| | amino acid | ECD-hNKp30 TM 5aa-CD28TM-CD28 ICD-4-1BB ICD-CD3 zeta) |
| 159 | NKp30-30S-BB-z | Nucleotide sequence of NKp30-30S-BB-z CAR (hNKp30 LS-hNKp30 |
| | nucleotide | ECD-hNKp30 TM 5aa-CD28TM-RSK-CD30S ICD-4-1BB ICD-CD3 zeta) |
| 160 | NKp30-30S-BB-z | Amino acid sequence of NKp30-30S-BB-z CAR (hNKp30 LS-hNKp30 |
| | amino acid | ECD-hNKp30 TM 5aa-CD28TM-RSK-CD30S ICD-4-1BB ICD-CD3 zeta) |
| 161 | Ig LS | Nucleotide sequence of human immunoglobulin heavy chain-derived |
| | nucleotide | signal sequence (leader sequence) |
| 162 | Ig LS | Amino acid sequence of human immunoglobulin heavy chain-derived |
| | amino acid | signal sequence |
| 163 | GPC3 VH | Nucleotide sequence of Glypican3 single chain fragment variable-VH |
| | nucleotide | |
| 164 | GPC3 VH | Amino acid sequence of Glypican3 single chain fragment variable-VH |
| | amino acid | |
| 165 | GPC3 VL | Nucleotide sequence of Glypican3 single chain fragment variable-VL |
| | nucleotide | |
| 166 | GPC3 VL | Amino acid sequence of Glypican3 single chain fragment variable-VL |
| | amino acid | |
| 167 | GPC3-28-30S-z | Nucleotide sequence of GPC3-28-30S-z CAR (immunoglobulin heavy |
| | nucleotide | chain LS-Glypican3 VH-(G4S)3 linker-Glypican3 VL-AAA-CD28 |
| | | hinge-CD28TM-CD28 ICD-CD30S ICD-CD3 zeta) |
| 168 | GPC3-28-30S-z | Amino acid sequence of GPC3-28-30S-z CAR (immunoglobulin heavy |
| | amino acid | chain LS-Glypican3 VH-(G4S)3 linker-Glypican3 VL-AAA-CD28 |
| | | hinge-CD28TM-CD28 ICD-CD30S ICD-CD3 zeta) |
| 169 | GPC3-28-BB-z | Nucleotide sequence of GPC3-28-BB-z CAR (immunoglobulin heavy |
| | nucleotide | chain LS-Glypican3 VH-(G4S)3 linker-Glypican3 VL-AAA-CD28 |
| | | hinge-CD28TM-CD28 ICD-CD30S ICD-CD3 zeta) |
| 170 | GPC3-28-BB-z | Amino acid sequence of GPC3-28-BB-z CAR (immunoglobulin heavy |
| | amino acid | chain LS-Glypican3 VH-(G4S)3 linker-Glypican3 VL-AAA-CD28 |
| | | hinge-CD28TM-CD28 ICD-4-1BB ICD-CD3 zeta) |
| 171 | GPC3-30S-BB-z | Nucleotide sequence of GPC3-30S-BB-z CAR (immunoglobulin heavy |
| | nucleotide | chain LS-Glypican3 VH-(G4S)3 linker-Glypican3 VL-AAA-CD28 |
| | | hinge-CD28TM-CD28 ICD-4-1BB ICD-CD3 zeta) |
| 172 | GPC3-30S-BB-z | Amino acid sequence of GPC3-30S-BB-z CAR (immunoglobulin heavy |
| | amino acid | chain LS-Glypican3 VH-(G4S)3 linker-Glypican3 VL-AAA-CD28 |
| | | hinge-CD28TM-RSK-CD30S ICD-4-1BB ICD-CD3 zeta) |
| 173 | BCMA VH | Nucleotide sequence of BCMA single chain fragment variable-VH |
| | nucleotide | |
| 174 | BCMA VH | Amino acid sequence of BCMA single chain fragment variable-VH |
| | amino acid | |
| 175 | BCMA VL | Nucleotide sequence of BCMA single chain fragment variable-VL |
| | nucleotide | |
| 176 | BCMA VL | Amino acid sequence of BCMA single chain fragment variable-VL |
| | amino acid | |
| 177 | BCMA-28- | Nucleotide sequence of BCMA-28-30S-z CAR (hGMCSF rec |
| | 30S-z | LS-BCMA VH-(G4S)3 linker-BCMA VL-AAA-CD28 Hinge-CD28 |
| | nucleotide | TM-CD28 ICD-CD30S ICD-CD3 zeta) |
| 178 | BCMA-28- | Amino acid sequence of BCMA-28-30S-z CAR (hGMCSF rec |
| | 30S-z | LS-BCMA VH-(G4S)3 linker-BCMA VL-AAA-CD28 Hinge-CD28 |
| | amino acid | TM-CD28 ICD-CD30S ICD-CD3 zeta) |
| 179 | BCMA-28- | Nucleotide sequence of BCMA-28-BB-z CAR (hGMCSF rec |
| | BB-z | LS-BCMA VH-(G4S)3 linker-BCMA VL-AAA-CD28 Hinge-CD28 |
| | nucleotide | TM-CD28 ICD-4-1BB ICD-CD3 zeta) |
| 180 | BCMA-28- | Amino acid sequence of BCMA-28-BB-z CAR (hGMCSF rec |
| | BB-z | LS-BCMA VH-(G4S)3 linker-BCMA VL-AAA-CD28 Hinge-CD28 |
| | amino acid | TM-CD28 ICD-4-1BB ICD-CD3 zeta) |
| 181 | BCMA-30S- | Nucleotide sequence of BCMA-30S-BB-z CAR (hGMCSF rec |
| | BB-z | LS-BCMA VH-(G4S)3 linker-BCMA VL-AAA-CD28 Hinge-CD28 |
| | nucleotide | TM-RSK-CD30S ICD-4-1BB ICD-CD3 zeta) |
| 182 | BCMA-30S- | Amino acid sequence of BCMA-30S-BB-z CAR (hGMCSF rec |
| | BB-z | LS-BCMA VH-(G4S)3 linker-BCMA VL-AAA-CD28 Hinge-CD28 |
| | amino acid | TM-RSK-CD30S ICD-4-1BB ICD-CD3 zeta) |

(mRNA Synthesis)

The above plasmid vectors were amplified using the MN NucleoBond Xtra Midi Plus Endotoxin free kit. The amplified plasmid vectors were linearized using restriction enzymes. The linearized plasmid was used as a template to produce mRNA through in vitro transcription. The MEGAscript® Kit (AM1330, AMBION Inc.) was used to perform the transcription process, followed by DNase treatment, and the final mRNA was produced through 3′polyA tailing using the Poly(A) Tailing kit (AM1350, AMBION). After dispensing the mRNA at 40 μg/tube, the concentration and quality were confirmed using NanoDrop equipment and TapeStation equipment. The mRNA product was stored at −80° C. and used for CAR evaluation.

### (Gene Transfer(Electroporation))

To introduce the CAR gene into KHYG-1 cells and NK-92 cells, 1×10⁷ cells were centrifuged at 1500 rpm for three minutes. After centrifugation, the cells were resuspended in 300 μl of the culture medium opti-MEM. 40 μg of CAR mRNA was added to the cells, and electroporation was performed under the conditions of 200 V, 2 ms, and one time. For αβ T cells and γδ T cells, the CAR gene was introduced into the same number of cells as above under the conditions of 40 μg mRNA at 380 V, 1 ms, and one time.

### (CAR Expression Detection)

To detect anti-CD19 CAR and anti-EpCAM CAR introduced into the NK-92, KHYG-1, αβ T cells, and γδ T cells, the cells were washed using 4% bovine serum albumin (BSA) buffer, and then staining was performed using protein L or human Fab antibodies for 30 minutes. At the end of the reaction time, the cells were washed with 4% BSA buffer and fixed with 2% PFA solution. The group stained with protein L was further stained with a streptavidin-PE antibody for 25 minutes, then washed with 4% BSA buffer and fixed with a 2% PFA solution. For detection of anti-BCMA CAR, anti-MSLN CAR, and anti-GPC3 CAR, staining was performed by the same method as above using a rat Fab antibody. Detection of PD-1 CAR was performed using a human anti-PD-1 antibody. In addition, the NKp30 CAR was detected using used a human anti-NKp30 antibody. The expression ratio in the stained cells was measured using Aurora (Cytek Biosciences) equipment.

### (Luciferase-Based Cytotoxicity)

Target cells into which a luciferase gene was introduced were placed in a 96-well plate at 1×10⁴ cells/well to make 50 ul. 50 ul of effector cells into which the CAR gene was introduced were added at various effector-to-target (E/T) ratios and subjected to a reaction in a cell incubator at 37° C. After reaction for four hours, 100 ul of the Bright-Glo solution was added, and the cells were incubated with shaking at 500 rpm for two minutes. After the incubation, the reaction solution was mixed well with a pipette, 100 μl of the reaction solution was transferred to a white opaque 96-well, and the fluorescence value was measured at each wavelength using a microplate reader.

$$Cytotoxicity was calculated as follows : percent specific lysis = 100 - target with effector / only target \times 100.$$

### (Cytokines)

Target cells were irradiated with 120 Gy X-rays and placed in a 96-well plate at 1×10⁵ cells/well to make 100 ul. Effector cells into which the CAR gene was introduced were also added at 1×10⁵ cells/well to ensure that the E/T ratio was 1. After allowing the cells to react in a cell incubator at 37° C. for 24 hours, the plate was centrifuged at 1500 rpm for five minutes, and 150 μl of the supernatant was collected. The amount of secreted cytokines was measured according to the experimental method of the human CD8/NK multi-analyte flow assay kit (BioLegend, SD, USA).

### <Experimental Example 1> Evaluation of Cytotoxicity of Various Immune Cells Expressing CARs Using Various Types of CD30 Domains as Co-Stimulatory Domain Against CD19 Negative and Positive Blood Cancer Cell Lines

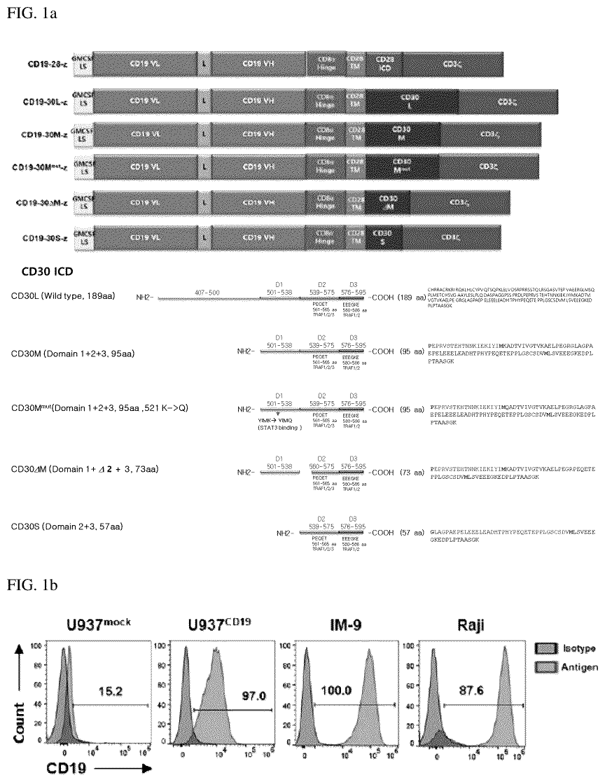

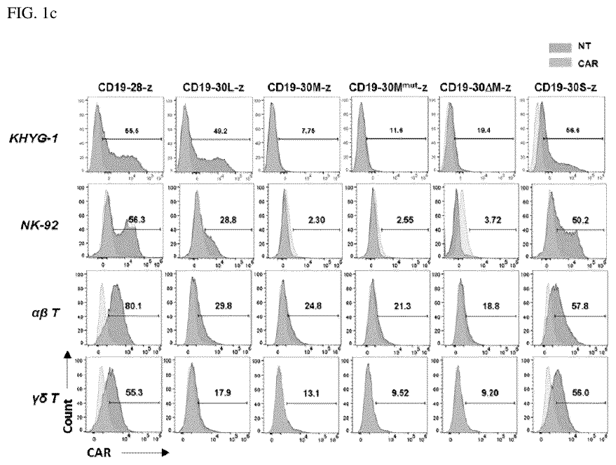

To find the optimal CD30 domain as a costimulatory domain that can increase the anti-tumor efficacy of immune cells, plasmids of the above Example were used with the structures mentioned in Table 1 to produce second-generation CARs targeting a human CD19 antigen and including a CD30 domain (FIG. 1a). The produced second-generation CARs are as follows: CD19-30L-z (1219-1785 nt of the CD30 sequence), CD19-30M-z (1501-1785 nt of the CD30 sequence), CD19-30M^{mut}-z (1501-1785 nt of the CD30 sequence with the amino acid lysine at position 521 changed to glutamine, 521 K→Q), CD19-30ΔM-z (1501-1785 nt of CD30 sequence with deletion of 41615-1677 nt, D1615-1677), and CD19-30S-z (1615-1785 nt of the CD30 sequence).

The CAR mRNA was introduced into natural killer cell lines KHYG-1 and NK-92 as well as t αβ T cells and γδ T cells, and its expression in various immune cells and antitumor efficacy were confirmed.

CD19-28-z, CD19-30L-z, and CD19-30S-z were expressed at high levels in all immune cells, but CD19-30M-z, CD19-30M^{mut}-z, and CD19-30ΔM-z were hardly expressed (FIG. 1c).

In the evaluation of cytotoxic efficacy against CD19 negative and positive blood cancer cell lines, only CD19-28-z and CD19-30S-z specifically killed CD19 positive cancer cell lines (FIG. 1d).

Therefore, the present inventors selected CD30S as the optimal co-stimulatory domain and conducted follow-up experiments.

### <Experimental Example 2> Evaluation of Cytotoxicity of Various Immune Cells Expressing CARs Using Modified Form of the CD30S Domain as Co-Stimulatory Domain Against CD19 Negative and Positive Blood Cancer Cell Lines

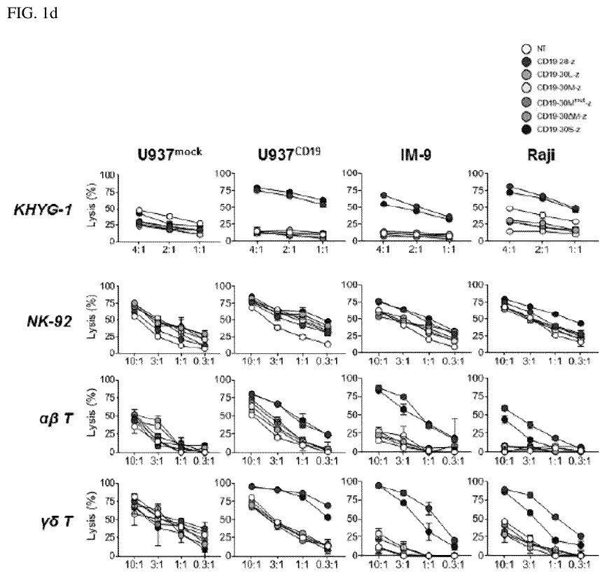

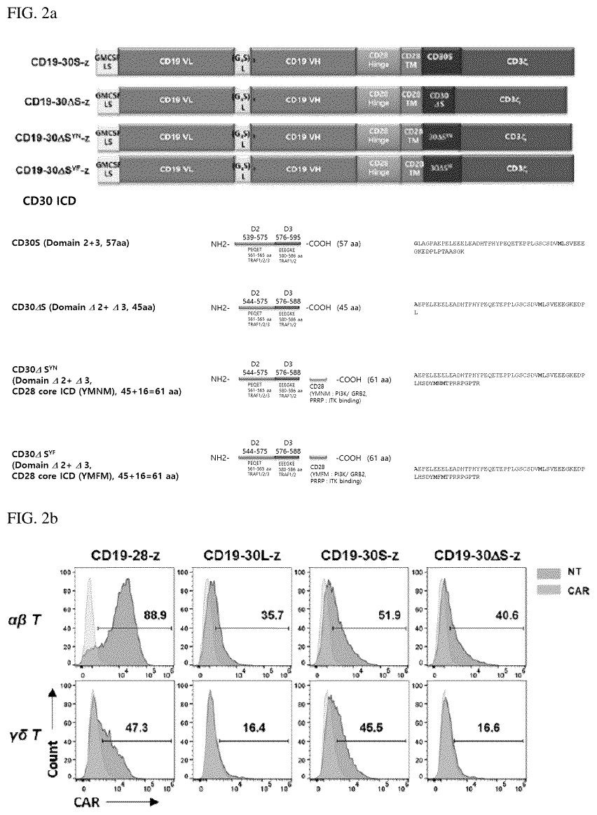

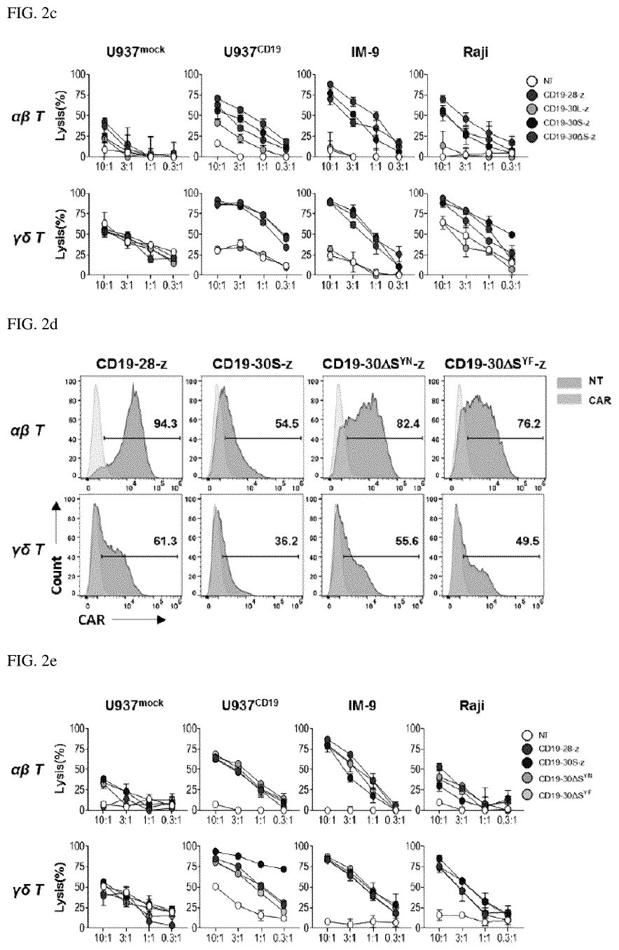

In the above experiment, among the CAR structures including various types of CD30 domains, only the CAR structures including CD30S were stably expressed in various immune cells and exhibited excellent antitumor efficacy. To facilitate future combinations with third-generation CARs using the CD30S domain or with interleukin and chemokine receptors, the present inventors produced a smaller form of the CD30S domain, i.e., CD30ΔS, and evaluated its antitumor efficacy. As shown in FIG. 2a, CD19-28-z, CD19-30L-z, CD19-30S-z, and after CD19-CD30ΔS-z (1630-1764 nt of the CD30 sequence) mRNAs were introduced into αβ T cells and γδ T cells through electroporation, the cytotoxic efficacy against CD19 positive and negative cancer cell lines was evaluated.

CD19-28-z and CD19-30S-z were stably expressed in both the αβ T cells and γδ T cells and exhibited high cytolytic ability. Although the expression of CD19-CD30ΔS-z was relatively low in the γδ T cells compared to the αβ T cells, both types of cells effectively killed CD19 positive blood cancer cells (FIGS. 2b and 2c).

Next, to further improve the function of the CD30ΔS domain, the phosphoinositide 3-kinase binding sites YMNM and YMFM, which are included in the existing costimulatory domains CD28 and ICOS, respectively, were added to CD30ΔS, and antitumor efficacy was evaluated. CD19-28-z, CD19-30S-z, CD19-CD30ΔS^{YN}-z (1630-1764 nt of the CD30 sequence linked with 562-609 nt of the CD28 sequence), and CD19-CD30ΔS^{YF}-z (1630-1764 nt of the CD30 sequence linked with 562-609 nt of the CD28 sequence with the amino acid asparagine at position 193 changed to phenylalanine, N→F) mRNAs were introduced into αβ T cells and γδ T cells.

Both CD19-CD30ΔS^{YN}-z and CD19-CD30ΔS^{YF}-z CARs were stably expressed in the αβ T cells and γδ T cells, and cytotoxic efficacy similar to that of T cells into which existing CD19-28-z and CD19-30S-z CARs were introduced was confirmed (FIGS. 2d and 2e).

### <Experimental Example 3> Comparison of Cytotoxicity and Cytokine Secretion Ability of αβ T Cells and γδ T Cells Expressing CARs Using CD30S or Existing Intracellular Signaling Domain as Co-Stimulatory Domain Against CD19 Negative and Positive Blood Cancer Cell Lines

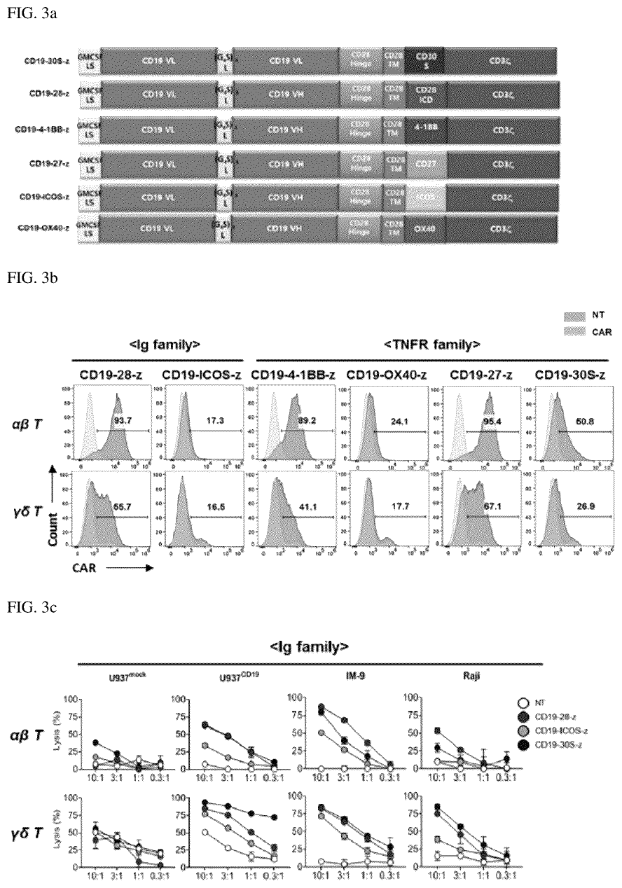

To determine whether αβ T cells and γδ T cells expressing CARs using CD30S or existing ICDs (CD28, 4-1BB, CD27, ICOS, OX40) as a costimulatory domain specifically kill cancer cells, CD19-28-z, CD19-4-1BB-z, CD19-27-z, CD19-ICOS-z, CD19-OX40-z, and CD19-30S-z CAR mRNAs were introduced into αβ T cells and γδ T cells through electroporation (FIG. 3a).

Except for CD19-ICOS-z and CD19-OX40-z, the remaining CARs were stably expressed in the αβ T cells and γδ T cells. Against the CD19 positive cancer cell lines U937CD19, IM-9, and Raji, both the αβ T cells and γδ T cells expressing the CD19-30S-z CAR were confirmed as exhibiting cytotoxic efficacy similar to that of cells expressing CARs including an existing ICD. On the other hand, the CARs exhibited almost no cytolytic activity against the CD19 negative cell line U937^{mock}, similar to the group in which the CAR gene was not introduced (NT) (FIGS. 3b to 3d).

In addition, αβ T cells and γδ T cells expressing the above anti-CD19 second-generation CARs were each cultured together with CD19 positive U937CD19 and negative U937^{mock}, and the amount of the secreted cytokines IFN-γ, TNF-α, and granzyme A, granzyme B, and perforin was measured.

In the αβ T cells, the CD19-30S-z CAR secreted the above-described cytokines in amounts less than those of CD19-28-z but similar to CD19-4-1BB-z. On the other hand, for the CD19 positive cell line, in γδ T cells, the CD19-30S-z CAR secreted IFN-γ and TNF-α in amounts less than those of CD19-28-z and more than those of CD19-4-1BB-Z. CD19-30S-z secreted granzyme A, granzyme B, and perforin in amounts similar to those of CD19-28-z (FIG. 3e).

### <Experimental Example 4> Comparison of Cytotoxicity and Cytokine Secretion Ability of αβ T Cells and γδ T Cells Expressing Third-Generation Type CAR Using CD30S as Co-Stimulatory Domain Against CD19 Negative and Positive Blood Cancer Cell Lines

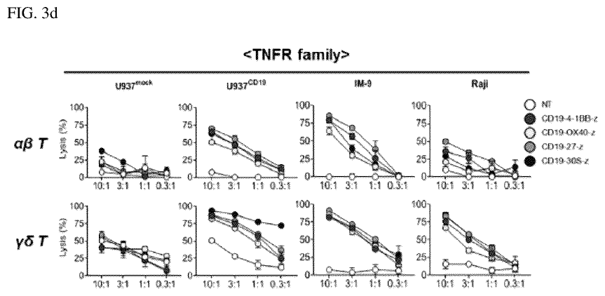

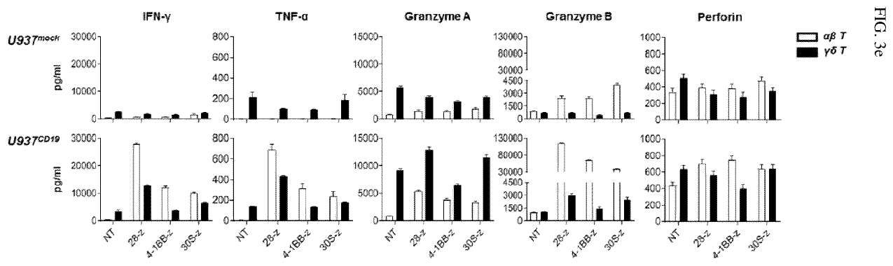

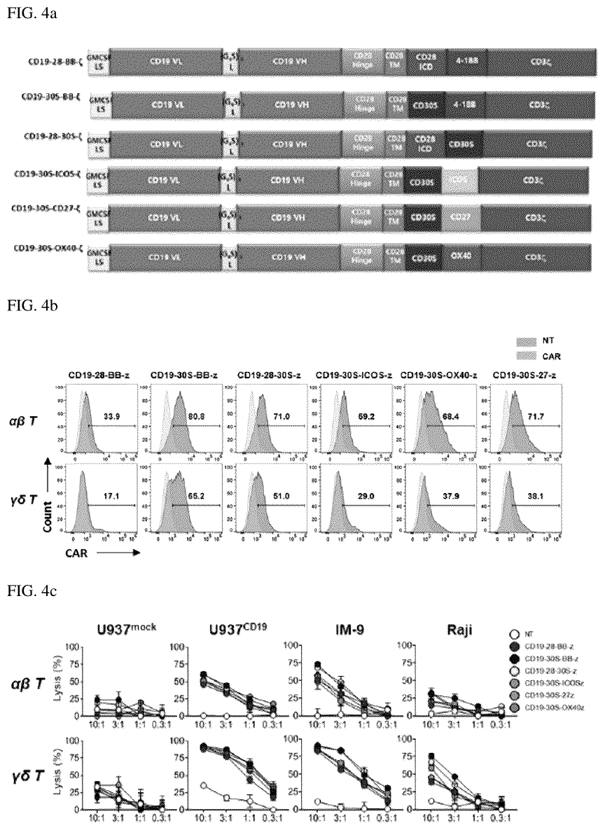

To confirm whether αβ T cells and γδ T cells expressing a third-generation type CAR using CD30S as one costimulatory domain of two costimulatory domains and an existing ICD (CD28, 4-1BB, CD27, ICOS, OX40) as the other react antigen-specifically, plasmid structures mentioned in the above Example were used to produce CD19-28-BB-z, CD19-30S-BB-z, CD19-28-30S-z, CD19-30S-27-z, CD19-30S-ICOS-z, and CD19-30S-OX40-z mRNAs (FIG. 4a).

The anti-CD19 third-generation CAR mRNA was introduced into αβ T cells and γδ T cells using electroporation, and all the CARs were stably expressed in the αβ T cells and γδ T cells (FIG. 4b).

In addition, similar to the existing CD19-28-BB-z, CARs using CD30S as a costimulatory domain (CD19-30S-BB-z, CD19-28-30S-z, CD19-30S-27-z, CD19-30S-ICOS-z, CD19-30S-OX40-z) were confirmed to have specific cytolytic activity against CD19 positive cell lines U937CD19, IM-9, and Raji. On the other hand, they did not exhibit cytolytic activity against the CD19 negative cell line U937^{mock} (FIG. 4c).

In addition, αβ T cells and γδ T cells expressing CD19-28-BB-z, CD19-30S-BB-z, and CD19-28-30S-z CARs were each cultured together with U937^{mock} and U937CD19, and the amount of secreted cytokines was measured.

In both αβ T cells and γδ T cells, CD19-30S-BB-z and CD19-28-30S-z CARs using CD30S as a co-stimulatory domain against CD19 positive U937CD19 secreted IFN-γ, TNF-α, granzyme A, and granzyme B in amounts more than those of CD19-28-BB-z (FIG. 4d).

### <Experimental Example 5> Cytotoxicity Evaluation of γδ T Cells Expressing CAR Targeting Human BCMA Antigen Using CD30S as Co-Stimulatory Domain Against Human Blood Cancer Cell Lines

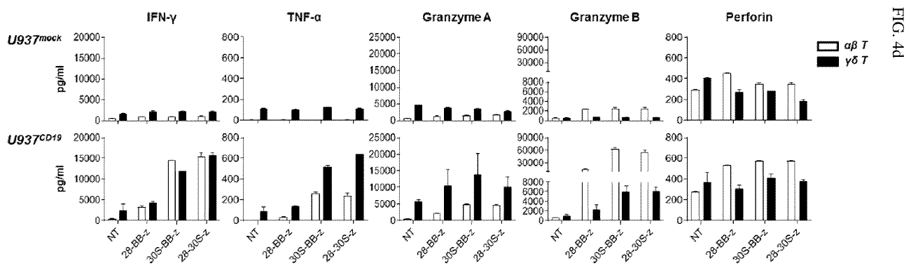

To evaluate the efficacy of the CD30S costimulatory domain in CARs targeting BCMA expressed in human blood cancers, plasmid structures mentioned in the above Example were used to synthesize BCMA-28-BB-z, BCMA-30S-BB-z, and BCMA-28-30S-z mRNAs (FIG. 5a). In addition, human BCMA expression was measured in human blood cancer cell lines U937, IM-9, and Daudi. BCMA was not expressed in U937, but BCMA was expressed in IM-9 and Daudi (FIG. 5b).

BCMA-28-BB-z, BCMA-30S-BB-z, and BCMA-28-30S-z mRNAs were each introduced into γδ T cells using electroporation. BCMA-30S-BB-z and BCMA-28-30S-z, including CD30S, were stably expressed in the γδ T cells, but BCMA-28-BB-z was expressed at a very low level (FIG. 5c). The γδ T cells expressing BCMA-30S-BB-z and BCMA-28-30S-z exhibited high cytolytic activity against BCMA positive cell lines IM-9 and Daudi, but did not exhibit cytolytic activity against the BCMA negative cell line U937 (FIG. 5d).

### <Experimental Example 6> Cytotoxicity Evaluation of αβ T Cells and γδ T Cells Expressing CARs Targeting Human EpCAM Antigen Using CD30S as Co-Stimulatory Domain Against Human Solid Tumor Cell Lines

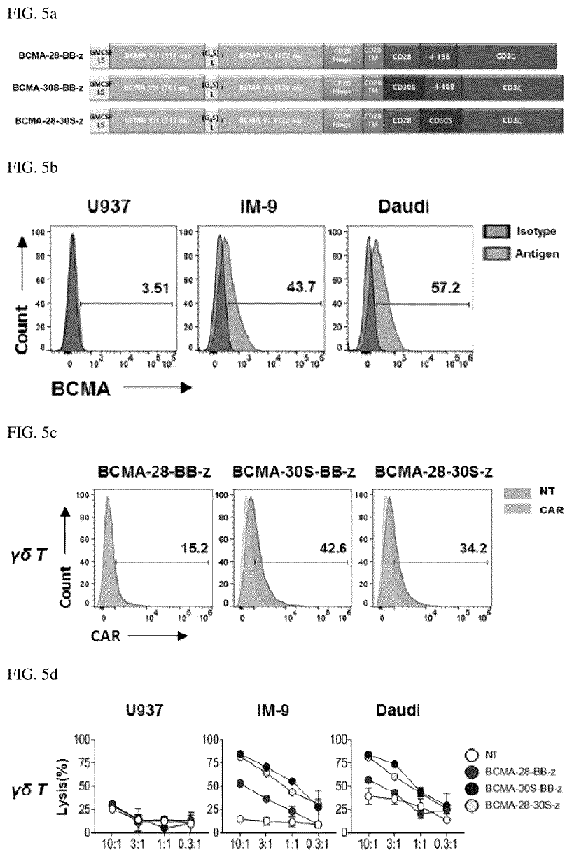

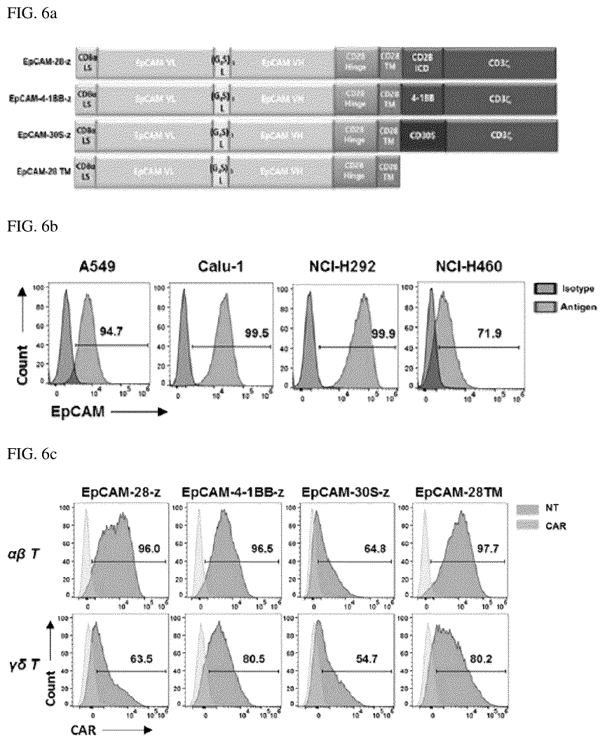

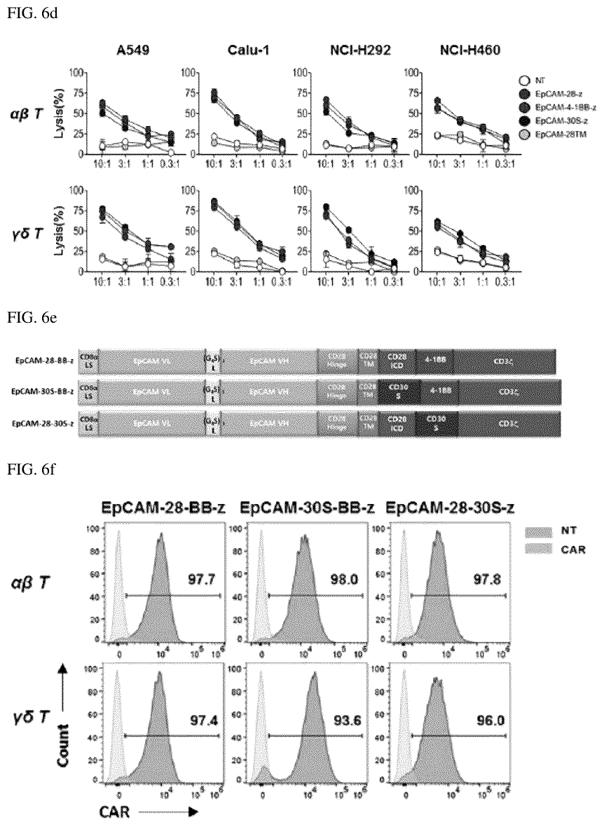

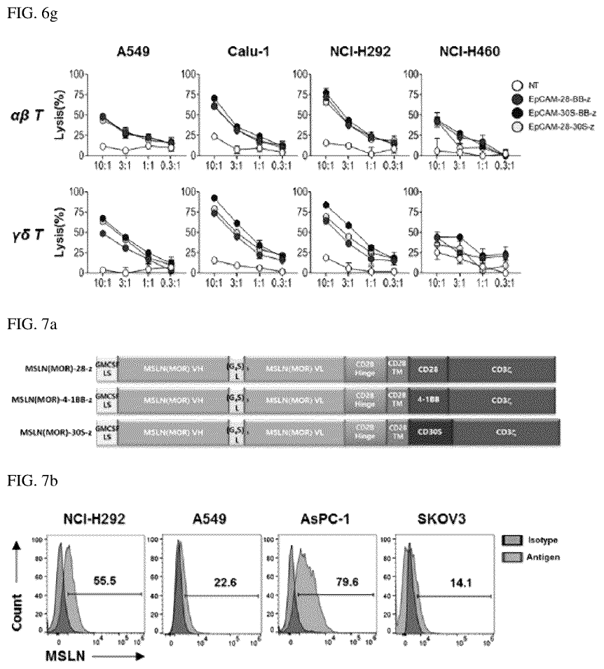

To evaluate the efficacy of the CD30S costimulatory domain in CARs targeting EpCAM expressed in human solid tumors, plasmid structures mentioned in the above Example were used to synthesize EpCAM-28-z, EpCAM-4-1BB-z, EpCAM-30S-z, and EpCAM-28TM mRNAs. In addition, human EpCAM expression was measured in human lung cancer cell lines A549, Calu-1, NCI-H292, and NCI-H460. EpCAM was expressed at 70% or more in lung cancer cell lines A549, Calu-1, NCI-H292, and NCI-H460. EpCAM-28-z, EpCAM-4-1BB-z, EpCAM-30S-z, and EpCAM-28TM mRNAs were each introduced into αβ T cells and γδ T cells using electroporation (FIG. 6a).

EpCAM-28-z, EpCAM-4-1BB-z, EpCAM-30S-z, and EpCAM-28TM CARs were all highly expressed in the αβ T cells and γδ T cells (FIG. 6b). While both the αβ T cells and γδ T cells expressing EpCAM-28-z, EpCAM-4-1BB-z, and EpCAM-30S-z were evaluated as having similar cytolytic activity against lung cancer cell lines expressing EpCAM, the group in which a CAR was not introduced and the group in which EpCAM-28TM was introduced did not exhibit cytolytic activity (FIGS. 6c and 6d).

In addition, to evaluate the efficacy of the CD30S costimulatory domain in the third-generation CAR targeting EpCAM, plasmid structures mentioned in the above Example were used to synthesize EpCAM-28-BB-z, EpCAM-30S-BB-z, and EpCAM-28-30S-z mRNAs, which were each introduced into αβ T cells and γδ T cells (FIG. 6e).

EpCAM-28-BB-z, EpCAM-30S-BB-z, and EpCAM-28-30S-z were all stably expressed in the αβ T cells and γδ T cells (FIG. 6f). The αβ T cells in which the EpCAM third-generation CAR was introduced were evaluated as having excellent cytotoxic efficacy against A549, Calu-1, NCI-H292, and NCI-H460. In the γδ T cells, the EpCAM CARs also effectively killed EpCAM positive lung cancer cell lines (FIG. 6g).

### <Experimental Example 7> Cytotoxicity Evaluation of αβ T Cells and γδ T Cells Expressing CARs Targeting Human Mesothelin Antigen Using CD30S as Co-Stimulatory Domain Against Human Solid Tumor Cell Lines

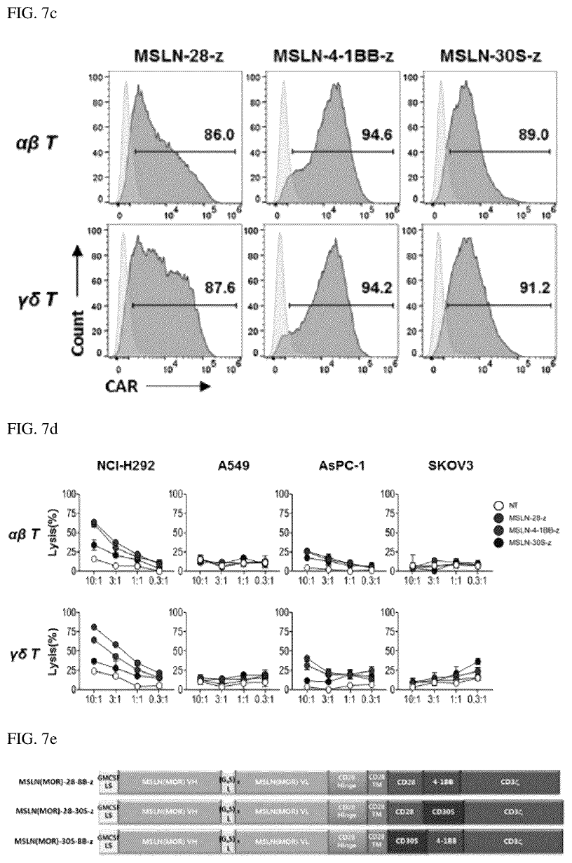

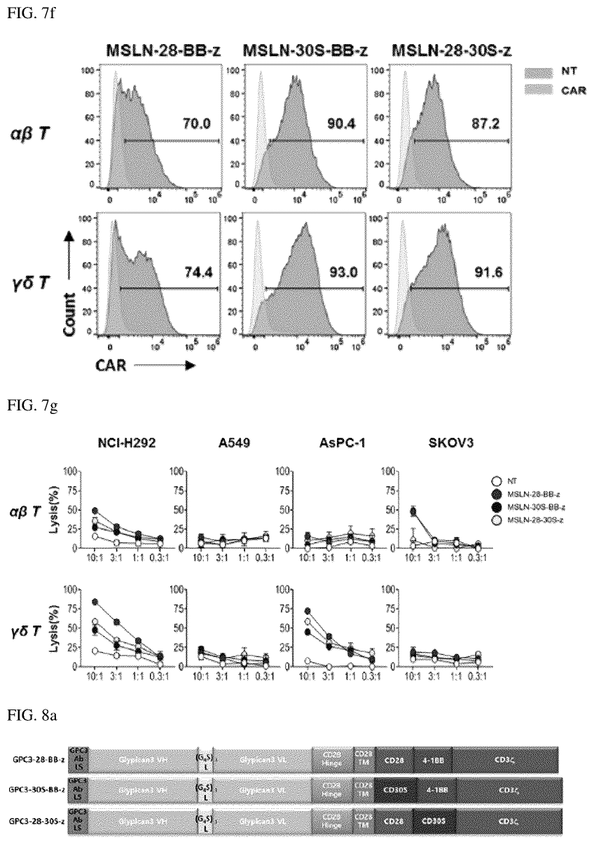

To evaluate the efficacy of the CD30S costimulatory domain in CARs targeting mesothelin expressed in human solid tumors, human mesothelin expression in human lung cancer cell lines A549 and NCI-H292, the human pancreatic cancer cell line AsPC-1, and the human ovarian cancer cell line SKOV3 was measured. Mesothelin was highly expressed in the lung cancer cell line NCI-H292 and the pancreatic cancer cell line AsPC-1, and was expressed at low levels in the lung cancer cell line A549 and ovarian cancer cell line SKOV3 (FIG. 7b). Plasmid structures mentioned in the above Example were used to synthesize MSLN-28-z, MSLN-4-1BB-z, and MSLN-30S-z mRNAs, which were each introduced into αβ T cells and γδ T cells (FIG. 7a).

The above anti-mesothelin second-generation CARs were expressed at high levels in the αβ T cells and γδ T cells (FIG. 7c). The αβ T cells and γδ T cells into which MSLN-28-z and MSLN-4-1BB-z CARs were introduced were confirmed to have cytotoxic efficacy only against mesothelin positive NCI-H292 and AsPC-1. The αβ T cells and γδ T cells into which the MSLN-30S-z CAR was introduced were evaluated to have slightly lower cytotoxic efficacy than those into which MSLN-28-z and MSLN-4-1BB-z CARs were introduced. It was confirmed that MSLN-28-z, MSLN-4-1BB-z, and MSLN-30S-z CARs all exhibited cytotoxic efficacy specifically only against cell lines expressing mesothelin (FIG. 7d).

In addition, to evaluate the efficacy of the CD30S costimulatory domain in the third-generation CAR targeting mesothelin, plasmid structures mentioned in the above Example were used to synthesize MSLN-28-BB-z, MSLN-30S-BB-z, and MSLN-28-30S-z mRNAs, which were each introduced into αβ T cells and γδ T cells using electroporation (FIG. 7e). MSLN-28-BB-z, MSLN-30S-BB-z, and MSLN-28-30S-z CARs were all highly expressed in the αβ T cells and γδ T cells (FIG. 7f). Except for the NT group, the remaining MSLN-CAR αβ T cells exhibited cytolytic activity only against NCI-H292 and did not exhibit cytolytic activity against AsPC-1, SKOV3, and A549. In the γδ T cells, the anti-mesothelin third-generation CARs selectively killed only NCI-H292 and AsPC-1, which highly expressed mesothelin (FIG. 7g).

### <Experimental Example 8> Evaluation of Cytotoxicity of γδ T Cells Expressing CARs Targeting Human GPC3 Antigen Using CD30S as Co-Stimulatory Domain Against Human Solid Tumor Cell Lines

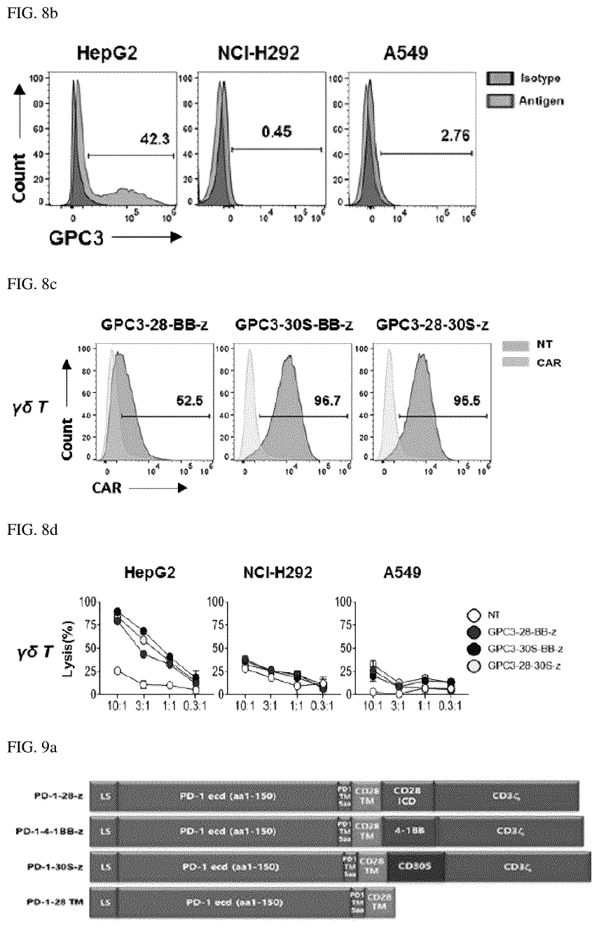

To evaluate the efficacy of the CD30S costimulatory domain in CARs targeting GPC3 expressed in human solid tumors, plasmid structures mentioned in the above Example were used to synthesize GPC3-28-BB-z, GPC3-30S-BB-z, and GPC3-28-30S-z mRNAs (FIG. 8a). Human GPC3 expression was measured in the human liver cancer cell line HepG2 and human lung cancer cell lines NCI-H292 and A549. GPC3 was expressed in the liver cancer cell line HepG2, but not in the lung cancer cell lines NCI-H292 and A549 (FIG. 8b).

GPC3-28-BB-z, GPC3-30S-BB-z, and GPC3-28-30S-z mRNAs were each introduced into αβ T cells and γδ T cells using electroporation. GPC3-28-BB-z, GPC3-30S-BB-z, and GPC3-28-30S-z CARs were all stably expressed in the γδ T cells (FIG. 8c). The γδ T cells expressing GPC3-28-BB-z, GPC3-30S-BB-z, and GPC3-28-30S-z CARs were confirmed to have specific cytolytic activity only against the GPC3 positive cell line HepG2, and they did not exhibit cytolytic activity against the negative cell lines NCI-H292 and A549 (FIG. 8d).

### <Experimental Example 9> Cytotoxicity Evaluation of αβ T Cells and γδ T Cells Expressing CAR Targeting Human PD-L1 Antigen Using CD30S as Co-Stimulatory Domain Against Human Solid Tumor Cell Lines

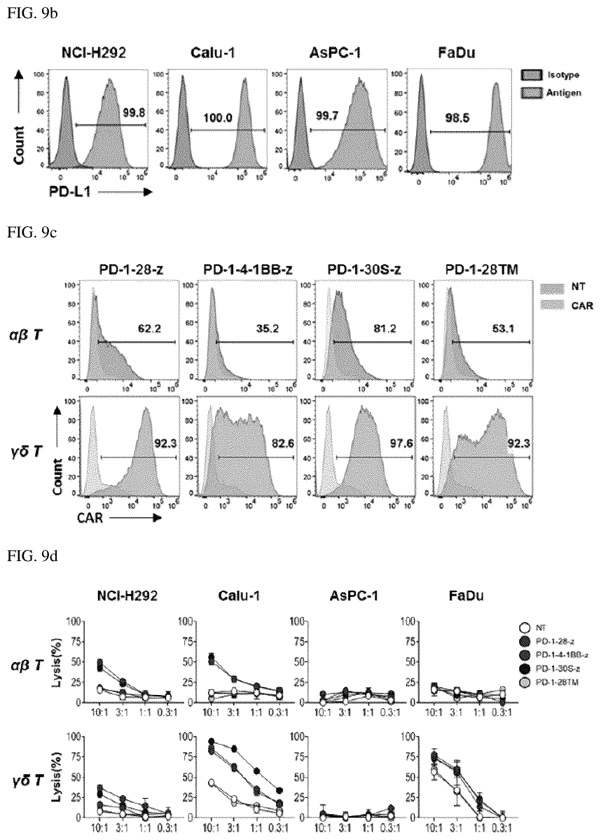

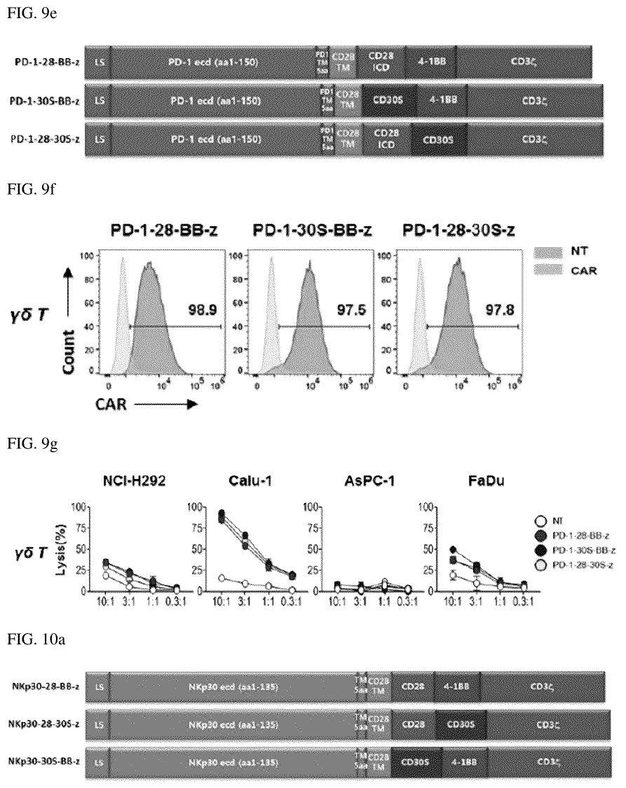

To evaluate the efficacy of the CD30S costimulatory domain in CARs targeting PD-L1 expressed in human solid tumors, plasmid structures mentioned in the above Example were used to synthesize PD-1-28-z, PD-1-4-1BB-z, PD-1-30S-z, and PD-1-28TM mRNAs (FIG. 9a). Human PD-L1 expression was measured in lung cancer cell lines NCI-H292 and Calu-1, the pancreatic cancer cell line AsPC-1, and the head and neck cancer cell line FaDu. PD-L1 was highly expressed in all the cell lines (FIG. 9b). PD-1-28-z, PD-1-4-1BB-z, PD-1-30S-z, and PD-1-28TM mRNAs were each introduced into αβ T cells and γδ T cells using electroporation. PD-1-28-z, PD-1-4-1BB-z, PD-1-30S-z, and PD-1-28TM CARs were all highly expressed in the αβ T cells and γδ T cells (FIG. 9c). In the αβ T cells, CARs excluding NT and PD-1-28TM CAR T cells exhibited cytolytic activity only against NCI-H292 and Calu-1. On the other hand, in the γδ T cells, the above CARs (PD-1-28-z, PD-1-4-1BB-z, PD-1-30S-z) selectively killed NCI-H292, Calu-1, and FaDu. None of the PD-1 CARs exhibited cytolytic activity against the PD-L1 positive cell line AsPC-1 (FIG. 9d).

In addition, to evaluate the efficacy of the CD30S costimulatory domain in third-generation CARs targeting PD-L1, plasmid structures mentioned in the above Example were used to synthesize PD-1-28-BB-z, PD-1-30S-BB-z, and PD-1-28-30S-z mRNAs, which were then introduced into γδ T cells using electroporation (FIG. 9e). PD-1-28-BB-z, PD-1-30S-BB-z, and PD-1-28-30S-z CARs were all highly expressed in the γδ T cells (FIG. 9f). Similar to the cytotoxicity test results of the γδ T cells into which PD-1 second-generation CARs were introduced, the γδ T cells into which PD-1 third-generation CARs were introduced selectively killed NCI-H292, Calu-1, and FaDu, but did not exhibit cytolytic activity against AsPC-1 (FIG. 9g).

### <Experimental Example 10> Cytotoxicity Evaluation of αβ T Cells and γδ T Cells Expressing CAR Targeting Human B7-H6 Antigen Using CD30S as Co-Stimulatory Domain Against Human Solid Tumor Cell Lines

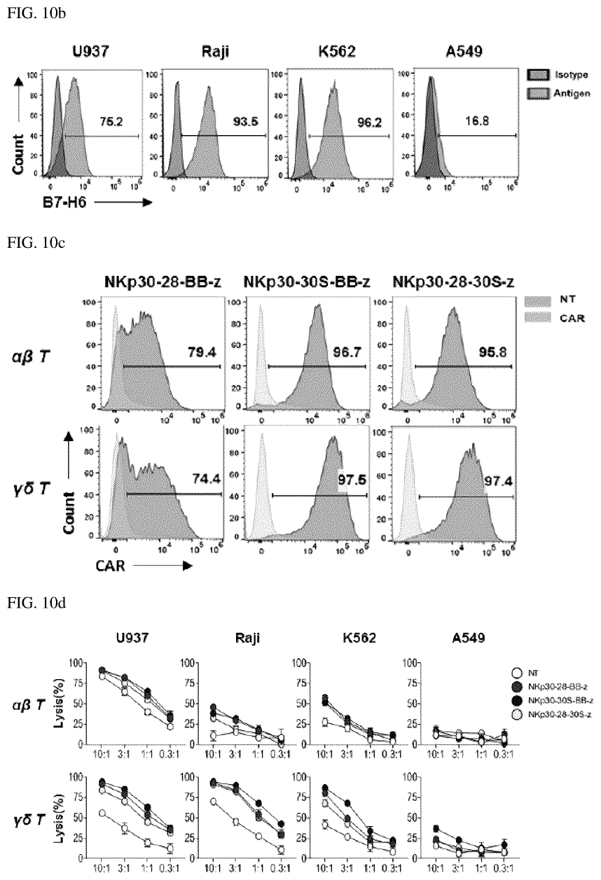

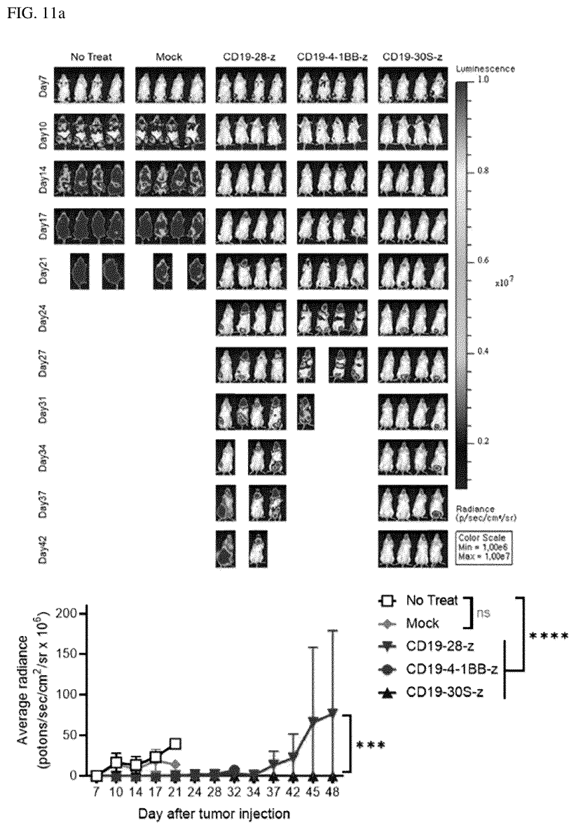

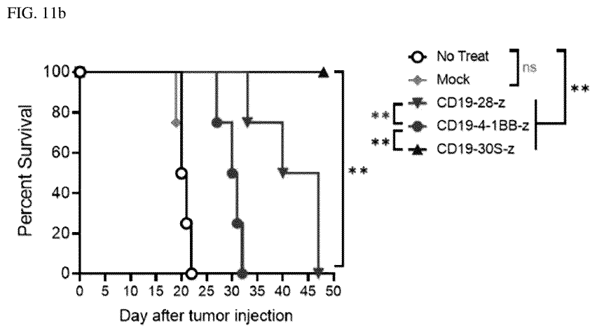

To evaluate the efficacy of the CD30S costimulatory domain in CARs targeting B7-H6 expressed in human solid tumors, plasmid structures mentioned in the above Example were used to synthesize NKp30-28-BB-z, NKp30-30S-BB-z, and NKp30-28-30S-z mRNAs (FIG. 10a). Human B7-H6 expression was measured in human lymphoma cell lines U937 and K562, the human Burkitt's lymphoma cell line Raji, and the human lung cancer cell line A549. In A549, B7-H6 was not expressed, and in U937, Raji, and K562, B7-H6 was expressed at 70% or more (FIG. 10b).

NKp30-28-BB-z, NKp30-30S-BB-z, and NKp30-28-30S-z mRNAs were each introduced into αβ T cells and γδ T cells using electroporation. NKp30-28-BB-z, NKp30-30S-BB-z, and NKp30-28-30S-z CARs were all stably expressed in the αβ T cells and γδ T cells (FIG. 10c). Both the αβ T cells and γδ T cells expressing NKp30-28-BB-z, NKp30-30S-BB-z, and NKp30-28-30S-z CARs were confirmed to have specific cytolytic activity against the B7-H6 positive cell lines U937, Raji, and K562, but they did not exhibit cytolytic activity against the negative cell line A549 (FIG. 10d).

### <Experimental Example 11> Therapeutic Anti-Tumor Effect of T Cells Expressing Second-Generation CD19-CARs According to the Combination of CD30S and Existing ICD (CD28, 4-1BB) in Animal Model Xenografted with Human Leukemia Cell Line NALM6

The anti-tumor activity evaluation and testing methods of second-generation CD19-CAR-expressing T cells are as follows.

### (Mouse, Cell Line)

Six-week-old female NOD/Shi-scid IL2rgamma (null) (NOG, NOD.Cg-Prkdc scid Il2rg tm1sug/JicKoat) mice were purchased from Koatech Inc. (Pyeongtaek, Kyunggi) and bred in an animal laboratory at the Catholic University of Korea under sterile conditions. All procedures of the animal research were carried out in accordance with the guidelines and policies for rodent experiments and the management and use of laboratory animals provided by the Institutional Animal Care and Use Committee (IACUC) of the College of Medicine of the Catholic University of Korea in Seoul (Approval number: CUMS-2022-0285-02) and the Laboratory Animal Welfare Act. NALM6 cells were purchased from the American Type Culture Collection (Manassas, VA), and all cell lines were cultured according to the manufacturer's recommendations.

### (Evaluation of Therapeutic Antitumor Effect)

To evaluate the therapeutic effect in vivo, 1×10⁶ NALM-6-luc-thy1.1 cells were intravenously administered to the mouse tail and then on day 7, 1×10⁷ CD19-CAR-αβ T cells were intravenously administered once. Mice that did not receive cell treatment (No Treat) were included as a control group. Tumor growth was monitored over time by in vivo bioluminescence imaging in individual mice, and animal body weight was measured at fixed times. Mice that lost 20% or more body weight were euthanized according to an approved laboratory animal protocol.

Tumor growth was monitored over time by detecting bioluminescence in whole animals using the in vivo imaging system (IVIS) Lumina x-ray multi-species optical imaging system (XRMS) (in vivo luminescence bioimaging). Luciferin (Promega, P1043) was prepared at 15 mg/mL using a sterile saline solution, stored in a freezer at −80° C., and thawed at 37° C. before use. To confirm bioluminescence, the mice received an intraperitoneal injection of 3 mg/200 ul of luciferin and allowed to react for a reaction time of seven to eight minutes. After the reaction time, the mice were maintained under anesthesia throughout the entire imaging process through a nose cone isoflurane oxygen delivery device, while bioluminescence was measured in a light-tight chamber. In tumor growth image analysis, the evaluation was performed by quantifying the average luminescence of in vivo bioluminescence in individual mice.

As shown in FIGS. 11a and 11b, an animal model xenografted with the human leukemia cell line NALM6 was established to evaluate the in vivo efficacy of the αβ T cell therapy transduced with CD30S targeting CD19 or a second-generation CAR used as an existing co-stimulatory domain. As a result, the anti-tumor effect of CD19-28-z, CD19-4-1BB-z, and CD19-30S-z CAR-αβ T cells exhibited the therapeutic benefit of delaying tumor growth and increasing survival ability compared to the non-transduced αβ T cell therapy. Among them, the CD19-30S-z CAR-αβ T cell treatment group had a substantially higher antitumor effect than the CD19-28-z, CD19-4-1BB-z, and CAR-αβ T cell treatment groups, and no significant toxicity was observed.

The present invention can be used as an immune cell therapy in the field of tumor treatment.

1. A chimeric antigen receptor comprising:
a target antigen binding domain;
a transmembrane domain;
a CD30-derived intracellular signaling domain selected from an amino acid sequence of SEQ ID NOs: 44, 46, 48 or 50; and
a CD3ζ intracellular signaling domain.

2. The chimeric antigen receptor of claim 1, wherein the target antigen binding domain includes an antibody or a fragment thereof that specifically binds to the target antigen.

3. The chimeric antigen receptor of claim 1, wherein the target antigen is selected from the group consisting of CD19, MUC16, MUC1, CAIX, CEA, CDS, CD7, CD10, CD20, CD22, CD30, CD33, CD34, CD38, CD41, CD44, CD49f, CD56, CD74, CD133, CD138, BCMA, PD-1, NKp30, cytomegalovirus (CMV) infected cell antigen, EGP-2, EGP-40, EpCAM, Erb-B2, Erb-B3, Erb-B4, FBP, fetal acetylcholine receptor, folate receptor-α, GD2, GD3, HER-2, hTERT, IL-13R-a2, K-light chain, KDR, LeY, L1 cell adhesion molecule, MAGE-A1, mesothelin, NKG2D ligand, NY-ESO-1, carcinoembryonic antigen (h5T4), PSCA, PSMA, ROR1, TAG-72, VEGF-R2, WT-1, CD24, CD47, EGFR, CD123, GPC3, CD117, Claudin18.2, cMET, EphA2, CLL-1, CD171, AXL, ROR2, FAP, and CD5.

4. The chimeric antigen receptor of claim 1, wherein the transmembrane domain is a transmembrane domain of a TCR co-receptor or T cell costimulatory molecule selected from the group consisting of CD8, 4-1BB, CD27, CD28, CD30, 0X40, CD3e, CD3ζ, CD45, CD4, CD5, CD9, CD16, CD22, CD33, CD37, CD64, CD80, CD86, CD134, CD137, CD154, and ICOS (CD278), or derived therefrom.

5. The chimeric antigen receptor of claim 1, further comprising a hinge region between a C-terminus of the target antigen binding domain and an N-terminus of the transmembrane domain.

6. The chimeric antigen receptor of claim 1, further comprising an intracellular signaling domain selected from the group consisting of CD27, CD28, 4-1BB (CD137), 0X40, CD40, ICOS, lymphocyte function-associated antigen-1 (LFA-1), CD2, CD7, NKG2C, CD83, Dap10, GITR, OX40L, Myd88-CD40, and KIR2DS2.

7. The chimeric antigen receptor of claim 1, further comprising a signal peptide at an N-terminus of the target antigen binding domain, wherein the signal peptide is selected from the group consisting of a GM-CSF receptor signal sequence, a CD8α signal sequence, an immunoglobulin heavy chain signal sequence, a PD-1 signal sequence, and an NKp30 signal sequence.

8. A nucleic acid molecule encoding the chimeric antigen receptor of claim 1.

9. A vector comprising the nucleic acid molecule of claim 8.

10. An isolated immune effector cell comprising the chimeric antigen receptor of claim 1, the nucleic acid molecule of claim 8, or the vector of claim 9.

11. The immune effector cell of claim 10, wherein the immune effector cell is an αβ T cell, a γδ T cell, an NK cell, an NK T cell, or a macrophage.

12. An anti-tumor composition comprising the immune effector cell of claim 10.

13. A method of treating cancer, comprising administering a therapeutically effective amount of the immune effector cell of claim 10 to a subject in need thereof.

14. The method of treating cancer of claim 13, wherein the cancer is selected from lung cancer, colorectal cancer, prostate cancer, thyroid cancer, breast cancer, brain cancer, head and neck cancer, esophageal cancer, skin cancer, melanoma, retinoblastoma, thymus cancer, stomach cancer, colon cancer, liver cancer, ovarian cancer, uterine cancer, bladder cancer, rectal cancer, gallbladder cancer, biliary tract cancer, pancreatic cancer, lymphoma, leukemia, or multiple myeloma.


SEQUENCE LISTING (182 sequences)

SEQ ID NO: 1 - DNA, 71 bp, synthetic construct
  source 1..71: mol_type = other DNA
atgcttctcctggtgacaagccttctgctctgtgagttaccacacccagcattcctcctgatcccagatat

SEQ ID NO: 2 - protein, 22 aa, synthetic construct
MLLLVTSLLLCELPHPAFLLIP

SEQ ID NO: 3 - DNA, 312 bp, synthetic construct
  source 1..312: mol_type = other DNA
atgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaa

SEQ ID NO: 4 - protein, 107 aa, synthetic construct
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 5 - DNA, 54 bp, synthetic construct
  source 1..54: mol_type = other DNA
ggctcgacgagcggctctggtaaaccgggctctggtgaaggcagtaccaaaggt

SEQ ID NO: 6 - protein, 18 aa, synthetic construct
GSTSGSGKPGSGEGSTKG

SEQ ID NO: 7 - DNA, 360 bp, synthetic construct
  source 1..360: mol_type = other DNA
gaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtca

SEQ ID NO: 8 - protein, 120 aa, synthetic construct
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 9-10 - skipped (2 entries)

SEQ ID NO: 11 - DNA, 117 bp, synthetic construct
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 12 - protein, 39 aa, synthetic construct
IEVMYPPPYLDNEKSNGTIIHVKGKHLCPSPLFPGPSKP

SEQ ID NO: 13 - DNA, 81 bp, synthetic construct
  source 1..81: mol_type = other DNA
ttttgggtgctggtggtggttgggggagtcctggcttgctatagcttgctagtaacagtggcctttattattttctgggtg

SEQ ID NO: 14 - protein, 27 aa, synthetic construct
FWVLVVVGGVLACYSLLVTVAFIIFWV

SEQ ID NO: 15 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 16 - protein, 41 aa, synthetic construct
RSKRSRLLHSDYMNMTPRRPGPTRKHYQPYAPPRDFAAYRS

SEQ ID NO: 17 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 18 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 1470 bp, synthetic construct
  source 1..1470: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggctcgacgagcggctctggtaaaccgggctctggtgaaggcagtaccaaaggtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 20 - protein, 489 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGSTSGSGKPGSGEGSTKGEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 135 bp, synthetic construct
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 22 - protein, 45 aa, synthetic construct
TTTPAPRPPTPAPTIASQPLSLRPEACRPAAGGAVHTRGLDFACD

SEQ ID NO: 23 - DNA, 567 bp, synthetic construct
  source 1..567: mol_type = other DNA
tgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 24 - protein, 189 aa, synthetic construct
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 25 - DNA, 339 bp, synthetic construct
  source 1..339: mol_type = other DNA
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 26 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1932 bp, synthetic construct
  source 1..1932: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggctcgacgagcggctctggtaaaccgggctctggtgaaggcagtaccaaaggtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 28 - protein, 643 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGSTSGSGKPGSGEGSTKGEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAATTTPAPRPPTPAPTIASQPLSLRPEACRPAAGGAVHTRGLDFACDFWVLVVVGGVLACYSLLVTVAFIIFWVCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29-30 - skipped (2 entries)

SEQ ID NO: 31 - DNA, 285 bp, synthetic construct
  source 1..285: mol_type = other DNA
cctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 32 - protein, 95 aa, synthetic construct
PEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 33 - DNA, 1659 bp, synthetic construct
  source 1..1659: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggctcgacgagcggctctggtaaaccgggctctggtgaaggcagtaccaaaggtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 34 - protein, 552 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGSTSGSGKPGSGEGSTKGEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAATTTPAPRPPTPAPTIASQPLSLRPEACRPAAGGAVHTRGLDFACDFWVLVVVGGVLACYSLLVTVAFIIFWVRSKPEPRVSTEHTNNKIEKIYIMKADTVIVGTVKAELPEGRGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 285 bp, synthetic construct
  source 1..285: mol_type = other DNA
cctgagccccgggtgtccacggagcacaccaataacaagattgagaaaatctacatcatgcaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 36 - protein, 95 aa, synthetic construct
PEPRVSTEHTNNKIEKIYIMQADTVIVGTVKAELPEGRGLAGPAEPELEEELEADHTPHYPEQETEPPLGSCSDVMLSVEEEGKEDPLPTAASGK

SEQ ID NO: 37 - DNA, 1659 bp, synthetic construct
  source 1..1659: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggctcgacgagcggctctggtaaaccgggctctggtgaaggcagtaccaaaggtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcctgagccccgggtgtccacggagcacaccaataacaagattgagaaaatctacatcatgcaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 38 - protein, 552 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGSTSGSGKPGSGEGSTKGEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAATTTPAPRPPTPAPTIASQPLSLRPEACRPAAGGAVHTRGLDFACDFWVLVVVGGVLACYSLLVTVAFIIFWVRSKPEPRVSTEHTNNKIEKIYIMQADTVIVGTVKAELPEGRGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 219 bp, synthetic construct
  source 1..219: mol_type = other DNA
cctgagccccgggtgtccacggagcacaccaataacaagattgagaaaatctacatcatgaaggctgacaccgtgatcgtggggaccgtgaaggctgagctgccggagggccggcccgagcaggagacagaaccgcctctgggcagctgcagcgatgtcatgctctcagtggaagaggaagggaaagaagaccccttgcccacagctgcctctggaaag

SEQ ID NO: 40 - protein, 73 aa, synthetic construct
PEPRVSTEHTNNKIEKIYIMKADTVIVGTVKAELPEGRPEQETEPPLGSCSDVMLSVEEEGKEDPLPTAASGK

SEQ ID NO: 41 - DNA, 1593 bp, synthetic construct
  source 1..1593: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggctcgacgagcggctctggtaaaccgggctctggtgaaggcagtaccaaaggtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcctgagccccgggtgtccacggagcacaccaataacaagattgagaaaatctacatcatgaaggctgacaccgtgatcgtggggaccgtgaaggctgagctgccggagggccggcccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 42 - protein, 530 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGSTSGSGKPGSGEGSTKGEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAATTTPAPRPPTPAPTIASQPLSLRPEACRPAAGGAVHTRGLDFACDFWVLVVVGGVLACYSLLVTVAFIIFWVRSKPEPRVSTEHTNNKIEKIYIMKADTVIVGTVKAELPEGRPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 171 bp, synthetic construct
  source 1..171: mol_type = other DNA
ggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 44 - protein, 57 aa, synthetic construct
GLAGPAEPELEEELEADHTPHYPEQETEPPLGSCSDVMLSVEEEGKEDPLPTAASGK

SEQ ID NO: 45 - DNA, 135 bp, synthetic construct
  source 1..135: mol_type = other DNA
gcagagcccgagttggaggaggagctggaggcggaccataccccccactaccccgagcaggagacagaaccgcctctgggcagctgcagcgatgtcatgctctcagtggaagaggaagggaaagaagaccccttg

SEQ ID NO: 46 - protein, 45 aa, synthetic construct
AEPELEEELEADHTPHYPEQETEPPLGSCSDVMLSVEEEGKEDPL

SEQ ID NO: 47 - DNA, 183 bp, synthetic construct
  source 1..183: mol_type = other DNA
gcagagcccgagttggaggaggagctggaggcggaccataccccccactaccccgagcaggagacagaaccgcctctgggcagctgcagcgatgtcatgctctcagtggaagaggaagggaaagaagaccccttgcacagtgactacatgaacatgactccccgccgccccgggcccacccgc

SEQ ID NO: 48 - protein, 61 aa, synthetic construct
AEPELEEELEADHTPHYPEQETEPPLGSCSDVMLSVEEEGKEDPLHSDYMNMTPRRPGPTR

SEQ ID NO: 49 - DNA, 183 bp, synthetic construct
  source 1..183: mol_type = other DNA
gcagagcccgagttggaggaggagctggaggcggaccataccccccactaccccgagcaggagacagaaccgcctctgggcagctgcagcgatgtcatgctctcagtggaagaggaagggaaagaagaccccttgcacagtgactacatgttcatgactccccgccgccccgggcccacccgc

SEQ ID NO: 50 - protein, 61 aa, synthetic construct
AEPELEEELEADHTPHYPEQETEPPLGSCSDVMLSVEEEGKEDPLHSDYMFMTPRRPGPTR

SEQ ID NO: 51 - DNA, 1545 bp, synthetic construct
  source 1..1545: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggctcgacgagcggctctggtaaaccgggctctggtgaaggcagtaccaaaggtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 52 - protein, 514 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGSTSGSGKPGSGEGSTKGEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAATTTPAPRPPTPAPTIASQPLSLRPEACRPAAGGAVHTRGLDFACDFWVLVVVGGVLACYSLLVTVAFIIFWVRSKGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 45 bp, synthetic construct
  source 1..45: mol_type = other DNA
ggcggcggcggcagcggaggaggaggaagcggaggaggcggcagc

SEQ ID NO: 54 - protein, 15 aa, synthetic construct
GGGGSGGGGSGGGGS

SEQ ID NO: 55 - DNA, 1518 bp, synthetic construct
  source 1..1518: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 56 - protein, 505 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - DNA, 1482 bp, synthetic construct
  source 1..1482: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaaggcagagcccgagttggaggaggagctggaggcggaccataccccccactaccccgagcaggagacagaaccgcctctgggcagctgcagcgatgtcatgctctcagtggaagaggaagggaaagaagaccccttgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 58 - protein, 493 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKAEPELEEELEADHTPHYPEQETEPPLGSCSDVMLSVEEEGKEDPLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - DNA, 1530 bp, synthetic construct
  source 1..1530: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaaggcagagcccgagttggaggaggagctggaggcggaccataccccccactaccccgagcaggagacagaaccgcctctgggcagctgcagcgatgtcatgctctcagtggaagaggaagggaaagaagaccccttgcacagtgactacatgaacatgactccccgccgccccgggcccacccgccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 60 - protein, 509 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKAEPELEEELEADHTPHYPEQETEPPLGSCSDVMLSVEEEGKEDPLHSDYMNMTPRRPGPTRRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 1530 bp, synthetic construct
  source 1..1530: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaaggcagagcccgagttggaggaggagctggaggcggaccataccccccactaccccgagcaggagacagaaccgcctctgggcagctgcagcgatgtcatgctctcagtggaagaggaagggaaagaagaccccttgcacagtgactacatgttcatgactccccgccgccccgggcccacccgccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 62 - protein, 509 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKAEPELEEELEADHTPHYPEQETEPPLGSCSDVMLSVEEEGKEDPLHSDYMFMTPRRPGPTRRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - DNA, 1461 bp, synthetic construct
  source 1..1461: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 64 - protein, 486 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - DNA, 126 bp, synthetic construct
  source 1..126: mol_type = other DNA
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 66 - protein, 42 aa, synthetic construct
KRGRKKLLYIFKQPFMRPVQTTQEEDGCSCRFPEEEEGGCEL

SEQ ID NO: 67 - DNA, 1464 bp, synthetic construct
  source 1..1464: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 68 - protein, 487 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 141 bp, synthetic construct
  source 1..141: mol_type = other DNA
cgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 70 - protein, 47 aa, synthetic construct
RRKYRSNKGESPVEPAEPCRYSCPREEEGSTIPIQEDYRKPEPACSP

SEQ ID NO: 71 - DNA, 1479 bp, synthetic construct
  source 1..1479: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgcgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 72 - protein, 492 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRRKYRSNKGESPVEPAEPCRYSCPREEEGSTIPIQEDYRKPEPACSPRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - DNA, 114 bp, synthetic construct
  source 1..114: mol_type = other DNA
tgttggcttacaaaaaagaagtattcatccagtgtgcacgaccctaacggtgaatacatgttcatgagagcagtgaacacagccaaaaaatctagactcacagatgtgacccta

SEQ ID NO: 74 - protein, 38 aa, synthetic construct
CWLTKKKYSSSVHDPNGEYMFMRAVNTAKKSRLTDVTL

SEQ ID NO: 75 - DNA, 1452 bp, synthetic construct
  source 1..1452: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgtgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 76 - protein, 483 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - DNA, 126 bp, synthetic construct
  source 1..126: mol_type = other DNA
gccctgtacctgctccggagggaccagaggctgccccccgatgcccacaagccccctgggggaggcagtttccggacccccatccaagaggagcaggccgacgcccactccaccctggccaagatc

SEQ ID NO: 78 - protein, 42 aa, synthetic construct
ALYLLRRDQRLPPDAHKPPGGGSFRTPIQEEQADAHSTLAKI

SEQ ID NO: 79 - DNA, 1464 bp, synthetic construct
  source 1..1464: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtggccctgtacctgctccggagggaccagaggctgccccccgatgcccacaagccccctgggggaggcagtttccggacccccatccaagaggagcaggccgacgcccactccaccctggccaagatccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 80 - protein, 487 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - DNA, 1587 bp, synthetic construct
  source 1..1587: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 82 - protein, 528 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 1644 bp, synthetic construct
  source 1..1644: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 84 - protein, 547 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - DNA, 1632 bp, synthetic construct
  source 1..1632: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 86 - protein, 543 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 1632 bp, synthetic construct
  source 1..1632: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagtgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 88 - protein, 543 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - DNA, 1659 bp, synthetic construct
  source 1..1659: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 90 - protein, 552 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKRRKYRSNKGESPVEPAEPCRYSCPREEEGSTIPIQEDYRKPEPACSPRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 1644 bp, synthetic construct
  source 1..1644: mol_type = other DNA
atgcttctcctggtgacaagccttctgctctgtgagttaccacacccagcattcctcctgatcccagatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggcggcggcggcagcggaggaggaggaagcggaggaggcggcagcgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaaggccctgtacctgctccggagggaccagaggctgccccccgatgcccacaagccccctgggggaggcagtttccggacccccatccaagaggagcaggccgacgcccactccaccctggccaagatccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 92 - protein, 547 aa, synthetic construct
MLLLVTSLLLCELPHPAFLLIPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAAIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - DNA, 63 bp, synthetic construct
  source 1..63: mol_type = other DNA
atggccttaccagtgaccgccttgctcctgccgctggccttgctgctccacgccgccaggccg

SEQ ID NO: 94 - protein, 21 aa, synthetic construct
MALPVTALLLPLALLLHAARP

SEQ ID NO: 95 - DNA, 336 bp, synthetic construct
  source 1..336: mol_type = other DNA
gacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaag

SEQ ID NO: 96 - protein, 112 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 97 - DNA, 348 bp, synthetic construct
  source 1..348: mol_type = other DNA
gaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctcc

SEQ ID NO: 98 - protein, 116 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 99 - DNA, 1452 bp, synthetic construct
  source 1..1452: mol_type = other DNA
atggccttaccagtgaccgccttgctcctgccgctggccttgctgctccacgccgccaggccggacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaagggcggcggcggcagcggaggaggaggaagcggaggaggcggcagcgaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctccattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 100 - protein, 483 aa, synthetic construct
MALPVTALLLPLALLLHAARPDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKGGGGSGGGGSGGGGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - DNA, 1464 bp, synthetic construct
  source 1..1464: mol_type = other DNA
atggccttaccagtgaccgccttgctcctgccgctggccttgctgctccacgccgccaggccggacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaagggcggcggcggcagcggaggaggaggaagcggaggaggcggcagcgaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctccattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 102 - protein, 487 aa, synthetic construct
MALPVTALLLPLALLLHAARPDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKGGGGSGGGGSGGGGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSIEVMYPPPYLDNEKSNGTIIHVKGKHLCPSPLFPGPSKPFWVLVVVGGVLACYSLLVTVAFIIFWVRSKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - DNA, 1509 bp, synthetic construct
  source 1..1509: mol_type = other DNA
atggccttaccagtgaccgccttgctcctgccgctggccttgctgctccacgccgccaggccggacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaagggcggcggcggcagcggaggaggaggaagcggaggaggcggcagcgaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctccattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 104 - protein, 502 aa, synthetic construct
MALPVTALLLPLALLLHAARPDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKGGGGSGGGGSGGGGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - DNA, 1008 bp, synthetic construct
  source 1..1008: mol_type = other DNA
atggccttaccagtgaccgccttgctcctgccgctggccttgctgctccacgccgccaggccggacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaagggcggcggcggcagcggaggaggaggaagcggaggaggcggcagcgaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctccattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaaggcggcctga

SEQ ID NO: 106 - protein, 335 aa, synthetic construct
MALPVTALLLPLALLLHAARPDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKGGGGSGGGGSGGGGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSIEVMYPPPYLDNEKSNGTIIHVKGKHLCPSPLFPGPSKPFWVLVVVGGVLACYSLLVTVAFIIFWVRSKAA

SEQ ID NO: 107 - DNA, 1623 bp, synthetic construct
  source 1..1623: mol_type = other DNA
atggccttaccagtgaccgccttgctcctgccgctggccttgctgctccacgccgccaggccggacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaagggcggcggcggcagcggaggaggaggaagcggaggaggcggcagcgaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctccattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 108 - protein, 540 aa, synthetic construct
MALPVTALLLPLALLLHAARPDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKGGGGSGGGGSGGGGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - DNA, 1578 bp, synthetic construct
  source 1..1578: mol_type = other DNA
atggccttaccagtgaccgccttgctcctgccgctggccttgctgctccacgccgccaggccggacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaagggcggcggcggcagcggaggaggaggaagcggaggaggcggcagcgaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctccattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 110 - protein, 525 aa, synthetic construct
MALPVTALLLPLALLLHAARPDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKGGGGSGGGGSGGGGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - DNA, 1635 bp, synthetic construct
  source 1..1635: mol_type = other DNA
atggccttaccagtgaccgccttgctcctgccgctggccttgctgctccacgccgccaggccggacatccagatgacccagtcccccagcagcctgtccgccagcgtgggagacagggtgacaatcacctgcaggagcacaaagagcctgctgcactccaacggcatcacctacctgtactggtaccagcagaagcctggcaaggcccccaagctgctgatctaccagatgagcaatctggccagcggcgtgccttccaggttttccagctccggcagcggcaccgattttaccctgacaatctcctccctgcagcctgaggatttcgccacatactactgcgcccagaatctggagatccctagaaccttcggccagggcacaaaggtggagctgaagggcggcggcggcagcggaggaggaggaagcggaggaggcggcagcgaggtgcagctggtgcagtccggccctggcctggtgcagcctggaggaagcgtgagaatcagctgcgccgcctccggctacacctttaccaattacggcatgaattgggtgaagcaggcccctggcaagggcctggagtggatgggctggatcaatacctacaccggcgagtccacatacgccgactcctttaagggcagattcaccttcagcctggatacaagcgcctccgccgcctacctgcagatcaactccctgagagccgaggacaccgccgtgtactactgtgccagatttgccatcaagggcgactactggggccagggcaccctgctgaccgtgtcctccattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 112 - protein, 544 aa, synthetic construct
MALPVTALLLPLALLLHAARPDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKGGGGSGGGGSGGGGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 60 bp, synthetic construct
  source 1..60: mol_type = other DNA
atgcagatcccacaggcgccctggccagtcgtctgggcggtgctacaactgggctggcgg

SEQ ID NO: 114 - protein, 20 aa, synthetic construct
MQIPQAPWPVVWAVLQLGWR

SEQ ID NO: 115 - DNA, 450 bp, synthetic construct
  source 1..450: mol_type = other DNA
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg

SEQ ID NO: 116 - protein, 150 aa, synthetic construct [sequence omitted]

SEQ ID NO: 117 - DNA, 15 bp, synthetic construct
  source 1..15: mol_type = other DNA
gttggtgtcgtgggc

SEQ ID NO: 118 - protein, 5 aa, synthetic construct
VGVVG

SEQ ID NO: 119 - DNA, 1068 bp, synthetic construct
  source 1..1068: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 120 - protein, 355 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - DNA, 1071 bp, synthetic construct
  source 1..1071: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcttttgggtgctggtggtggttgggggagtcctggcttgctatagcttgctagtaacagtggcctttattattttctgggtgaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 122 - protein, 356 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1125 bp, synthetic construct
  source 1..1125: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 124 - protein, 374 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGFWVLVVVGGVLACYSLLVTVAFIIFWVRSKGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - DNA, 624 bp, synthetic construct
  source 1..624: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcttttgggtgctggtggtggttgggggagtcctggcttgctatagcttgctagtaacagtggcctttattattttctgggtgaggagcaaggcggcctga

SEQ ID NO: 126 - protein, 207 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGFWVLVVVGGVLACYSLLVTVAFIIFWVRSKAA

SEQ ID NO: 127 - DNA, 1239 bp, synthetic construct
  source 1..1239: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 128 - protein, 412 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGFWVLVVVGGVLACYSLLVTVAFIIFWVRSKRSRLLHSDYMNMTPRRPGPTRKHYQPYAPPRDFAAYRSGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1194 bp, synthetic construct
  source 1..1194: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 130 - protein, 397 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - DNA, 1242 bp, synthetic construct
  source 1..1242: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcttttgggtgctggtggtggttgggggagtcctggcttgctatagcttgctagtaacagtggcctttattattttctgggtgggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 132 - protein, 413 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGFWVLVVVGGVLACYSLLVTVAFIIFWVGLAGPAEPELEEELEADHTPHYPEQETEPPLGSCSDVMLSVEEEGKEDPLPTAASGKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - DNA, 357 bp, synthetic construct
  source 1..357: mol_type = other DNA
caggtgcagctgcagcagagcggccctgagctggagaagcctggcgccagcgtgaagattagctgtaaggccagcggctatagctttacaggatatacaatgaattgggtgaagcagagccatggcaagtctctggagtggattggcctgattacaccttataatggcgccagctcttataatcagaagtttagaggcaaggccacactgacagtggataagtctagctctacagcctatatggatctgctgagcctgacatctgaggattctgccgtgtatttttgtgccagaggcggctatgatggcagaggctttgattattggggctctggcacacctgtgacagtgtctagc

SEQ ID NO: 134 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 135 - DNA, 318 bp, synthetic construct
  source 1..318: mol_type = other DNA
gatattgagctgacacagtctcctgccattatgagcgcctctccaggcgagaaggtgacaatgacatgttctgccagctctagcgtgagctatatgcactggtatcagcagaagtctggcacaagccctaagagatggatctatgatacctctaagctggcctctggagtgcctggcagattttctggcagcggatctggaaatagctattctctgaccatctcttctgtggaggccgaggatgatgccacatattattgtcagcagtggtctaagcaccctctgacatttggatctggcaccaaagtggagatcaag

SEQ ID NO: 136 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 137 - DNA, 1446 bp, synthetic construct
  source 1..1446: mol_type = other DNA
atgcttctcctggtgacaagccttctgctctgtgagttaccacacccagcattcctcctgatcccacaggtgcagctgcagcagagcggccctgagctggagaagcctggcgccagcgtgaagattagctgtaaggccagcggctatagctttacaggatatacaatgaattgggtgaagcagagccatggcaagtctctggagtggattggcctgattacaccttataatggcgccagctcttataatcagaagtttagaggcaaggccacactgacagtggataagtctagctctacagcctatatggatctgctgagcctgacatctgaggattctgccgtgtatttttgtgccagaggcggctatgatggcagaggctttgattattggggctctggcacacctgtgacagtgtctagcggcggcggcggcagcggaggaggaggaagcggaggaggcggcagcgatattgagctgacacagtctcctgccattatgagcgcctctccaggcgagaaggtgacaatgacatgttctgccagctctagcgtgagctatatgcactggtatcagcagaagtctggcacaagccctaagagatggatctatgatacctctaagctggcctctggagtgcctggcagattttctggcagcggatctggaaatagctattctctgaccatctcttctgtggaggccgaggatgatgccacatattattgtcagcagtggtctaagcaccctctgacatttggatctggcaccaaagtggagatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 138 - protein, 481 aa, synthetic construct
MLLLVTSLLLCELPHPAFLLIPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - DNA, 1449 bp, synthetic construct
  source 1..1449: mol_type = other DNA
atgcttctcctggtgacaagccttctgctctgtgagttaccacacccagcattcctcctgatcccacaggtgcagctgcagcagagcggccctgagctggagaagcctggcgccagcgtgaagattagctgtaaggccagcggctatagctttacaggatatacaatgaattgggtgaagcagagccatggcaagtctctggagtggattggcctgattacaccttataatggcgccagctcttataatcagaagtttagaggcaaggccacactgacagtggataagtctagctctacagcctatatggatctgctgagcctgacatctgaggattctgccgtgtatttttgtgccagaggcggctatgatggcagaggctttgattattggggctctggcacacctgtgacagtgtctagcggcggcggcggcagcggaggaggaggaagcggaggaggcggcagcgatattgagctgacacagtctcctgccattatgagcgcctctccaggcgagaaggtgacaatgacatgttctgccagctctagcgtgagctatatgcactggtatcagcagaagtctggcacaagccctaagagatggatctatgatacctctaagctggcctctggagtgcctggcagattttctggcagcggatctggaaatagctattctctgaccatctcttctgtggaggccgaggatgatgccacatattattgtcagcagtggtctaagcaccctctgacatttggatctggcaccaaagtggagatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 140 - protein, 482 aa, synthetic construct
MLLLVTSLLLCELPHPAFLLIPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - DNA, 1503 bp, synthetic construct
  source 1..1503: mol_type = other DNA
atgcttctcctggtgacaagccttctgctctgtgagttaccacacccagcattcctcctgatcccacaggtgcagctgcagcagagcggccctgagctggagaagcctggcgccagcgtgaagattagctgtaaggccagcggctatagctttacaggatatacaatgaattgggtgaagcagagccatggcaagtctctggagtggattggcctgattacaccttataatggcgccagctcttataatcagaagtttagaggcaaggccacactgacagtggataagtctagctctacagcctatatggatctgctgagcctgacatctgaggattctgccgtgtatttttgtgccagaggcggctatgatggcagaggctttgattattggggctctggcacacctgtgacagtgtctagcggcggcggcggcagcggaggaggaggaagcggaggaggcggcagcgatattgagctgacacagtctcctgccattatgagcgcctctccaggcgagaaggtgacaatgacatgttctgccagctctagcgtgagctatatgcactggtatcagcagaagtctggcacaagccctaagagatggatctatgatacctctaagctggcctctggagtgcctggcagattttctggcagcggatctggaaatagctattctctgaccatctcttctgtggaggccgaggatgatgccacatattattgtcagcagtggtctaagcaccctctgacatttggatctggcaccaaagtggagatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 142 - protein, 500 aa, synthetic construct
MLLLVTSLLLCELPHPAFLLIPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - DNA, 1617 bp, synthetic construct
  source 1..1617: mol_type = other DNA
atgcttctcctggtgacaagccttctgctctgtgagttaccacacccagcattcctcctgatcccacaggtgcagctgcagcagagcggccctgagctggagaagcctggcgccagcgtgaagattagctgtaaggccagcggctatagctttacaggatatacaatgaattgggtgaagcagagccatggcaagtctctggagtggattggcctgattacaccttataatggcgccagctcttataatcagaagtttagaggcaaggccacactgacagtggataagtctagctctacagcctatatggatctgctgagcctgacatctgaggattctgccgtgtatttttgtgccagaggcggctatgatggcagaggctttgattattggggctctggcacacctgtgacagtgtctagcggcggcggcggcagcggaggaggaggaagcggaggaggcggcagcgatattgagctgacacagtctcctgccattatgagcgcctctccaggcgagaaggtgacaatgacatgttctgccagctctagcgtgagctatatgcactggtatcagcagaagtctggcacaagccctaagagatggatctatgatacctctaagctggcctctggagtgcctggcagattttctggcagcggatctggaaatagctattctctgaccatctcttctgtggaggccgaggatgatgccacatattattgtcagcagtggtctaagcaccctctgacatttggatctggcaccaaagtggagatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 144 - protein, 538 aa, synthetic construct
MLLLVTSLLLCELPHPAFLLIPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - DNA, 1572 bp, synthetic construct
  source 1..1572: mol_type = other DNA
atgcttctcctggtgacaagccttctgctctgtgagttaccacacccagcattcctcctgatcccacaggtgcagctgcagcagagcggccctgagctggagaagcctggcgccagcgtgaagattagctgtaaggccagcggctatagctttacaggatatacaatgaattgggtgaagcagagccatggcaagtctctggagtggattggcctgattacaccttataatggcgccagctcttataatcagaagtttagaggcaaggccacactgacagtggataagtctagctctacagcctatatggatctgctgagcctgacatctgaggattctgccgtgtatttttgtgccagaggcggctatgatggcagaggctttgattattggggctctggcacacctgtgacagtgtctagcggcggcggcggcagcggaggaggaggaagcggaggaggcggcagcgatattgagctgacacagtctcctgccattatgagcgcctctccaggcgagaaggtgacaatgacatgttctgccagctctagcgtgagctatatgcactggtatcagcagaagtctggcacaagccctaagagatggatctatgatacctctaagctggcctctggagtgcctggcagattttctggcagcggatctggaaatagctattctctgaccatctcttctgtggaggccgaggatgatgccacatattattgtcagcagtggtctaagcaccctctgacatttggatctggcaccaaagtggagatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 146 - protein, 523 aa, synthetic construct
MLLLVTSLLLCELPHPAFLLIPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - DNA, 1629 bp, synthetic construct
  source 1..1629: mol_type = other DNA
atgcttctcctggtgacaagccttctgctctgtgagttaccacacccagcattcctcctgatcccacaggtgcagctgcagcagagcggccctgagctggagaagcctggcgccagcgtgaagattagctgtaaggccagcggctatagctttacaggatatacaatgaattgggtgaagcagagccatggcaagtctctggagtggattggcctgattacaccttataatggcgccagctcttataatcagaagtttagaggcaaggccacactgacagtggataagtctagctctacagcctatatggatctgctgagcctgacatctgaggattctgccgtgtatttttgtgccagaggcggctatgatggcagaggctttgattattggggctctggcacacctgtgacagtgtctagcggcggcggcggcagcggaggaggaggaagcggaggaggcggcagcgatattgagctgacacagtctcctgccattatgagcgcctctccaggcgagaaggtgacaatgacatgttctgccagctctagcgtgagctatatgcactggtatcagcagaagtctggcacaagccctaagagatggatctatgatacctctaagctggcctctggagtgcctggcagattttctggcagcggatctggaaatagctattctctgaccatctcttctgtggaggccgaggatgatgccacatattattgtcagcagtggtctaagcaccctctgacatttggatctggcaccaaagtggagatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 148 - protein, 542 aa, synthetic construct
MLLLVTSLLLCELPHPAFLLIPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - DNA, 54 bp, synthetic construct
  source 1..54: mol_type = other DNA
atggcctggatgctgttgctcatcttgatcatggtccatccaggatcctgtgct

SEQ ID NO: 150 - protein, 18 aa, synthetic construct
MAWMLLLILIMVHPGSCA

SEQ ID NO: 151 - DNA, 351 bp, synthetic construct
  source 1..351: mol_type = other DNA
ctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 152 - protein, 117 aa, synthetic construct
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLG

SEQ ID NO: 153 - DNA, 15 bp, synthetic construct
  source 1..15: mol_type = other DNA
gctggtacagtcctc

SEQ ID NO: 154 - protein, 5 aa, synthetic construct
AGTVL

SEQ ID NO: 155 - DNA, 1134 bp, synthetic construct
  source 1..1134: mol_type = other DNA
atggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 156 - protein, 377 aa, synthetic construct
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLFWVLVVVGGVLACYSLLVTVAFIIFWVRSKRSRLLHSDYMNMTPRRPGPTRKHYQPYAPPRDFAAYRSGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - DNA, 1089 bp, synthetic construct
  source 1..1089: mol_type = other DNA
atggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 158 - protein, 362 aa, synthetic construct
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - DNA, 1146 bp, synthetic construct
  source 1..1146: mol_type = other DNA
atggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 160 - protein, 381 aa, synthetic construct
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLFWVLVVVGGVLACYSLLVTVAFIIFWVRSKGLAGPAEPELEEELEADHTPHYPEQETEPPLGSCSDVMLSVEEEGKEDPLPTAASGKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - DNA, 57 bp, synthetic construct
  source 1..57: mol_type = other DNA
atggattggatttggcgcattctgtttctggtgggagccgcaaccggagcacatagt

SEQ ID NO: 162 - protein, 19 aa, synthetic construct
MDWIWRILFLVGAATGAHS

SEQ ID NO: 163 - DNA, 345 bp, synthetic construct
  source 1..345: mol_type = other DNA
caggtccagctgcagcagtcaggagccgaactggtgcggcccggcgcaagtgtcaaactgtcatgcaaggccagcgggtataccttcacagactacgagatgcactgggtgaaacagacccctgtgcacggcctgaagtggatcggcgctctggacccaaaaaccggggatacagcatattcccagaagtttaaaggaaaggccactctgaccgctgacaagagctcctctactgcctacatggagctgaggagcctgacatccgaagatagcgccgtgtactattgcacccgcttctactcctatacatactggggccaggggactctggtgaccgtctctgca

SEQ ID NO: 164 - protein, 115 aa, synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 165 - DNA, 336 bp, synthetic construct
  source 1..336: mol_type = other DNA
gacgtggtcatgacacagactccactgtccctgcccgtgagcctgggcgatcaggctagcatttcctgtcgaagttcacagagtctggtgcactcaaacggaaatacctatctgcattggtacctgcagaagccaggccagtctcccaaactgctgatctataaggtgagcaaccggttctccggggtccctgacagattttctggaagtggctcagggacagatttcactctgaaaattagcagagtggaggccgaagatctgggcgtctacttttgtagccagaatacccacgtcccaccaacattcggaagcggcactaaactggaaatcaag

SEQ ID NO: 166 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 167 - DNA, 1614 bp, synthetic construct
  source 1..1614: mol_type = other DNA
atggattggatttggcgcattctgtttctggtgggagccgcaaccggagcacatagtcaggtccagctgcagcagtcaggagccgaactggtgcggcccggcgcaagtgtcaaactgtcatgcaaggccagcgggtataccttcacagactacgagatgcactgggtgaaacagacccctgtgcacggcctgaagtggatcggcgctctggacccaaaaaccggggatacagcatattcccagaagtttaaaggaaaggccactctgaccgctgacaagagctcctctactgcctacatggagctgaggagcctgacatccgaagatagcgccgtgtactattgcacccgcttctactcctatacatactggggccaggggactctggtgaccgtctctgcaggaggaggaggctctggaggaggagggagtggaggcgggggaagcgacgtggtcatgacacagactccactgtccctgcccgtgagcctgggcgatcaggctagcatttcctgtcgaagttcacagagtctggtgcactcaaacggaaatacctatctgcattggtacctgcagaagccaggccagtctcccaaactgctgatctataaggtgagcaaccggttctccggggtccctgacagattttctggaagtggctcagggacagatttcactctgaaaattagcagagtggaggccgaagatctgggcgtctacttttgtagccagaatacccacgtcccaccaacattcggaagcggcactaaactggaaatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 168 - protein, 537 aa, synthetic construct
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - DNA, 1569 bp, synthetic construct
  source 1..1569: mol_type = other DNA
atggattggatttggcgcattctgtttctggtgggagccgcaaccggagcacatagtcaggtccagctgcagcagtcaggagccgaactggtgcggcccggcgcaagtgtcaaactgtcatgcaaggccagcgggtataccttcacagactacgagatgcactgggtgaaacagacccctgtgcacggcctgaagtggatcggcgctctggacccaaaaaccggggatacagcatattcccagaagtttaaaggaaaggccactctgaccgctgacaagagctcctctactgcctacatggagctgaggagcctgacatccgaagatagcgccgtgtactattgcacccgcttctactcctatacatactggggccaggggactctggtgaccgtctctgcaggaggaggaggctctggaggaggagggagtggaggcgggggaagcgacgtggtcatgacacagactccactgtccctgcccgtgagcctgggcgatcaggctagcatttcctgtcgaagttcacagagtctggtgcactcaaacggaaatacctatctgcattggtacctgcagaagccaggccagtctcccaaactgctgatctataaggtgagcaaccggttctccggggtccctgacagattttctggaagtggctcagggacagatttcactctgaaaattagcagagtggaggccgaagatctgggcgtctacttttgtagccagaatacccacgtcccaccaacattcggaagcggcactaaactggaaatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 170 - protein, 522 aa, synthetic construct
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 1626 bp, synthetic construct
  source 1..1626: mol_type = other DNA
atggattggatttggcgcattctgtttctggtgggagccgcaaccggagcacatagtcaggtccagctgcagcagtcaggagccgaactggtgcggcccggcgcaagtgtcaaactgtcatgcaaggccagcgggtataccttcacagactacgagatgcactgggtgaaacagacccctgtgcacggcctgaagtggatcggcgctctggacccaaaaaccggggatacagcatattcccagaagtttaaaggaaaggccactctgaccgctgacaagagctcctctactgcctacatggagctgaggagcctgacatccgaagatagcgccgtgtactattgcacccgcttctactcctatacatactggggccaggggactctggtgaccgtctctgcaggaggaggaggctctggaggaggagggagtggaggcgggggaagcgacgtggtcatgacacagactccactgtccctgcccgtgagcctgggcgatcaggctagcatttcctgtcgaagttcacagagtctggtgcactcaaacggaaatacctatctgcattggtacctgcagaagccaggccagtctcccaaactgctgatctataaggtgagcaaccggttctccggggtccctgacagattttctggaagtggctcagggacagatttcactctgaaaattagcagagtggaggccgaagatctgggcgtctacttttgtagccagaatacccacgtcccaccaacattcggaagcggcactaaactggaaatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 172 - protein, 541 aa, synthetic construct
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - DNA, 332 bp, synthetic construct
  source 1..332: mol_type = other DNA
gacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaa

SEQ ID NO: 174 - protein, 110 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEI

SEQ ID NO: 175 - DNA, 366 bp, synthetic construct
  source 1..366: mol_type = other DNA
aaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgca

SEQ ID NO: 176 - protein, 122 aa, synthetic construct
KGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAA

SEQ ID NO: 177 - DNA, 1650 bp, synthetic construct
  source 1..1650: mol_type = other DNA
atgcttctcctggtgacaagccttctgctctgtgagttaccacacccagcattcctcctgatcccagacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggcggcggcggcagcggaggaggaggaagcggaggaggcggcagcaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 178 - protein, 549 aa, synthetic construct
MLLLVTSLLLCELPHPAFLLIPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSGGGGSGGGGSKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSGLAGPAEPELEEELEADHTPHYPEQETEPPLGSCSDVMLSVEEEGKEDPLPTAASGKRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 179 - DNA, 1605 bp, synthetic construct
  source 1..1605: mol_type = other DNA
atgcttctcctggtgacaagccttctgctctgtgagttaccacacccagcattcctcctgatcccagacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggcggcggcggcagcggaggaggaggaagcggaggaggcggcagcaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 180 - protein, 534 aa, synthetic construct
MLLLVTSLLLCELPHPAFLLIPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSGGGGSGGGGSKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - DNA, 1662 bp, synthetic construct
  source 1..1662: mol_type = other DNA
atgcttctcctggtgacaagccttctgctctgtgagttaccacacccagcattcctcctgatcccagacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggcggcggcggcagcggaggaggaggaagcggaggaggcggcagcaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagcaagggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 182 - protein, 553 aa, synthetic construct
MLLLVTSLLLCELPHPAFLLIPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSGGGGSGGGGSKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAAAAIEVMYPPPYLDNEKSNGTIIHVKGKHLCPSPLFPGPSKPFWVLVVVGGVLACYSLLVTVAFIIFWVRSKGLAGPAEPELEEELEADHTPHYPEQETEPPLGSCSDVMLSVEEEGKEDPLPTAASGKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR